(19) United States
(12) Patent Application Publication
(10) Pub. No.: US 2023/0090708 A1
(43) Pub. Date: Mar. 23, 2023

(54) METHODS AND COMPOSITIONS FOR THE TREATMENT OF HEMANGIOMA

(71) Applicants: MASSEY VENTURES LIMITED, Palmerston North (NZ); GILLIES MCINDOE RESEARCH INSTITUTE, Wellington (NZ)

(72) Inventors: Paul Frank DAVIS, Wellington (NZ); Sean Marshall MACKAY, Palmerston North (NZ); Erin Fay PATERSON, Paraparaumu (NZ); Swee Thong TAN, Wellington (NZ); Eng Wui TAN, Dunedin (NZ)

(21) Appl. No.: 17/795,779

(22) PCT Filed: Jan. 29, 2021
(86) PCT No.: PCT/NZ2021/050012

§ 371 Date: Jul. 27, 2022

(30) Foreign Application Priority Data
Jan. 29, 2020 (NZ) 761251

(51) Int. Cl. A61K 31/401; A61K 31/138; A61K 9/00

(52) U.S. Cl. CPC A61K 31/401; A61K 31/138; A61K 9/0014

(57) ABSTRACT

The present invention relates to methods and compositions for the treatment of hemangioma, and particularly, but not exclusively, methods and compositions for the treatment of infantile hemangioma. In certain aspects, the methods comprise locally administering an ACE inhibitor or an ATIIR2 antagonist to a subject. In other aspects, the methods comprise systemically administering two or more of an ACE inhibitor, a beta-blocker and an ATIIR2 antagonist. The present invention also relates to compositions that are suitable for local administration and comprise: an ACEi and a beta-blocker; an ACEi and an ATIIR2 antagonist; a beta-blocker and an AT11R2 antagonist; or, an ACEi, a beta-blocker, and an AT11R2 antagonist.

### FIELD

The present invention relates to methods and compositions for the treatment of hemangioma, and particularly, but not exclusively, methods and compositions for the treatment of infantile hemangioma.

### BACKGROUND

Haemangiomas are complex growths which develop as the result of proliferation of endothelial cells surrounding blood-filled cavities. They can occur anywhere on the body, but most commonly appear on the face, scalp, chest or back. Hemangiomas will generally slowly fade over time and most do not require treatment. However, they can be cosmetically disfiguring, psychologically disturbing, and can cause functional disability dependant on the location of the lesion; for example, blindness if it occurs in the tissue surrounding the eye. In these cases, treatment is necessary to avoid permanent morbidity.

Infantile haemangioma (IH), affecting up to 10% of children, is the most common type of hemangioma. IH is characterised by an initial rapid proliferation phase, followed by a slow spontaneous involution over 5 to 10 years, which often leads a fibro-fatty residuum (Itinteang et al., Plast. Reconstr. Surg. 2011; 128, 499-507).

The current standard of care for treating proliferating IH involves orally administering a low dose β-blocker, such as propranolol. This approach has been relatively successful, with approximately 88% of patients treated showing an improvement within 5 weeks and overall approximately 60% of patients are successfully treated (Léauté-Labrèze et al. N. Engl. J. Med., 2015; 372, 735-746). However, due to the potential intolerable side effects which can occur during β-blocker therapy (Ji et al. Sci. Rep. 2018; 8, 4264), management of IH is largely conservative, generally treating only the 10-15% of patients who require intervention during infancy due to the threat of the lesion to life or function, or tissue distortion or destruction leading to physical disfiguration. Furthermore, rebound or regrowth of the haemangioma lesion, defined as an increase in size, change in colour, or both, can occur after discontinuation of oral propranolol treatment, particularly when treatment is discontinued before the age of 1 year (Price et al., 2011, Arch. Dermatol., 147, 1371-1376; Menezes et al., 2011, Ann. Otol. Rhinol. Laryngol., 120, 686-695).

A number of clinical studies have been performed to investigate the effect of orally administered captopril, an ACE (angiotensin converting enzyme) inhibitor (ACEi), on proliferating IH. In one study in eight patients, captopril was administered at a dosage of 1.5 mg/kg daily, demonstrating a positive effect in all patients (Tan et al. Br. J. Dermatol. 2012; 167, 619-624). The response varied between patients, and scored as dramatic (n=3), medium (n=2), and slow (n=3), and treatment was ceased after 14 months, after which no rebound growth was observed. However, a randomised controlled trial has been performed comparing the efficacy of orally administered propranolol versus captopril in 30 patients with IH and 35 healthy control subjects, demonstrating that clinical improvement is significantly better and faster in patients treated with propranolol (Zaher et al. 2016, J. Am. Acad. Dermatol., 74, 499-505).

Local treatment options to control the growth of proliferating IH have been investigated but are currently unavailable for clinical use and have a number of unwanted side-effects. Topically applied ultrapotent corticosteroids, for example 0.05% clobetasol propionate, has been used to treat flat or minimally raised IH lesions in 27 infants for a period of 4 to 21 weeks with varied clinical improvements observed (35% good response, 38% partial response, 27% no response) (Garzon et al. 2005, J. Am. Acad. Dermatol., 52, 281-286). However, adverse effects can include localised atrophy, hypopigmentation, hypertrichosis and infection. Imiquimod, an immune response modifier with anti-angiogenic and pro-apoptotic properties, has been used as a topical treatment for superficial IH. Generally, all cases of superficial IH are improved within 4 months of treatment with 5% imiquimod cream (Ho et al. 2007, J. Am. Acad. Dermatol., 56, 63-68). However, side effects can include severe local inflammatory reactions leading to disfiguring scars (Qiu et al. 2013, Pediatr. Dermatol., 30, 342-347).

Topically applied β-blockers have also been trialled in the management of proliferating IH. Timolol, a non-selective β-blocker used for the treatment of increased intra-ocular pressure, has been demonstrated to produce a clinically beneficial response when applied topically in the form of a 0.5% solution or gel. Topical timolol has been mostly used for localised, non-ulcerated superficial Ns with a mean diameter of less than 11.3 mm, or a volume of approximately 100 mm³. Timolol does not penetrate deeply into the hemangioma volume, and hence it is not useful in the management of larger haemangiomas (Guo et al., 2010, Arch. Opthalmol., 128, 255-256; Chan et al., 2013, 131, e1739-1747). Topical propranolol 1% ointments have also been trialled in small clinical studies of proliferating IH with good to modest response rates in approximately 75-90% (57-59% good response, 26-33% modest response, 10-15% no response) (Kunzi-Rapp. 2012, Pediatr. Dermatol. 29, 154-159; Xu et al., 2012, J. Am. Acad. Dermatol., 67, 1210-1213). However, in a randomised, double blind, placebo-controlled, multicentre study assessing the efficacy of a propranolol 1% gel in 81 patients, only 6 patients (14.6%) demonstrated a significant clinical response compared with 1 patient (2.6%) in the placebo group (Clinical Study Report V00400 GL 2 01 1A). This is in contrast to the aforementioned response rate of approximately 60% observed with orally administered propranolol.

### OBJECT

It is an object of the present invention to provide an improved method and/or composition for the treatment of hemangioma or at least to provide the public with a useful choice.

### STATEMENT OF INVENTION

In first aspect the invention provides a method for the treatment of a hemangioma in a subject, the method comprising at least the step of locally administering an ACEi to the hemangioma.

In a second aspect, the invention provides a method for the treatment of a hemangioma in a subject, the method comprising at least the step of locally administering an ACEi and a beta-blocker to the hemangioma.

In one embodiment, the beta-blocker is administered in a lower amount relative to the amount of ACEi administered. In one embodiment, the ratio of ACEi to beta-blocker administered is from approximately 1:1 to approximately 10:1. In other embodiments, the ratio of ACEi to beta-blocker administered is from approximately 2:1 to approximately 10:1, approximately 3:1 to approximately 9:1, approximately 4:1 to approximately 8:1, or approximately 5:1 to approximately 7:1. In other embodiments the ratio is approximately 1:1, approximately 2:1, approximately 3:1, approximately 4:1, approximately 5:1, approximately 6:1, approximately 7:1, approximately 8:1, approximately 9:1 or approximately 10:1.

In one embodiment, the ACEi and beta-blocker are administered simultaneously. In another embodiment, the ACEi and beta-blocker are administered sequentially in any order.

In a third embodiment, the invention provides a method for the treatment of a hemangioma in a subject, the method comprising at least the step of locally administering an ACEi and an ATIIR2 antagonist to the hemangioma.

In one embodiment, the ACEi and ATIIR2 antagonist are administered simultaneously. In another embodiment, the ACEi and ATIIR2 antagonist are administered sequentially in any order.

In a fourth embodiment, the invention provides a method for the treatment of a hemangioma in a subject, the method comprising at least the step of locally administering a beta-blocker and an ATIIR2 antagonist to the hemangioma.

In one embodiment, the beta-blocker and ATIIR2 antagonist are administered simultaneously. In another embodiment, the beta-blocker and ATIIR2 antagonist are administered sequentially in any order.

In a fifth embodiment, the invention provides a method for the treatment of a hemangioma in a subject, the method comprising at least the step of locally administering an ACEi, a beta-blocker and an ATIIR2 antagonist to the hemangioma.

In one embodiment, the ACEi, beta-blocker and ATIIR2 antagonist are administered simultaneously. In another embodiment, the ACEi, beta-blocker and ATIIR2 antagonist are administered sequentially in any order.

In one embodiment of the first to fifth aspects, two or more ACEi are administered. In another embodiment, two or more beta-blockers are administered. In another embodiment, two or more ATIIR2 antagonists are administered. The two or more ACEi, beta-blockers and ATIIR2 antagonists may be administered simultaneously or sequentially, in any order.

In one embodiment of the first to fifth aspects, the ACEi, beta-blocker and/or ATIIR2 antagonists are administered topically. In another embodiment of the first to fifth aspects, the ACEi, beta-blocker, and/or ATIIR2 antagonist are administered via local injection to the hemangioma.

In one embodiment of the first to fifth aspects, the ACEi is a prodrug. In a preferred embodiment, the ACEi is chosen from the group consisting of: Enalapril, Ramipril, Trandolapril, Cilazapril, Benazepril, Perindopril, Imidapril, Fosinopril, Zofenopril and Quinapril. In another embodiment, the ACEi is chosen from Captopril and Lisinopril.

In one embodiment of the first to fifth aspects, the beta-blocker is a non-selective beta-blocker.

In one embodiment of the first to fifth aspects, the method further comprises systemically administering at least one ACEi, at least one beta-blocker and/or at least one ATIIR2 antagonist to the subject. In one embodiment, the at least one ACEi, beta-blocker and/or ATIIR2 antagonist are administered orally. In one particular embodiment, at least one beta-blocker or at least one ACEi is administered.

In one particular embodiment of the first aspect, the method comprises locally administering an ACEi and systemically administering a beta-blocker. In another embodiment, the method comprises locally administering an ACEi and systemically administering an ACEi. In another embodiment, the method comprises locally administering an ACEi and systemically administering an ATIIR2 antagonist. In one embodiment, the agent(s) locally administered are administered topically and the agent(s) administered systemically are administered orally.

In one embodiment of the first aspect, the method comprises locally administering an ACE inhibitor and a beta-blocker, and systemically administering a beta-blocker. In another embodiment, the method comprises locally administering an ACEi and beta-blocker and systemically administering an ACEi. In another embodiment, the method comprises locally administering an ACEi and a beta-blocker and systemically administering an ATIIR2 antagonist. In one embodiment, the agent(s) locally administered are administered topically and the agent(s) administered systemically are administered orally.

In one embodiment of the fourth aspect, the method comprises locally administering an ATIIR2 antagonist and a beta-blocker, and systemically administering a beta-blocker. In another embodiment, the method comprises locally administering an ATIIR2 antagonist and a beta-blocker, and systemically administering an ACEi. In another embodiment, the method comprises locally administering an ATIIR2 antagonist and a beta-blocker, and systemically administering an ATIIR2. In one embodiment, the agent(s) locally administered are administered topically and the agent(s) administered systemically are administered orally.

In one embodiment of the third aspect, the method comprises locally administering an ACEi and an ATIIR2 antagonist, and systemically administering a beta-blocker. In another embodiment, the method comprises locally administering an ACEi and an ATIIR2 antagonist, and systemically administering an ACEi. In another embodiment, the method comprises locally administering an ACEi and an ATIIR2 antagonist, and systemically administering an ATIIR2. In one embodiment, the agent(s) locally administered are administered topically and the agent(s) administered systemically are administered orally.

In one embodiment of the first to fifth aspects, the method comprises locally administering an ACEi, a beta-blocker and an ATIIR2 antagonist, and systemically administering an ACEi, an ATIIR2 antagonist or a beta blocker. In one embodiment, the agent(s) locally administered are administered topically and the agent(s) administered systemically are administered orally.

In embodiments of the second to fifth aspects, the methods comprise locally administering: a non-selective beta-blocker and an ACEi in prodrug form; a non-selective beta-blocker and an ATIIR2 antagonist; or an ATIIR2 antagonist and an ACEi in prodrug form.

In embodiments of the second to fifth aspects, the method comprises locally administering:

R-propranolol and cilazapril; S-propranolol and cilazapril; RS-propranolol and cilazapril; R-propranolol and ramipril; S-propranolol and ramipril; RS-propranolol and ramipril; R-propranolol and trandolapril; S-propranolol and trandolapril; RS-propranolol and trandolapril; R-propranolol and enalapril; S-propranolol and enalapril; RS-propranolol and enalapril; R-propranolol and quinapril; S-propranolol and quinapril; RS-propranolol and quinapril; R-propranolol and benazepril; S-propranolol and benazepril; RS-propranolol and benazepril; R-propranolol and captopril; S-propranolol and captopril; RS-propranolol and captopril; R-timolol and cilazapril; S-timolol and cilazapril; RS-timolol and cilazapril; R-timolol and ramipril; S-timolol and ramipril; RS-timolol and ramipril; R-timolol and trandolapril; S-timolol and trandolapril; RS-timolol and trandolapril; R-timolol and enalapril; S-timolol and enalapril; RS-timolol and enalapril; R-timolol and quinapril; S-timolol and quinapril; RS-timolol and quinapril; R-timolol and benazepril; S-timolol and benazepril; RS-timolol and benazepril; R-timolol and captopril; S-timolol and captopril; RS-timolol and captopril; R-propranolol and EMA401; S-propranolol and EMA401; RS-propranolol and EMA401; R-timolol and EMA401; S-timolol and EMA401; RS-timolol and EMA401; R-propranolol and SMM02; S-propranolol and SMM02; RS-propranolol and SMM02; R-timolol and SMM02; S-timolol and SMM02; RS-timolol and SMM02; EMA401 and cilazapril; EMA401 and ramipril; EMA401 and trandolapril; EMA401 and enalapril; EMA401 and quinapril; EMA401 and benazepril; EMA401 and captopril; SMM02 and cilazapril; SMM02 and ramipril; SMM02 and trandolapril; SMM02 and quinapril; SMM02 and benazepril; or, SMM02 and captopril.

In certain embodiments of the second to fifth aspects, the methods comprise locally administering: R-timolol and cilazapril; S-timolol and cilazapril; R/S-timolol and cilazapril; R-propranolol and cilazapril; S-propranolol and cilazapril; R/S-propranolol and cilazapril; SMM02 and cilazapril; R-propranolol and SMM02; S-propranolol and SMM02; R/S-propranolol and SMM02; SMM02 and S-timolol; SMM02 and R-timolol; EMA401 and S-timolol; EMA401 and R-timolol; EMA401 and cilazapril; EMA401 and R-propranolol; EMA401 and S-propranolol; or, EMA401 and R/S-propranolol.

In a sixth aspect, the invention provides an ACEi, or the use of an ACEi, for the treatment of a hemangioma in a subject, wherein the ACEi is formulated for local administration to the hemangioma.

In a seventh aspect, the invention provides an ACEi and a beta-blocker, or the use of an ACEi and a beta-blocker, for the treatment of a hemangioma in a subject, wherein the ACEi and beta-blocker are formulated for local administration to the hemangioma. In one embodiment of the seventh aspect, the invention provides an ACEi for the treatment of hemangioma in a subject in combination with a beta blocker, wherein the ACEI and beta-blocker are formulated for local administration to the hemangioma. In one embodiment of the seventh aspect, the invention provides a beta-blocker for the treatment of hemangioma in a subject in combination with an ACEi, wherein the ACEI and beta-blocker are formulated for local administration to the hemangioma.

In one embodiment, the beta-blocker is used in a lower amount relative to the ACEi. In one embodiment, the ratio of ACEi to beta-blocker used is from approximately to approximately 10:1. In other embodiments, the ratio of ACEi to beta-blocker used is from approximately 2:1 to approximately 10:1, approximately 3:1 to approximately 9:1, approximately 4:1 to approximately 8:1 or approximately 5:1 to approximately 7:1. In other embodiments the ratio is approximately 1:1, approximately 2:1, approximately 3:1, approximately 4:1, approximately 5:1, approximately 6:1, approximately 7:1, approximately 8:1, approximately 9:1 or approximately 10:1.

In one embodiment, the ACEi and beta-blocker are used simultaneously. In another embodiment, they are used sequentially in any order.

In an eighth aspect, the invention provides an ACEi and an ATIIR2 antagonist, or the use of an ACEi and an ATIIR2 antagonist, for the treatment of a hemangioma in a subject, wherein the ACEi and ATIIR2 are formulated for local administration to the hemangioma. In one embodiment of the eighth aspect, the invention provides an ACEi for the treatment of hemangioma in a subject in combination with an ATIIR2 antagonist, wherein the ACEI and ATIIR2 antagonist are formulated for local administration to the hemangioma. In one embodiment of the eighth aspect, the invention provides an ATIIR2 antagonist for the treatment of hemangioma in a subject in combination with an ACEi, wherein the ACEI and ATIIR2 antagonist are formulated for local administration to the hemangioma.

In one embodiment, the ACEi and ATIIR2 are used simultaneously. In another embodiment, they are used sequentially in any order.

In a ninth aspect, the invention provides a beta-blocker and an ATIIR2 antagonist, or the use of a beta-blocker and an ATIIR2 antagonist, for the treatment of a hemangioma in a subject, wherein the beta-blocker and ATIIR2 are formulated for local administration to the hemangioma. In one embodiment of the ninth aspect, the invention provides an ATIIR2 antagonist for the treatment of hemangioma in a subject in combination with a beta-blocker, wherein the beta-blocker and ATIIR2 antagonist are formulated for local administration to the hemangioma. In one embodiment of the ninth aspect, the invention provides a beta-blocker for the treatment of hemangioma in a subject in combination with an ATIIR2 antagonist, wherein the beta-blocker and ATIIR2 antagonist are formulated for local administration to the hemangioma.

In one embodiment, the beta-blocker and ATIIR2 are used simultaneously. In another embodiment, they are used sequentially in any order.

In a tenth aspect, the invention provides an ACEi, a beta-blocker and an ATIIR2 antagonist, or the use of an ACEi, a beta-blocker and an ATIIR2 antagonist, for the treatment of a hemangioma in a subject, wherein the ACEi, beta-blocker and ATIIR2 antagonist are formulated for local administration to the hemangioma. In one embodiment of the tenth aspect, the invention provides an ACEi for the treatment of hemangioma in a subject in combination with a beta-blocker and an ATIIR2 antagonist, wherein the ACEi, beta-blocker and ATIIR2 antagonist are formulated for local administration to the hemangioma. In one embodiment of the tenth aspect, the invention provides an ATIIR2 antagonist for the treatment of hemangioma in a subject in combination with a beta-blocker and an ACEi, wherein the beta-blocker, ACEi and ATIIR2 antagonist are formulated for local administration to the hemangioma. In one embodiment of the tenth aspect, the invention provides a beta-blocker for the treatment of hemangioma in a subject in combination with an ATIIR2 antagonist and an ACEi, wherein the beta-blocker, ACEi and ATIIR2 antagonist are formulated for local administration to the hemangioma.

In one embodiment, the ACEi, beta-blocker and ATIIR2 antagonist are used simultaneously. In another embodiment, they are used sequentially in any order.

In one embodiment of the sixth to tenth aspects, two or more ACEi are used. In another embodiment, two or more beta-blockers are used. In another embodiment, two or more ATIIR2 antagonists are used. The two or more ACEi, beta-blockers and ATIIR2 antagonists may be used simultaneously or sequentially, in any order.

In one embodiment of the sixth to tenth aspects, the ACEi, beta-blocker and/or ATIIR2 antagonists are formulated for topical administration. In another embodiment of the sixth to tenth aspects, the ACEi, beta-blocker and/or ATIIR2 antagonist are formulated for local injection.

In one embodiment of the sixth to tenth aspects, the ACEi is a prodrug. In a preferred embodiment, the ACEi is chosen from the group consisting of: Enalapril, Ramipril, Trandolapril, Cilazapril, Benazepril, Perindopril, Imidapril, Fosinopril, Zofenopril and Quinapril. In another embodiment, the ACEi is chosen from Captopril and Lisinopril.

In one embodiment of the sixth to tenth aspects, the beta-blocker is a non-selective beta-blocker.

In one embodiment of the sixth to tenth aspects, the treatment further comprises systemically administering at least one ACEi, at least one beta-blocker and/or at least one ATIIR2 antagonist to the subject. In one embodiment, the at least one ACEi, beta-blocker and/or ATIIR2 antagonist are administered orally. In one particular embodiment, at least one beta-blocker or at least one ACEi is administered.

In one embodiment of the sixth aspect, the invention provides i) an ACEi and ii) an ACEi, a beta-blocker and/or an ATIIR2 antagonist, or the use of i) an ACEi and ii) an ACEi, a beta-blocker and/or an ATIIR2 antagonist, for the treatment of a hemangioma in a subject, wherein i) is formulated for local administration to the hemangioma and ii) is formulated for systemic administration to the subject (ie i) an ACEi for the treatment of a hemangioma in a subject in combination with ii) an ACEi, a beta-blocker and/or an ATIIR2 antagonist, wherein i) is formulated for local administration to the hemangioma and ii) is formulated for systemic administration to the subject).

In one particular embodiment of the sixth aspect, the invention provides i) an ACEi and ii) a beta-blocker, or the use of i) an ACEi and ii) a beta-blocker, for the treatment of hemangioma in a subject, wherein i) is formulated for local administration to the hemangioma and ii) is formulated for systemic administration to the subject. In another embodiment, the invention provides the use of i) an ACEi formulated for local administration to the hemangioma and ii) an ACEi formulated for systemic administration to the subject. In another embodiment, the invention provides the use of i) an ACEi formulated for local administration to the hemangioma and ii) an ATIIR2 formulated for systemic administration to the subject. In preferred embodiments, i) is formulated for topical administration and ii) is formulated for oral administration.

In one embodiment of the seventh aspect, the invention provides i) an ACEi and a beta-blocker and ii) an ACEi, a beta-blocker and/or an ATIIR2 antagonist, or the use of i) an ACEi and a beta-blocker and ii) an ACEi, a beta-blocker and/or an ATIIR2 antagonist, for the treatment of a hemangioma in a subject, wherein i) is formulated for local administration to the hemangioma and ii) is formulated for systemic administration to the subject (i.e. i) an ACEi and a beta-blocker for the treatment of a hemangioma in a subject in combination with ii) an ACEi, a beta-blocker and/or an ATIIR2 antagonist, wherein i) is formulated for local administration to the hemangioma and ii) is formulated for systemic administration to the subject). In one embodiment, the invention provides i) an ACEi and a beta-blocker and ii) an ACEi or a beta-blocker or an ATIIR2, or the use of i) an ACEi and a beta-blocker and ii) an ACEi or a beta-blocker or an ATIIR2, for the treatment of a hemangioma in a subject, wherein i) is formulated for local administration to the hemangioma and ii) is formulated for systemic administration to the subject. In preferred embodiments, i) is formulated for topical administration and ii) is formulated for oral administration.

In one embodiment of the eighth aspect, the invention provides of i) an ACEi and an ATIIR2 antagonist and ii) an ACEi, a beta-blocker and/or an ATIIR2 antagonist, or the use of i) an ACEi and a ATIIR2 and ii) an ACEi, a beta-blocker and/or an ATIIR2 antagonist, for the treatment of a hemangioma in a subject, wherein i) is formulated for local administration to the hemangioma and ii) is formulated for systemic administration to the subject (ie i) an ACEi and an ATIIR2 antagonist for the treatment of a hemangioma in a subject in combination with ii) an ACEi, a beta-blocker and/or an ATIIR2 antagonist, wherein i) is formulated for local administration to the hemangioma and ii) is formulated for systemic administration to the subject). In one embodiment, the invention provides i) an ACEi and a ATIIR2 and ii) an ACEi or a beta-blocker or an ATIIR2, or the use of i) an ACEi and a ATIIR2 and ii) an ACEi or a beta-blocker or an ATIIR2, for the treatment of a hemangioma in a subject, wherein i) is formulated for local administration to the hemangioma and ii) is formulated for systemic administration to the subject. In preferred embodiments, i) is formulated for topical administration and ii) is formulated for oral administration.

In one embodiment of the ninth aspect, the invention provides i) a beta-blocker and an ATIIR2 antagonist and ii) an ACEi, a beta-blocker and/or an ATIIR2 antagonist, or the use of i) a beta-blocker and an ATIIR2 and ii) an ACEi, a beta-blocker and/or an ATIIR2 antagonist, for the treatment of a hemangioma in a subject, wherein i) is formulated for local administration to the hemangioma and ii) is formulated for systemic administration to the subject (i.e. i) a beta-blocker and an ATIIR2 antagonist for the treatment of a hemangioma in a subject in combination with ii) an ACEi, a beta-blocker and/or an ATIIR2 antagonist, wherein i) is formulated for local administration to the hemangioma and ii) is formulated for systemic administration to the subject). In one embodiment, the invention provides i) a beta-blocker and an ATIIR2 and ii) an ACEi or a beta-blocker or an ATIIR2, or the use of i) a beta-blocker and an ATIIR2 and ii) an ACEi or a beta-blocker or an ATIIR2, for the treatment of a hemangioma in a subject, wherein i) is formulated for local administration to the hemangioma and ii) is formulated for systemic administration to the subject. In preferred embodiments, i) is formulated for topical administration and ii) is formulated for oral administration.

In one embodiment of the tenth aspect, the invention provides i) an ACEi, a beta-blocker and an ATIIR2 antagonist and ii) an ACEi, a beta-blocker and/or an ATIIR2 antagonist, or the use of i) an ACEi, a beta-blocker and an ATIIR2 antagonist and ii) an ACEi, a beta-blocker and/or an ATIIR2 antagonist, for the treatment of a hemangioma in a subject, wherein i) is formulated for local administration to the hemangioma and ii) is formulated for systemic administration to the subject (i.e. i) an ACEi, a beta-blocker and an ATIIR2 antagonist for the treatment of a hemangioma in a subject in combination with ii) an ACEi, a beta-blocker and/or an ATIIR2 antagonist, wherein i) is formulated for local administration to the hemangioma and ii) is formulated for systemic administration to the subject).

In one embodiment of the tenth aspect, the invention provides i) an ACEi, a beta-blocker and an ATIIR2 antagonist and ii) an ACEi or a beta-blocker or an ATIIR2 antagonist, or the use of i) an ACEi, a beta-blocker and an ATIIR2 antagonist and ii) an ACEi or a beta-blocker or an ATIIR2 antagonist, for the treatment of a hemangioma in a subject, wherein i) is formulated for local administration to the hemangioma and ii) is formulated for systemic administration to the subject. In preferred embodiments, i) is formulated for topical administration and ii) is formulated for oral administration.

In embodiments of the seventh to nineth aspects, there is provided: a non-selective beta-blocker and an ACEi in prodrug form; a non-selective beta-blocker and an ATIIR2 antagonist; an ATIIR2 antagonist and an ACEi in prodrug form, or the use of said combination, for the treatment of hemangioma by local administration.

In embodiments of the seventh to nineth aspects, there is provided: R-propranolol and cilazapril; S-propranolol and cilazapril; RS-propranolol and cilazapril; R-propranolol and ramipril; S-propranolol and ramipril; RS-propranolol and ramipril; R-propranolol and trandolapril; S-propranolol and trandolapril; RS-propranolol and trandolapril; R-propranolol and enalapril; S-propranolol and enalapril; RS-propranolol and enalapril; R-propranolol and quinapril; S-propranolol and quinapril; RS-propranolol and quinapril; R-propranolol and benazepril; S-propranolol and benazepril; RS-propranolol and benazepril; R-propranolol and captopril; S-propranolol and captopril; RS-propranolol and captopril; R-timolol and cilazapril; S-timolol and cilazapril; RS-timolol and cilazapril; R-timolol and ramipril; S-timolol and ramipril; RS-timolol and ramipril; R-timolol and trandolapril; S-timolol and trandolapril; RS-timolol and trandolapril; R-timolol and enalapril; S-timolol and enalapril; RS-timolol and enalapril; R-timolol and quinapril; S-timolol and quinapril; RS-timolol and quinapril; R-timolol and benazepril; S-timolol and benazepril; RS-timolol and benazepril; R-timolol and captopril; S-timolol and captopril; RS-timolol and captopril; R-propranolol and EMA401; S-propranolol and EMA401; RS-propranolol and EMA401; R-timolol and EMA401; S-timolol and EMA401; RS-timolol and EMA401; R-propranolol and SMM02; S-propranolol and SMM02; RS-propranolol and SMM02; R-timolol and SMM02; S-timolol and SMM02; RS-timolol and SMM02; EMA401 and cilazapril; EMA401 and ramipril; EMA401 and trandolapril; EMA401 and enalapril; EMA401 and quinapril; EMA401 and benazepril; EMA401 and captopril; SMM02 and cilazapril; SMM02 and ramipril; SMM02 and trandolapril; SMM02 and quinapril; SMM02 and benazepril; or, SMM02 and captopril, or the use of any one of the said combinations, for the treatment of hemangioma by local administration.

In embodiments of the seventh to nineth aspects, there is provided: R-timolol and cilazapril; S-timolol and cilazapril; RS-timolol and cilazapril; R-timolol and SMM02; S-timolol and SMM02; R/S-timolol and SMM02; R-timolol and EMA401; S-timolol and EMA401; R/S-timolol and EMA401; R-propranolol and cilazapril; S-propranolol and cilazapril; R/S-propranolol and cilazapril; SMM02 and cilazapril; EMA401 and cilazapril; R-propranolol and SMM02; R-propranolol and EMA401; S-propranolol and SMM02; S-propranolol and EMA401; R/S-propranolol and EMA401; or, R/S-propranolol and SMM02; or the use of any one of the said combinations, for the treatment of hemangioma by local administration.

In an eleventh aspect, the invention provides the use of an ACEi in the manufacture of a medicament for the treatment of a hemangioma in a subject, wherein the medicament is formulated for local administration to the hemangioma.

In a related aspect, the invention provides the use of an ACEi in the manufacture of a medicament for the treatment of a hemangioma in a subject, wherein the medicament is formulated for local administration to the hemangioma in combination with beta-blocker simultaneously or sequentially in any order. In a preferred embodiment, the beta-blocker is formulated for local administration.

In a related aspect, the invention provides the use of an ACEi in the manufacture of a medicament for the treatment of a hemangioma in a subject, wherein the medicament is formulated for local administration to the hemangioma in combination with an ATIIR2 antagonist simultaneously or sequentially in any order. In a preferred embodiment, the ATIIR2 antagonist is formulated for local administration.

In a related aspect, the invention provides the use of an ACEi in the manufacture of a medicament for the treatment of a hemangioma in a subject, wherein the medicament is formulated for local administration to the hemangioma in combination with an ATIIR2 antagonist and a beta-blocker simultaneously or sequentially in any order. In a preferred embodiment, the ATIIR2 antagonist and beta-blocker are formulated for local administration.

In a twelfth aspect, the invention provides the use of an ACEi and a beta-blocker in the manufacture of a medicament for the treatment of a hemangioma in a subject, wherein the medicament is formulated for local administration to the hemangioma.

In one embodiment, there is a lower amount of the beta-blocker in medicament relative to the ACEi inhibitor. In one embodiment, the ratio of ACEi to beta-blocker in the medicament is from approximately 1:1 to approximately 10:1. In other embodiments, the ratio of ACEi to beta-blocker in the medicament is from approximately 2:1 to approximately 10:1, approximately 3:1 to approximately 9:1, approximately 4:1 to approximately 8:1 or approximately 5:1 to approximately 7:1. In other embodiments the ratio is approximately 1:1, approximately 2:1, approximately 3:1, approximately 4:1, approximately 5:1, approximately 6:1, approximately 7:1, approximately 8:1, approximately 9:1 or approximately 10:1.

In a related aspect, the invention provides the use of a beta-blocker in the manufacture of a medicament for the treatment of a hemangioma in a subject, wherein the medicament is formulated for local administration to the hemangioma in combination with an ACEi simultaneously or sequentially in any order. In a preferred embodiment, the ACEi is formulated for local administration.

In a thirteenth aspect, the invention provides the use of an ACEi and an ATIIR2 antagonist in the manufacture of a medicament for the treatment of a hemangioma in a subject, wherein the medicament is formulated for local administration to the hemangioma.

In a related aspect, the invention provides the use of an ATIIR2 in the manufacture of a medicament for the treatment of a hemangioma in a subject, wherein the medicament is formulated for local administration to the hemangioma in combination with an ACEi simultaneously or sequentially in any order. In a preferred embodiment, the ACEi is formulated for local administration.

In a fourteenth aspect, the invention provides the use of a beta-blocker and an ATIIR2 antagonist in the manufacture of a medicament for the treatment of a hemangioma in a subject, wherein the medicament is formulated for local administration to the hemangioma.

In a related aspect, the invention provides the use of a beta-blocker in the manufacture of a medicament for the treatment of a hemangioma in a subject, wherein the medicament is formulated for local administration to the hemangioma in combination with an ATIIR2 antagonist simultaneously or sequentially in any order. The ATIIR2 antagonist may formulated for local or systemic administration, preferably local administration.

In a related aspect, the invention provides the use of an ATIIR2 antagonist in the manufacture of a medicament for the treatment of a hemangioma in a subject, wherein the medicament is formulated for local administration to the hemangioma in combination with a beta-blocker simultaneously or sequentially in any order. The beta-blocker may be formulated for local or systemic administration. In a preferred embodiment, the beta-blocker is formulated for local administration.

In a fifteenth aspect, the invention provides the use of an ACEi, a beta-blocker and an ATIIR2 antagonist in the manufacture of a medicament for the treatment of a hemangioma in a subject, wherein the medicament is formulated for local administration to the hemangioma.

In a related aspect, the invention provides the use of a beta-blocker in the manufacture of a medicament for the treatment of a hemangioma in a subject, wherein the medicament is formulated for local administration to the hemangioma in combination with an ACEi and an ATIIR2 antagonist simultaneously or sequentially in any order. In a preferred embodiment, the ACEi is formulated for local administration. In a preferred embodiment, the ATIIR2 is formulated for local administration.

In a related aspect, the invention provides the use of an ATIIR2 in the manufacture of a medicament for the treatment of a hemangioma in a subject, wherein the medicament is formulated for local administration to the hemangioma in combination with an ACEi and a beta-blocker simultaneously or sequentially in any order. In a preferred embodiment, the ACEi is formulated for local administration. In a preferred embodiment, the beta-blocker is formulated for local administration.

In one embodiment of the eleventh to fifteenth and their related aspects, the medicament comprises a combination of two or more ACEi. In another embodiment, the medicament comprises a combination of two or more beta-blockers. In another embodiment, the medicament comprises a combination of two or more ATIIR2 antagonists.

In one embodiment of the eleventh to fifteenth and their related aspects, the medicaments are formulated for topical administration. In another embodiment of the eleventh to fifteenth aspects, the medicaments are formulated for local injection.

In one embodiment of the eleventh to fifteenth and their related aspects, the ACEi is a prodrug. In a preferred embodiment, the ACEi is chosen from the group consisting of: Enalapril, Ramipril, Trandolapril, Cilazapril, Benazepril, Perindopril, Imidapril, Fosinopril, Zofenopriland Quinapril. In another embodiment, the ACEi is chosen from Captopril and Lisinopril.

In one embodiment of the eleventh to fifteenth and their related aspects, the beta-blocker is a non-selective beta-blocker.

In one embodiment of the eleventh to fifteenth and related aspects, the medicament is formulated for local administration in combination with systemic administration of an ACEi, beta-blocker and/or an ATIIR2 antagonist simultaneously or sequentially in any order.

In one embodiment of the eleventh to fifteenth and related aspects, the medicament is formulated for local administration in combination with systemic administration of an ACEi simultaneously or sequentially in any order. In another embodiment, the medicament is formulated for local administration in combination with systemic administration of a beta-blocker simultaneously or sequentially in any order. In another embodiment, the medicament is formulated for local administration in combination with systemic administration of an ATIIR2 antagonist simultaneously or sequentially in any order.

In certain embodiments of the eleventh to fifteenth and related aspects, where the combination of agents is referred to: the beta-blocker is a non-selective beta-blocker and the ACEi is in prodrug form; the beta-blocker is a non-selective beta-blocker and an ATIIR2 antagonist; or, the ACEi is in prodrug form and an ATIIR2 antagonist.

In certain embodiments of the eleventh to fifteenth and related aspects, where the use of a combination of agents is referred to: the beta-blocker is R-propranolol and the ACEi is cilazapril; the beta-blocker is S-propranolol and the ACEi is cilazapril; the beta-blocker is RS-propranolol and the ACEi is cilazapril; the beta-blocker is R-propranolol and the ACEi is ramipril; the beta-blocker is S-propranolol and the ACEi is ramipril; the beta-blocker is RS-propranolol and the ACEi is ramipril; the beta-blocker is R-propranolol and the ACEi is trandolapril; the beta-blocker is S-propranolol and the ACEi is trandolapril; the beta-blocker is RS-propranolol and the ACEi is trandolapril; the beta-blocker is R-propranolol and the ACEi is enalapril; the beta-blocker is S-propranolol and the ACEi is enalapril; the beta-blocker is RS-propranolol and the ACEi is enalapril; the beta-blocker is R-propranolol and the ACEi is quinapril; the beta-blocker is S-propranolol and the ACEi is quinapril; the beta-blocker is RS-propranolol and the ACEi is quinapril; the beta-blocker is R-propranolol and the ACEi is benazepril; the beta-blocker is S-propranolol and the ACEi is benazepril; the beta-blocker is RS-propranolol and the ACEi is benazepril; the beta-blocker is R-propranolol and the ACEi is captopril; the beta-blocker is S-propranolol and the ACEi is captopril; the beta-blocker is RS-propranolol and the ACEi is captopril; the beta-blocker is R-timolol and the ACEi is cilazapril; the beta-blocker is S-timolol and the ACEi is cilazapril; the beta-blocker is RS-timolol and the ACEi is cilazapril; the beta-blocker is R-timolol and the ACEi is ramipril; the beta-blocker is S-timolol and the ACEi is ramipril; the beta-blocker is RS-timolol and the ACEi is ramipril; the beta-blocker is R-timolol and the ACEi is trandolapril; the beta-blocker is S-timolol and the ACEi is trandolapril; the beta-blocker is RS-timolol and the ACEi is trandolapril; the beta-blocker is R-timolol and the ACEi is enalapril; the beta-blocker is S-timolol and the ACEi is enalapril; the beta-blocker is RS-timolol and the ACEi is enalapril; the beta-blocker is R-timolol and the ACEi is quinapril; the beta-blocker is S-timolol and the ACEi is quinapril; the beta-blocker is RS-timolol and the ACEi is quinapril; the beta-blocker is R-timolol and the ACEi is benazepril; the beta-blocker is S-timolol and the ACEi is benazepril; the beta-blocker is RS-timolol and the ACEi is benazepril; the beta-blocker is R-timolol and the ACEi is captopril; the beta-blocker is S-timolol and the ACEi is captopril; the beta-blocker is RS-timolol and the ACEi is captopril; the beta-blocker is R-propranolol and the ATIIR2 antagonist is EMA401; the beta-blocker is S-propranolol and the ATIIR2 antagonist is EMA401; the beta-blocker is RS-propranolol and the ATIIR2 antagonist is EMA401; the beta-blocker is R-timolol and the ATIIR2 antagonist is EMA401; the beta-blocker is S-timolol and the ATIIR2 antagonist is EMA401; the beta-blocker is RS-timolol and the ATIIR2 antagonist is EMA401; the beta-blocker is R-propranolol and the ATIIR2 antagonist is SMM02; the beta-blocker is S-propranolol and the ATIIR2 antagonist is SMM02; the beta-blocker is RS-propranolol and the ATIIR2 antagonist is SMM02; the beta-blocker is R-timolol and and the ATIIR2 antagonist is SMM02; the beta-blocker is S-timolol and the ATIIR2 antagonist is SMM02; the beta-blocker is RS-timolol and the ATIIR2 antagonist is SMM02; the ATIIR2 antagonist is EMA401 and the ACEi is cilazapril; the ATIIR2 antagonist is EMA401 and the ACEi is ramipril; the ATIIR2 antagonist is EMA401 and the ACEi is trandolapril; the ATIIR2 antagonist is EMA401 and the ACEi is enalapril; the ATIIR2 antagonist is EMA401 and the ACEi is quinapril; the ATIIR2 antagonist is EMA401 and the ACEi is benazepril; the ATIIR2 antagonist is EMA401 and the ACEi is captopril; the ATIIR2 antagonist is SMM02 and the ACEi is cilazapril; the ATIIR2 antagonist is SMM02 and the ACEi is ramipril; the ATIIR2 antagonist is SMM02 and the ACEi is trandolapril; the ATIIR2 antagonist is SMM02 and the ACEi is quinapril; the ATIIR2 antagonist is SMM02 and the ACEi is benazepril; or, the ATIIR2 antagonist is SMM02 and the ACEi is captopril.

In certain embodiments of the eleventh to fifteenth and related aspects, where the use of a combination of agents is referred to: the beta-blocker is R-timolol and the ACEi is cilazapril; the beta-blocker is S-timolol and the ACEi is cilazapril; the beta-blocker is RS-timolol and the ACEi is cilazapril; the beta-blocker is R-timolol and the ATIIR2 antagonist is SMM02; the beta-blocker is S-timolol and the ATIIR2 antagonist is SMM02; the beta blocker is RS-timolol and the ATIIR2 antagonist is SMM02; the beta-blocker is R-timolol and the ATIIR2 antagonist is EMA401; the beta-blocker is S-timolol and the ATIIR2 antagonist is EMA401; the beta-blocker is RS-timolol and the ATIIR2 antagonist is EMA401; the beta-blocker is R-propranolol and the ACEi is cilazapril; the beta-blocker is S-propranolol and the ACEi is cilazapril; the beta blocker is R/S-propranolol and the ACEi is cilazapril; the ATIIR2 antagonist is SMM02 and the ACEi is cilazapril; the ATIIR2 antagonist is EMA401 and the ACEi is cilazapril; the beta-blocker is R-propranolol and the ATIIR2 antagonist is SMM02; the beta-blocker is R-propranolol and the ATIIR2 antagonist is EMA401; the beta-blocker is S-propranolol and the ATIIR2 antagonist is SMM02; the beta-blocker is S-propranolol and the ATIIR2 antagonist is EMA401; the beta-blocker is R/S-propranolol and the ATIIR2 antagonist is SMM02; or, the beta-blocker is R/S-propranolol and the ATIIR2 antagonist is EMA401.

In a sixteenth aspect, the invention provides a method for the treatment of a hemangioma in a subject, the method comprising at least the step of systemically administering two or more of an ACEi, a beta-blocker and an ATIIR2 antagonist to a subject.

In one embodiment of the sixteenth aspect, the method comprises at least the step of systemically administering an ACEi and a beta-blocker. In other embodiments, the method comprises at least the step of systemically administering: an ACEi and an ATIIR2 antagonist; a beta-blocker and an ATIIR2 antagonist; or, an ACEi, a beta-blocker and an ATIIR2 antagonist.

In one embodiment of the sixteenth aspect, the ACEi, beta-blocker and ATIIR2 antagonist are administered simultaneously. In another embodiment, ACEi, beta-blocker and ATIIR2 antagonist are administered sequentially in any order.

In one embodiment of the sixteenth aspect, the ACEi, beta-blocker and/or ATIIR2 antagonist are administered orally.

In one embodiment of the sixteenth aspect, an ACEi is in prodrug form.

In one embodiment of the sixteenth aspect, a beta-blocker is a non-selective beta-blocker.

In certain embodiments of the sixteenth aspect, the methods comprise at least the step of systemically administering to a subject: a non-selective beta-blocker and an ACEi in prodrug form; a non-selective beta-blocker and an ATIIR2 antagonist; or, an ATIIR2 antagonist and an ACEi in prodrug form.

In certain embodiments of the sixteenth aspect, the methods comprise at least the step of systemically administering to a subject: R-propranolol and cilazapril; S-propranolol and cilazapril; RS-propranolol and cilazapril; R-propranolol and ramipril; S-propranolol and ramipril; RS-propranolol and ramipril; R-propranolol and trandolapril; S-propranolol and trandolapril; RS-propranolol and trandolapril; R-propranolol and enalapril; S-propranolol and enalapril; RS-propranolol and enalapril; R-propranolol and quinapril; S-propranolol and quinapril; RS-propranolol and quinapril; R-propranolol and benazepril; S-propranolol and benazepril; RS-propranolol and benazepril; R-propranolol and captopril; S-propranolol and captopril; RS-propranolol and captopril; R-timolol and cilazapril; S-timolol and cilazapril; RS-timolol and cilazapril; R-timolol and ramipril; S-timolol and ramipril; RS-timolol and ramipril; R-timolol and trandolapril; S-timolol and trandolapril; RS-timolol and trandolapril; R-timolol and enalapril; S-timolol and enalapril; RS-timolol and enalapril; R-timolol and quinapril; S-timolol and quinapril; RS-timolol and quinapril; R-timolol and benazepril; S-timolol and benazepril; RS-timolol and benazepril; R-timolol and captopril; S-timolol and captopril; RS-timolol and captopril; R-propranolol and EMA401; S-propranolol and EMA401; RS-propranolol and EMA401; R-timolol and EMA401; S-timolol and EMA401; RS-timolol and EMA401; R-propranolol and SMM02; S-propranolol and SMM02; RS-propranolol and SMM02; R-timolol and SMM02; S-timolol and SMM02; RS-timolol and SMM02; EMA401 and cilazapril; EMA401 and ramipril; EMA401 and trandolapril; EMA401 and enalapril; EMA401 and quinapril; EMA401 and benazepril; EMA401 and captopril; SMM02 and cilazapril; SMM02 and ramipril; SMM02 and trandolapril; SMM02 and quinapril; SMM02 and benazepril; or, SMM02 and captopril.

In certain embodiments of the sixteenth aspect, the methods comprise at least the step of systemically administering to a subject: R-timolol and cilazapril; S-timolol and cilazapril; RS-timolol and cilazapril; R-propranolol and cilazapril; S-propranolol and cilazapril; R/S-propranolol and cilazapril; SMM02 and cilazapril; EMA401 and cilazapril; R-propranolol and SMM02; S-propranolol and SMM02; R/S-propranolol and SMM02; R-propranolol and EMA401; S-propranolol and EMA401; RS-propranolol and EMA401; EMA401 and R-timolol; EMA401 and S-timolol; or, EMA401 and RS-timolol.

In one embodiment of the sixteenth aspect, the methods comprise administering two or more ACEi, two or more beta-blockers and/or two or more ATIIR2 antagonists.

In a seventeenth aspect, the invention provides two or more of an ACEi, a beta-blocker and an ATIIR2 antagonist, or the use of two or more of an ACEi, a beta-blocker and an ATIIR2 antagonist, for the treatment of a hemangioma in a subject, wherein the ACEi, beta-blocker and an ATIIR2 antagonist are formulated for systemic administration.

In one embodiment, the invention provides an ACEi for treatment of a hemangioma in a subject in combination with at least one of a beta-blocker and an ATIIR2 antagonist, wherein the ACEi, beta-blocker and ATIIR2 antagonist are formulated for systemic administration. In one embodiment, the invention provides an ATIIR2 antagonist for treatment of a hemangioma in a subject in combination with at least one of a beta-blocker and an ACEi, wherein the ACEi, beta-blocker and ATIIR2 antagonist are formulated for systemic administration. In one embodiment, the invention provides a beta-blocker for treatment of a hemangioma in a subject in combination with at least one of an ACEI and an ATIIR2 antagonist, wherein the ACEi, beta-blocker and ATIIR2 antagonist are formulated for systemic administration.

In one embodiment of the seventeenth aspect, an ACEi is in prodrug form.

In one embodiment of the seventeenth aspect, a beta-blocker is a non-selective beta-blocker.

In embodiments of the seventeenth aspect, there is provided: a non-selective beta-blocker and an ACEi in prodrug form; a non-selective beta-blocker and an ATIIR2 antagonist; an ACEi in prodrug form and an ATIIR2 antagonist, or the use of said combinations, for the treatment of hemangioma by systemic administration.

In embodiments of the seventeenth aspect, there is provided: R-propranolol and cilazapril; S-propranolol and cilazapril; RS-propranolol and cilazapril; R-propranolol and ramipril; S-propranolol and ramipril; RS-propranolol and ramipril; R-propranolol and trandolapril; S-propranolol and trandolapril; RS-propranolol and trandolapril; R-propranolol and enalapril; S-propranolol and enalapril; RS-propranolol and enalapril; R-propranolol and quinapril; S-propranolol and quinapril; RS-propranolol and quinapril; R-propranolol and benazepril; S-propranolol and benazepril; RS-propranolol and benazepril; R-propranolol and captopril; S-propranolol and captopril; RS-propranolol and captopril; R-timolol and cilazapril; S-timolol and cilazapril; RS-timolol and cilazapril; R-timolol and ramipril; S-timolol and ramipril; RS-timolol and ramipril; R-timolol and trandolapril; S-timolol and trandolapril; RS-timolol and trandolapril; R-timolol and enalapril; S-timolol and enalapril; RS-timolol and enalapril; R-timolol and quinapril; S-timolol and quinapril; RS-timolol and quinapril; R-timolol and benazepril; S-timolol and benazepril; RS-timolol and benazepril; R-timolol and captopril; S-timolol and captopril; RS-timolol and captopril; R-propranolol and EMA401; S-propranolol and EMA401; RS-propranolol and EMA401; R-timolol and EMA401; S-timolol and EMA401; RS-timolol and EMA401; R-propranolol and SMM02; S-propranolol and SMM02; RS-propranolol and SMM02; R-timolol and SMM02; S-timolol and SMM02; RS-timolol and SMM02; EMA401 and cilazapril; EMA401 and ramipril; EMA401 and trandolapril; EMA401 and enalapril; EMA401 and quinapril; EMA401 and benazepril; EMA401 and captopril; SMM02 and cilazapril; SMM02 and ramipril; SMM02 and trandolapril; SMM02 and quinapril; SMM02 and benazepril; or, SMM02 and captopril; or the use of any one of the said combinations, for the treatment of hemangioma by systemic administration.

In embodiments of the seventeenth aspect, there is provided: R-timolol and cilazapril; S-timolol and cilazapril; R/S-timolol and cilazapril; R-propranolol and cilazapril; S-propranolol and cilazapril; R/S-propranolol and cilazapril; SMM02 and cilazapril; R-propranolol and SMM02; R-propranolol and EMA401; S-propranolol and SMM02; S-propranolol and EMA401; R/S-propranolol and SMM02; R/S-propranolol and EMA401; R-timolol and SMM02; S-timolol and SMM02; R/S-timolol and SMM02; R-timolol and EMA401; S-timolol and EMA401; R/S-timolol and EMA401; cilazapril and EMA401; or the use of any one of the said combinations, for the treatment of hemangioma by systemic administration.

In an eighteenth aspect, the invention provides the use of two or more of an ACEi a beta-blocker and an ATIIR2 antagonist in the manufacture of a medicament for the treatment of a hemangioma in a subject, wherein the medicament is formulated for systemic administration.

In one embodiment of the seventeenth or eighteenth aspects, a combination of an ACEi and a beta-blocker is used. In other embodiments, a combination of: an ACEi and an ATIIR2 antagonist; a beta-blocker and an ATIIR2 antagonist; or, an ACEi, a beta-blocker and an ATIIR2 antagonist are used.

In one embodiment of the eighteenth aspect, an ACEi is in prodrug form.

In one embodiment of the eighteenth aspect, a beta-blocker is a non-selective beta-blocker.

In one embodiment of the seventeenth or eighteenth aspects, the ACEi, beta-blocker and ATIIR2 antagonist are formulated for simultaneous administration. In another embodiment, ACEi, beta-blocker and ATIIR2 antagonist are formulated for sequential administration in any order.

In one embodiment of the seventeenth or eighteenth aspects, the ACEi, beta-blocker and ATIIR2 antagonist are formulated for oral administration.

In certain embodiments of the eighteenth aspect, the invention provides the use of: a non-selective beta-blocker and an ACEi in prodrug form; a non-selective beta-blocker and an ATIIR2 antagonist; or, an ATIIR2 antagonist and an ACEi in prodrug form; in the manufacture of a medicament for the treatment of hemangioma in a subject, wherein the medicament is formulated for systemic administration.

In certain embodiments of the eighteenth aspect, the invention provides the use of: R-propranolol and cilazapril; S-propranolol and cilazapril; RS-propranolol and cilazapril; R-propranolol and ramipril; S-propranolol and ramipril; RS-propranolol and ramipril; R-propranolol and trandolapril; S-propranolol and trandolapril; RS-propranolol and trandolapril; R-propranolol and enalapril; S-propranolol and enalapril; RS-propranolol and enalapril; R-propranolol and quinapril; S-propranolol and quinapril; RS-propranolol and quinapril; R-propranolol and benazepril; S-propranolol and benazepril; RS-propranolol and benazepril; R-propranolol and captopril; S-propranolol and captopril; RS-propranolol and captopril; R-timolol and cilazapril; S-timolol and cilazapril; RS-timolol and cilazapril; R-timolol and ramipril; S-timolol and ramipril; RS-timolol and ramipril; R-timolol and trandolapril; S-timolol and trandolapril; RS-timolol and trandolapril; R-timolol and enalapril; S-timolol and enalapril; RS-timolol and enalapril; R-timolol and quinapril; S-timolol and quinapril; RS-timolol and quinapril; R-timolol and benazepril; S-timolol and benazepril; RS-timolol and benazepril; R-timolol and captopril; S-timolol and captopril; RS-timolol and captopril; R-propranolol and EMA401; S-propranolol and EMA401; RS-propranolol and EMA401; R-timolol and EMA401; S-timolol and EMA401; RS-timolol and EMA401; R-propranolol and SMM02; S-propranolol and SMM02; RS-propranolol and SMM02; R-timolol and SMM02; S-timolol and SMM02; RS-timolol and SMM02; EMA401 and cilazapril; EMA401 and ramipril; EMA401 and trandolapril; EMA401 and enalapril; EMA401 and quinapril; EMA401 and benazepril; EMA401 and captopril; SMM02 and cilazapril; SMM02 and ramipril; SMM02 and trandolapril; SMM02 and quinapril; SMM02 and benazepril; or, SMM02 and captopril; in the manufacture of a medicament for the treatment of hemangioma in a subject, wherein the medicament is formulated for systemic administration.

In certain embodiments of the eighteenth aspect, the invention provides the use of: R-timolol and cilazapril; S-timolol and cilazapril; R/S-timolol and cilazapril; R-timolol and SMM02; S-timolol and SMM02; R/S-timolol and SMM02; R-timolol and EMA401; S-timolol and EMA401; R/S-timolol and EMA401; R-propranolol and cilazapril; S-propranolol and cilazapril; R/S-propranolol and cilazapril; SMM02 and cilazapril; EMA401 and cilazapril; R-propranolol and SMM02; S-propranolol and SMM02; R/S-propranolol and SMM02; R-propranolol and EMA401; S-propranolol and EMA401; or, R/S-propranolol and EMA401; in the manufacture of a medicament for the treatment of a hemangioma in a subject, wherein the medicament is formulated for systemic administration.

In one embodiment of the seventeenth or eighteenth aspects, two or more ACEi, two or more beta-blockers and/or two or more ATIIR2 antagonists are used.

In a nineteenth aspect, the invention provides a composition comprising a combination of an ACEi and a beta-blocker, wherein the composition is suitable for local administration to a hemangioma. In one embodiment, the composition is suitable for topical administration. In one embodiment, the composition comprises a lower amount of beta-blocker relative to the amount of ACEi.

In one embodiment, the ratio of ACEi to beta-blocker in the composition is from approximately 1:1 to approximately 10:1. In other embodiments, the ratio of ACEi to beta-blocker in the composition is from approximately 2:1 to approximately 10:1, approximately 3:1 to approximately 9:1, approximately 4:1 to approximately 8:1 or approximately 5:1 to approximately 7:1. In other embodiments the ratio is approximately 1:1, approximately 2:1, approximately 3:1, approximately 4:1, approximately 5:1, approximately 6:1, approximately 7:1, approximately 8:1, approximately 9:1 or approximately 10:1.

In one embodiment, the composition comprises an ACEi in prodrug form. In one embodiment, the ACEi is chosen from the group consisting of: Enalapril, Ramipril, Trandolapril, Cilazapril, Benazepril, Quinapril, Perindopril, Imidapril, Fosinopril and Zofenopril. In another embodiment, the ACEi is chosen from the group consisting of: Captopril and Lisinopril.

In one embodiment, the composition comprises a non-selective beta-blocker.

In a twentieth aspect, the invention provides a composition comprising an ACEi and an ATIIR2 antagonist, wherein the composition is suitable for local administration to a hemangioma. In one embodiment, the composition is suitable for topical administration.

In a twenty first aspect, the invention provides a composition comprising a beta-blocker and an ATIIR2 antagonist, wherein the composition is suitable for local administration to a hemangioma. In one embodiment, the composition is suitable for topical administration.

In a twenty second aspect, the invention provides a composition comprising an ACEi, a beta-blocker and an ATIIR2 antagonist, wherein the composition is suitable for local administration to a hemangioma. In one embodiment, the composition is suitable for topical administration.

In certain embodiments of the nineteenth to twenty second aspect, a composition comprises: a non-selective beta-blocker and an ACEi in prodrug form; a non-selective beta-blocker and an ATIIR2 antagonist; or, an ATIIR2 antagonist and an ACEi in prodrug form.

In certain embodiments of the nineteenth to twenty second aspect, a composition comprises: R-propranolol and cilazapril; S-propranolol and cilazapril; RS-propranolol and cilazapril; R-propranolol and ramipril; S-propranolol and ramipril; RS-propranolol and ramipril; R-propranolol and trandolapril; S-propranolol and trandolapril; RS-propranolol and trandolapril; R-propranolol and enalapril; S-propranolol and enalapril; RS-propranolol and enalapril; R-propranolol and quinapril; S-propranolol and quinapril; RS-propranolol and quinapril; R-propranolol and benazepril; S-propranolol and benazepril; RS-propranolol and benazepril; R-propranolol and captopril; S-propranolol and captopril; RS-propranolol and captopril; R-timolol and cilazapril; S-timolol and cilazapril; RS-timolol and cilazapril; R-timolol and ramipril; S-timolol and ramipril; RS-timolol and ramipril; R-timolol and trandolapril; S-timolol and trandolapril; RS-timolol and trandolapril; R-timolol and enalapril; S-timolol and enalapril; RS-timolol and enalapril; R-timolol and quinapril; S-timolol and quinapril; RS-timolol and quinapril; R-timolol and benazepril; S-timolol and benazepril; RS-timolol and benazepril; R-timolol and captopril; S-timolol and captopril; RS-timolol and captopril; R-propranolol and EMA401; S-propranolol and EMA401; RS-propranolol and EMA401; R-timolol and EMA401; S-timolol and EMA401; RS-timolol and EMA401; R-propranolol and SMM02; S-propranolol and SMM02; RS-propranolol and SMM02; R-timolol and SMM02; S-timolol and SMM02; RS-timolol and SMM02; EMA401 and cilazapril; EMA401 and ramipril; EMA401 and trandolapril; EMA401 and enalapril; EMA401 and quinapril; EMA401 and benazepril; EMA401 and captopril; SMM02 and cilazapril; SMM02 and ramipril; SMM02 and trandolapril; SMM02 and quinapril; SMM02 and benazepril; or, SMM02 and captopril.

In certain embodiments of the nineteenth to twenty second aspect, a composition comprises: R-timolol and cilazapril; S-timolol and cilazapril; R/S-timolol and cilazapril; R-timolol and SMM02; S-timolol and SMM02; R/S-timolol and cilazapril; R-timolol and EMA401; S-timolol and EMA401; R/S-timolol and EMA401; R-propranolol and cilazapril; S-propranolol and cilazapril; R/S-propranolol and cilazapril; SMM02 and cilazapril; EMA401 and cilazapril; R-propranolol and SMM02; S-propranolol and SMM02; R/S-propranolol and SMM02; R-propranolol and EMA401; S-propranolol and EMA401; or, R/S-propranolol and EMA401.

In a twenty third aspect, the invention provides a method for the treatment of a hemangioma in a subject, the method comprising at least the step of locally administering an ATIIR2 antagonist to the hemangioma. In one embodiment, the ATIIR2 antagonist is administered topically.

In one embodiment of the twenty third aspect, the method further comprises administering a beta-blocker and/or an ACEi.

In one embodiment of the twenty third aspect, the beta-blocker and/or ACEi are administered locally to the hemangioma. In another embodiment, the beta-blocker and/or ACEi are administered systemically. In one embodiment, the ATIIR2 antagonist and the beta-blocker and/or ACEi are administered simultaneously. In another embodiment, the ATIIR2 antagonist and beta-blocker and/or ACEi are administered sequentially in any order.

In one embodiment of the twenty third aspect, two or more ATIIR2 antagonists are administered. In another embodiment, two or more beta-blockers are administered. In another embodiment, two or more ACEi are administered. The two or more ACEi, beta-blockers and ATIIR2 antagonists may be administered simultaneously or sequentially, in any order.

In a twenty fourth aspect, the invention provides an ATTIIR2 antagonist, or the use of an ATIIR2 antagonist, for the treatment of a hemangioma in a subject, wherein the ATIIR2 antagonist is formulated for local administration to the hemangioma. In one embodiment, the ATIIR2 antagonist is formulated for topical administration.

In one embodiment of the twenty fourth aspect, the ATIIR2 antagonist is for treatment of a hemangioma in a subject in combination with a beta-blocker and/or an ACEi. In one embodiment, the beta-blocker and/or ACEi are administered locally to the hemangioma. In another embodiment, the beta-blocker and/or ACEi are administered systemically. In one embodiment, the ATIIR2 antagonist and the beta-blocker and/or ACEi are administered simultaneously. In another embodiment, the ATIIR2 antagonist and beta-blocker and/or ACEi are administered sequentially in any order.

In one embodiment of the twenty fourth aspect, two or more ATIIR2 antagonists are used. In another embodiment, two or more beta-blockers are used. In another embodiment, two or more ACEi are used. The two or more ACEi, beta-blockers and ATIIR2 antagonists may be administered simultaneously or sequentially, in any order.

In a twenty fifth aspect, the invention provides the use of an ATIIR2 antagonist in the manufacture of a medicament for the treatment of a hemangioma in a subject, wherein the medicament is formulated for local administration to the hemangioma. In one embodiment, the medicament is formulated for topical administration.

In one embodiment of the twenty fifth aspect, the medicament is formulated for local administration to the hemangioma in combination with an ACEi and/or a beta-blocker simultaneously or sequentially in any order. In a preferred embodiment, the ACEi and/or beta-blocker are formulated for local administration. In another embodiment, the ACEi and/or beta-blocker are formulated for systemic administration.

In one embodiment of the twenty fifth aspect, the medicament comprises two or more ATIIR2 antagonists.

In one embodiment of the twenty fifth aspect, the medicament comprises one or more ACEi and/or one or more beta-blocker.

In one embodiment of the twenty fifth aspect, the medicament is formulated for local administration in combination with two or more ACEi and/or two or more beta-blockers.

In one embodiment of the twenty third to twenty fifth aspects, the ACEi is in prodrug form.

In one embodiment of the twenty third to twenty fifth aspects, the beta-blocker is a non-selective beta-blocker.

In one embodiment of the first to nineteenth and twenty third to twenty fifth aspects and related aspects, the hemangioma is infantile hemangioma. In another embodiment, the hemangioma is a proliferating hemangioma.

In one embodiment the beta-blocker is chosen from the group consisting of: propranolol, timolol, pindolol, sotalol, and atenolol. In one embodiment of the first to nineteenth aspects, the beta-blocker is a combination of enantiomers. In one embodiment, the beta-blocker is a combination of enantiomers comprising a higher proportion of the (R)-enantiomer. In one embodiment, the beta-blocker is substantially in the S-enantiomer form. In one embodiment, the beta-blocker is substantially in the R-enantiomer form. In a preferred embodiment, the beta-blocker is chosen from the group consisting of: R-timolol, S-timolol, R/S-timolol, R-propranolol, S-propranolol, R/S-propranolol.

In one embodiment of the first to twenty fifth aspects, the ATIIR2 antagonist is chosen from the group consisting of: EMA401, SMM02 (L-159,686); PD-123,319, PD-121,981, PD-126,055, L-161,638 (sodium salt), and L-163,579. In one embodiment of the first to twenty fifth aspects, the ATIIR2 antagonist is chosen from the group consisting of: EMA401 and SMM02.

The invention may also be said broadly to consist in the parts, elements and features referred to or indicated in the specification of the application, individually or collectively, in any and all combinations of two or more of said parts, elements or features, and where specific integers are mentioned herein which have known equivalents in the art to which the invention relates, such known equivalents are deemed to be incorporated herein as if individually set forth.

### FIGURES

These and other aspects of the invention, which should be considered in all its novel aspects, will become apparent from the following description, which is given by way of example only, with reference to the accompanying figures:

FIG. 1: Effect of an ACE inhibitor (cilazapril) and a beta blocker (timolol) on inhibiting haemangioma cell proliferation in vitro after a single 100 μM dose.

FIG. 2: Effect of ACE inhibitor (cilazapril) combined with a beta blocker (timolol) at different ratios on the inhibtion of haemangioma cell proliferation in vitro. Graph legend: Left bars: 24 hours; Middle bars: 48 hours; Right bars: 72 hours.

FIG. 3: The effect of a beta blocker (timolol) on the inhibition of proliferating infantile hemangioma cells derived from multiple patients in vitro (n=5).

FIG. 4: The effect of an ACE inhibitor (cilazapril) on the inhibition of proliferating infantile hemangioma cells derived from multiple patients in vitro (n=5).

FIG. 5: The effect of an AT2 receptor inhibitor (EMA401) on the inhibition of proliferating infantile hemangioma cells derived from multiple patients in vitro (n=5).

FIG. 6: The effect of an AT2 receptor inhibitor (EMA401) on the inhibition of proliferating infantile hemangioma cells derived from multiple patients (raw absorbance data) in vitro (n=5).

FIG. 7: The effect of an AT2 receptor inhibitor (SMM02) on the inhibition of proliferating infantile hemangioma cells derived from multiple patients in vitro (n=5).

FIG. 8: The effect of an AT2 receptor inhibitor (SMM02) on the inhibition of proliferating infantile hemangioma cells derived from multiple patients (raw absorbance data) in vitro (n=5).

FIG. 9: The effect of an AT1 receptor inhibitor (losartan) on the inhibition of proliferating infantile hemangioma cells derived from multiple patients in vitro (n=5).

FIG. 10: Change in cellular proliferation and effect of experimental adjuvants (solvents) on hemangioma cells (control experiment) in vitro (n=5).

FIG. 11: Effect of an ACE inhibitor (cilazapril) combined with a beta blocker (timolol) at different ratios on the inhibition of proliferating infantile hemangioma cells derived from multiple patients in vitro (n=5).

FIG. 12: Effect of a beta blocker (timolol) combined with an AT2 receptor inhibitor (EMA401) at different ratios on the inhibition of proliferating infantile hemangioma cells derived from multiple patients in vitro (n=5).

FIG. 13: Effect of a beta blocker (timolol) combined with an AT2 receptor inhibitor (SMM02) at different ratios on the inhibition of proliferating infantile hemangioma cells derived from multiple patients in vitro (n=5).

FIG. 14: Effect of an ACE inhibitor (cilazapril) combined with an AT2 receptor inhibitor (EMA401) at different ratios on the inhibition of proliferating infantile hemangioma cells derived from multiple patients in vitro (n=5).

FIG. 15: Effect of an ACE inhibitor (cilazapril) combined with an AT2 receptor inhibitor (SMM02) at different ratios on the inhibition of proliferating infantile hemangioma cells derived from multiple patients in vitro (n=5).

FIG. 16: The effect of beta blockers (timolol and propranolol) and ACE inhibitors (cilazapril) on the inhibition of proliferating haemangioma cells as single agents in vitro. Timolol (25A, B) and propranolol (25C, D) investigated as single enantiomers (S-timolol, R-timolol, S-propranolol and R-propranolol) and as a racemic mixture of enantiomers (R/S-propranolol) (25E).

FIG. 17: The effect of single enantiomers of timolol (beta blocker) in combination with cilazapril (ACE inhibitor) on the inhibition of proliferating haemangioma cells in vitro.

FIG. 18: The effect of single enantiomers of propranolol (beta blocker) or its racemic mixture in combination with cilazapril (ACE inhibitor) on the inhibition of proliferating haemangioma cells in vitro.

FIG. 19: Dose-response of renin-angiotensin system inhibitors as single agents against multiple proliferating hemangioma cell lines derived from different patient biopsies (n=9) after 72 hours of administration.

FIG. 20: The effect of an AT2 receptor inhibitor (SMM02) in combination with an ACE inhibitor (cilazapril) on the inhibition of multiple proliferating hemangioma cell lines (n=9) after 72 hours of administration.

FIG. 21: The effect of an ACE inhibitor (cilazapril) in combination with a beta blocker (R-propranolol) on the inhibition of multiple proliferating hemangioma cell lines (n=9) after 72 hours of administration.

FIG. 22: Average inhibition for the combination of cilazapril and R-propranolol on multiple proliferating hemangioma cell lines after 72 hours (n=9; error represents standard error of the mean).

FIG. 23: The effect of an ACE inhibitor (cilazapril) in combination with a beta blocker (S-propranolol) on the inhibition of multiple proliferating hemangioma cell lines (n=9) after 72 hours of administration.

FIG. 24: Average inhibition for the combination of cilazapril and S-propranolol on multiple proliferating hemangioma cell lines after 72 hours (n=9; error represents standard error of the mean).

FIG. 25: The effect of an AT2 receptor antagonist (SMM02) in combination with a beta blocker (R-propranolol) on the inhibition of multiple proliferating hemangioma cell lines (n=9) after 72 hours of administration.

FIG. 26: The effect of an AT2 receptor antagonist (SMM02) in combination with a beta blocker (S-propranolol) on the inhibition of multiple proliferating hemangioma cell lines (n=9) after 72 hours of administration.

FIG. 27: Distribution of Combination Index (CI) values across primary hemangioma cell lines (n=9) arising from the combination of an AT2 receptor antagonist (SMM02) and an ACE inhibitor (cilazapril) demonstrating on average a synergistic effect (CI<1) after 72 hours of administration.

FIG. 28: Distribution of Combination Index (CI) values across primary hemangioma cell lines (n=9) arising from the combination of an ACE inhibitor (cilazapril) and a beta blocker (R-propranolol) demonstrating on average a synergistic effect (CI<1) after 72 hours of administration.

FIG. 29: Distribution of Combination Index (CI) values across primary hemangioma cell lines (n=9) arising from the combination of an ACE inhibitor (cilazapril) and a beta blocker (S-propranolol) demonstrating on average a synergistic effect (CI<1) after 72 hours of administration.

FIG. 30: Distribution of Combination Index (CI) values across primary hemangioma cell lines (n=9) arising from the combination of an AT2 receptor antagonist (SMM02) and a beta blocker (R-propranolol) demonstrating on average a synergistic effect (CI<1) after 72 hours of administration.

FIG. 31: Distribution of Combination Index (CI) values across primary hemangioma cell lines (n=9) arising from the combination of an AT2 receptor antagonist (SMM02) and a beta blocker (S-propranolol) demonstrating on average a synergistic effect (CI<1) after 72 hours of administration.

FIG. 32: Effect of different beta blockers on the inhibition of primary hemangioma cells biopsied during the proliferating phase.

FIG. 33: Effect of different ACE inhibitors on the inhibition of primary hemangioma cells biopsied during the proliferating phase.

FIG. 34: (A) Custom-built Franz-diffusion cell apparatus used for in vitro skin penetration screening in combination with Strat-M membranes. (B) Example of membrane penetration of a CF solution alone (pH 7.4) (diamond), and a dilute suspension of CF-containing liposomes (<1 mM) (square). (C) Example of membrane permeation of a 0.5% CF Emulgel formulation.

FIG. 35: Preparation and characterisation of a lisinopril emulsion gel. A) Scalable route to preparation of the emulsion formulation. B) Photograph of lisinopril emulgel. C) Light microscopy of the emulgel formulation. Scale bar=5 μm. D) Freeze fracture transmission electron microscopy of the emulgel formulation. Scale bar=2 μm.

FIG. 36: Scalable production of liposomes. (A) Custom-built large volume liposome extruder. (B) Dynamic light scattering data demonstrating liposome size distribution after one, five, and ten extrusions.

FIG. 37: Preparation and characterisation of deformable liposome gel. (A) Route to scalable production of the deformable gel formulation. (B) Photograph of the final deformable liposome gel. (C) Freeze fracture transmission electron microscopy revealing deformable liposomes (light arrows) embedded within the hydrogel matrix (dark arrow).

FIG. 38: Fluorescence assay for the sensitive quantification of lisinopril in Franz diffusion experiments. (A) Reaction between lisinopril and fluorescamine to form a highly fluorescent conjugate. (B) Reproducible linear relationship between fluorescence intensity and lisinopril concentration.

FIG. 39: Permeation experiments. (A) Photograph of custom-built Franz diffusion cell containing a carboxyfluorescein containing liposome gel for visual clarity. (B) Permeation of lisinopril from the lisinopril emulgel formulation (circles) and deformable liposome (SQH) formulation (squares).

FIG. 40: (A) Propranolol hydrochloride standard curve determined by UV-Vis spectroscopy with an absorbance maxima of 288 nm. (B) Permeation of propranolol through skin-model membranes using a 1% propranolol/cyclodextrin emulsion gel formulation. (C) Permeation of propranolol through skin-model membranes using a 1% propranolol cream formulation. (D) Comparison between the permeation of propranolol (circles) and lisinopril (squares) through skin-model membranes using a 1% emulsion gel formulation.

### PREFERRED EMBODIMENTS

The following is a description of the invention, including preferred embodiments thereof, given in general terms. The invention is further elucidated from the disclosure given under the heading “Examples” herein below, which provides experimental data supporting the invention, specific examples of various aspects of the invention, and means of performing the invention.

Following extensive studies, the inventors have surprisingly found that local inhibition of ACE activity within a hemangioma microenvironment by administration of an ACE inhibitor is an effective mode of treatment with a superior long-term safety profile compared to the current preferred treatment standard; oral administration of a beta-blocker. This is contrary to various reports that beta-blockers offer superior treatment outcomes to ACEi.

The inventors have also found that the use of a combination of an ACEi and a beta-blocker applied locally to a hemangioma provides an unexpected synergistic effect, providing another alternative, and potentially superior, treatment option for hemangiomas. Due to the synergistic nature of the combination, the inventors contemplate that this option may be particularly useful for treating rebound hemangiomas, harder-to-treat hemangiomas that do not completely resolve with propranolol therapy alone, or as a dose-sparing front-line therapy that lowers the risk of systemic side effects.

Without wishing to be bound by theory, the inventors contemplate that the enhanced inhibitory effect of local administration of an ACE inhibitor in combination with a beta blocker may arise from the inhibition of multiple proliferation mechanisms involved in hemangioma; for example, the inhibition of the beta-1 and beta-2 adrenergic receptors associated with haemangioma stem cells, inhibition of the SOX18 transcription factor, depression of angiotensin II production and consequently decreased angiotensin II type 2 receptor stimulation, and the modulation of various other currently unknown targets.

In addition, the inventors have identified that the use of a combination of a beta-blocker and an angiotensin II type 2 receptor (ATIIR2) antagonist or a combination of an ACEi and ATIIR2 antagonist applied locally to a hemangioma provides yet further alternative treatment options for the treatment of hemangiomas. Again, the inventors contemplate that this may offer improved treatment outcomes compared to the use of a single type of compound alone, as they target multiple proliferation mechanisms involved in hemangioma.

The inventors have surprisingly found that using the combinations of agents described herein to treat primary hemangioma cells derived from individual patient biopsies during the proliferating phase produces a more consistent level of inhibition between patient samples (n=9). In contrast, a greater degree of variation in the response of these patient-derived cell lines is observed when treated with individual agents. Based on these results, the inventors contemplate that at least local treatment of hemangiomas with combinations of inhibitors targeting different aspects of the renin-angiotensin system (for example, ACEi, ATIIR2 antagonists and/or beta-blockers as described herein) may produce more consistent and clinically superior outcomes than treatment with the individual agents. The use of such combinations may also allow for lower doses of the relevant agent(s) to be administered to a subject (compared to the use of each single agent), with the potential benefit of lowering the risk of any adverse side effects that may be associated with such agents.

Further, the inventors have found that ACEi of particular use in the treatment of hemangioma via local administration include those in prodrug form. The was unexpected as it is understood in the art that ACEi prodrugs require activation through hepatic biotransformation, which suggests that systemic administration would be the only effective means of delivery.

The inventors have also surprisingly found that there is little difference between the ability of R-beta blockers and S-beta blockers to inhibit proliferating hemangioma cells, noting the individual isomers were as effective alone as the racemic mixture which is currently prescribed for the treatment of proliferating infantile hemangioma requiring systemic therapy. This is a significant finding as the R-enantiomers of beta-blockers (such as timolol and propranolol) do not exert blood pressure effects to the same degree as the S-enantiomers. Accordingly, the inventors propose the use of R-enantiomers of beta-blockers in hemangioma treatment regimens may allow for effective treatment while lowering or minimizing the risk of negative side effects which may be associated with the use of S-enantiomers and/or racemic mixtures of beta blockers.

Based on data generated, the inventors also contemplate that non-selective beta-blockers may be more effective in the treatment of hemangiomas than selective beta-blockers.

The inventors also contemplate that the local administration of an ATIIR2 antagonist may provide an effective treatment option for hemangiomas. Their research demonstrates that use of an ATIIR2 as a single agent is effective at lower doses compared to the use of an ACEi or a beta-blocker. Without wishing to be bound by theory, the inventors contemplate that ATIIR2 is abundantly expressed during infancy and likely plays a role in early growth and development. Clinical development of the first selective ATIIR2 antagonist was recently discontinued for systemic toxicity after long term use, and systemic side effects have previously been reported with the use of topical beta blockers. Thus, the inventors anticipate that treating patients with low dose, locally administered ATIIR2 antagonists will be substantially devoid of side effects (or at least have lower side effects) relative to systemically administered ATIIR2 antagonists, BB's and ACEi's, and mitigate the complications commonly associated with these therapies. Furthermore, the inventors have unexpectedly found that low doses of ATIIR2 antagonists produce consistently high levels of inhibition when treating proliferating hemangioma cell lines derived from individual patient biopsies in two independent investigations (Example 3: n=5 cell lines, and Example 6: n=9 cell lines). In contrast, a greater degree of variation in the response of these patient-derived cell lines when treated with BBs or ACEis as single agents. Based on these results, the inventors contemplate that local treatment of hemangiomas with ATIIR2 antagonists are likely to produce more consistent and clinically superior outcomes than treatment with BBs or ACEis as single agents.

The inventors have also identified differences in the relative activity and/or skin diffusion rates of ACEi and beta-blockers, informing novel dosages and treatment regimens for hemangioma.

The inventors contemplate the invention provides a first-in-line medicament/method for early treatment or intervention in the progression of hemangioma, for example, to prevent the lesion from growing or becoming physically or cosmetically disfiguring, ulcerated, or psychologically disturbing, from causing functional disability, for example blindness, and to prevent the formation of a fibro-fatty residuum. Where a lesion is not severe, the inventors believe a locally (preferably topically) applied ACE inhibitor or ATIIR2 antagonist can be used to effectively manage, control or prevent the growth of the lesion. In cases where a faster involution is required or it would otherwise be useful to augment the treatment of a hemangioma, the inventors contemplate the use of combination therapies to treat, manage or control the growth of the lesion: in one embodiment, an ACE inhibitor combined with a β-blocker; in another embodiment an ACE inhibitor combined with an ATIIR2 antagonist; in another embodiment, an ACE inhibitor combined with an ATIIR2 antagonist and a beta-blocker; in another embodiment, a beta-blocker combined and an ATIIR2 antagonist. In other embodiments, where the growth of a lesion is very severe, or the treatment response to local administration is lower or slower than desired, local administration in accordance with the invention may be combined with systemic administration of one or more agent of use in the treatment of hemangioma (for example, an ACEi, beta-blocker and/or ATIIR2 antagonist).

While the inventors' results indicate that there is benefit in using some form of local treatment of hemangiomas using an ACEi, or an ATIIR2 antagonist, or combinations of an ACEi, a beta-blocker and/or an ATIIR2 antagonist, they contemplate that such combinations may be equally useful for the treatment of hemangiomas by systemic routes, such as oral administration.

### Definitions

Reference to “treatment” herein should be taken broadly to include controlling, inhibiting, preventing or slowing the growth of a hemangioma, controlling or reducing the size of a hemangioma, ameliorating one or more symptoms associated with hemangioma, and/or improving the physical appearance of a hemangioma. It should not be taken to mean that a subject is treated until total recovery (such as complete removal or involution of the hemangioma), although that may be preferred.

A “subject” should be taken to include reference to any animal. In preferred embodiments, the subject is a mammal, more preferably a human. In other embodiments, the subject is a dog, cat or a horse.

“Local” administration should be taken to mean direct administration to the hemangioma (including reference to administration to the site of, or tissue proximal to (for example overlying), the hemangioma), in contrast to systemic administration. As will readily be appreciated from the nature of the different types of hemangioma and the context of the invention described herein (including the preferred method of topical delivery), “direct” administration to a hemangioma will be understood to include local administration to [the/a] tissue within which the hemangioma is situated, or which the hemangioma is proximal to. Skilled persons in the art will readily appreciate suitable local administration means. However, by way of example only, they may include (i) localised injection of the hemangioma or [a/the] proximal tissue and (ii) application of a medicament to a surface of the hemangioma or the surface of a tissue within which the hemangioma is situated or is proximal to (i.e. topical administration, for example to the skin). In particular embodiments of the methods of the invention, topical administration is preferred.

“Systemic” administration should be taken broadly to include any administration means which delivers an agent to the circulatory system of a subject. Systemic administration includes enteral and parenteral administration means. Skilled persons will readily appreciate suitable systemic administration means. In one particular embodiment of the methods of the invention, oral administration is preferred.

The phrase “pharmaceutically acceptable carrier, diluent and/or excipient” refers to any useful carriers, excipients, and diluents which are nontoxic to the cell or animal to which a composition is administered at the dosages and concentrations used. “Carriers, diluents and/or excipients” include but are not limited to fillers, colouring agents, preservatives, stabilising agents, bulking agents, agents which help control release of active agents, agents which enhance delivery, binders, solvents, emulsifiers, suspending agents, lubricants, agents which alter viscosity of the composition, and moisturisers.

In the context of the invention an “effective amount” of an agent to be administered to an animal is an amount necessary to at least partly attain a desired response.

Reference may be made herein to a beta-blocker being in the R-enantiomer form. This should not be taken to mean that the R-beta-blocker is necessarily 100% pure, although this may be preferred (within the limits of detection). Some tolerance for levels of another enantiomer may be tolerated.

The same is the case for references to 5-enantiomer forms. In some cases, the beta-blocker may comprise a racemic mixture, in preferred embodiments having at least a predominance of the R-enantiomer.

The term “ATIIR2 antagonist” is defined later herein. The alternative terms AT2 receptor inhibitor and AT2 receptor antagonist may be used herein interchangeably with ATIIR2 antagonist.

Unless the context clearly indicates otherwise, reference to the singular shall include reference to the plural. For example, reference to administering an ACEi should be understood to include reference to administering two or more ACEi. The same is the case for reference to ATIIR2 antagonists and beta-blockers.

Reference may be made herein to methods of the invention comprising administering a “combination” of two or more compounds (for example, ramipril and propranolol). Such references should not be taken to mean that the compounds are necessarily combined in a single composition or administered simultaneously (unless it is clear from the context that this is so). Such references are simply used to explain that two or more compounds are administered. Although, in certain embodiments the compounds may be combined in a single composition or administered simultaneously.

The alternative spellings hemangioma and haemangioma may be used interchangeably herein.

Reference may be made herein to administration of one or more active agent being administered in separate compositions. Where this occurs, it should be appreciated that the agent(s) may be provided in the form of a kit.

Methods

The invention provides methods for the treatment of hemangiomas by administering an effective amount of one or more active agent (eg. ACEi, ATIIR2 antagonist, beta-blocker) as described herein. In certain embodiments, the hemangioma is one which is present within, under or on the surface of the skin or a mucosal membrane of a subject. In certain embodiments, the hemangioma is chosen from the group comprising: infantile hemangioma, cutaneous hemangioma, capillary hemangioma, cavernous hemangioma, retinal hemangioma (including for example, retinal cavernous hemangioma, retinal capillary hemangioma, choroidal hemangioma, cavernous hemangioma of the orbit) and/or periocular hemangioma.

In a preferred embodiment, the methods of the invention are directed to the treatment of infantile hemangiomas (in one preferred embodiment, infantile capillary hemangioma). Infantile hemangiomas exhibit a characteristic evolution, consisting of an early rapid proliferative phase, followed by a slow spontaneous and prolonged (for example, 5 to 10 years) involution phase. Infantile hemangiomas usually become visible on the skin from approximately 4 to 6 weeks from birth. In some cases, infantile hemangiomas will not fully involute. Infantile hemangioma is also characterised by the expression of a homogenous group of immunohistochemical markers including GLUT1 (glucose transporter 1), which is a surface protein expressed by erythrocytes and the endothelium of infantile hemangiomas.

In certain preferred embodiments, the methods of the invention are directed to the treatment of proliferating hemangiomas; i.e. hemangiomas in a state or phase of growth or proliferation. Such hemangiomas are typically characterised by rapid spontaneous growth of the hemangioma lesion. By way of example, in infantile hemangioma, the proliferative phase usually occurs within the period from birth to approximately four to six weeks. The rapid rate of growth is characteristically beyond the growth rate of the infant, thereby differentiating it from vascular malformations that grow commensurate with the infant. By treating a hemangioma in this proliferative phase or state, the methods of the invention can be used to prevent further growth of the hemangioma and concomitant damage that may occur as a result of that growth.

The methods of the invention may be useful for the treatment of hemangiomas of any size and volume. While the inventors contemplate the methods of the invention being useful for the treatment of large or developed hemangiomas, in preferred embodiments the hemangioma is treated while it is relatively small, early in its development, to prevent further growth and the risks associated with such growth. In one embodiment, the methods of the invention may be useful for the treatment of hemangiomas having a volume of from at least approximately 0.01 cm³.

Local Administration

In one embodiment, the invention provides a method for the treatment of a hemangioma in a subject, the method comprising at least the step of locally administering an ACEi to the hemangioma. In one embodiment, the method comprises at least the step of locally administering an ACEi and a beta-blocker to the hemangioma. In another embodiment, the method comprises at least the step of locally administering an ACEi and a ATIIR2 antagonist to the hemangioma. In another embodiment, the method comprises at least the step of locally administering an ACEi, a beta-blocker and an ATIIR2 antagonist to the hemangioma. In another embodiment, the method comprises as least the step of locally administering a beta-blocker and an ATIIR2 antagonist to the hemangioma.

Local administration may occur by any appropriate means, including for example, topical administration and/or local injection. In a preferred embodiment, the ACEi, ACEi and beta-blocker, ACEi and ATIIR2 antagonist, beta-blocker and ATIIR2 antagonist or the ACEi, beta-blocker and ATIIR2 antagonist are administered topically at the site of the hemangioma.

In another embodiment, the invention provides a method for the treatment of a hemangioma in a subject, the method comprising at least the step of locally administering an ATIIR2 antagonist to the hemangioma. In one embodiment, the ATIIR2 antagonist is administered topically.

In certain embodiments, a combination of two or more ACEi are administered, a combination of two or more beta-blockers are administered, and/or a combination of two or more ATIIR2 antagonists are administered.

Where two or more agents (for example an ACEi and a beta-blocker, an ACEi and ATIIR2 antagonist, a beta-blocker and ATIIR2 antagonist, an ACEi, a beta-blocker and an ATIIR2 antagonist, or two or more ACEi, two or more beta-blockers or two or more ATIIR2 antagonists, and combinations thereof) are administered locally, they may be administered simultaneously or sequentially, in any order. The agent(s) may be administered in a single composition or in separate compositions.

In one embodiment, an ACEi and a beta-blocker are administered simultaneously. In another embodiment, an ACEi and a beta-blocker are administered sequentially in any order. In one embodiment, one or more ACEi is administered followed by one or more beta-blocker. In another embodiment, one or more beta-blocker is administered followed by one or more ACEi.

In one embodiment an ACEi and an ATIIR2 antagonist are administered simultaneously. In another embodiment, an ACEi and an ATIIR2 antagonist are administered sequentially in any order. In one embodiment, one or more ACEi is administered followed by one or more ATIIR2 antagonist. In another embodiment, one or more ATIIR2 antagonist is administered followed by one or more ACEi.

In one embodiment, a beta-blocker and an ATIIR2 antagonist are administered simultaneously. In one embodiment, a beta-blocker and an ATIIR2 antagonist are administered sequentially in any order. In one embodiment, one or more ATIIR2 antagonist is administered followed by one or more beta-blocker. In another embodiment, one or more beta-blocker is administered followed by one or more ATIIR2 antagonist.

In one embodiment, an ACEi, a beta-blocker and an ATIIR2 antagonist are administered simultaneously. In one embodiment, an ACEi, a beta-blocker and an ATIIR2 antagonist are administered sequentially in any order.

In certain embodiments a non-selective beta-blocker and an ACEi in prodrug form; a non-selective beta-blocker and an ATIIR2 antagonist; or an ATIIR2 antagonist and an ACEi in prodrug form; are administered, simultaneously or sequentially in any order.

In certain embodiments: R-propranolol and cilazapril; S-propranolol and cilazapril; RS-propranolol and cilazapril; R-propranolol and ramipril; S-propranolol and ramipril; RS-propranolol and ramipril; R-propranolol and trandolapril; S-propranolol and trandolapril; RS-propranolol and trandolapril; R-propranolol and enalapril; S-propranolol and enalapril; RS-propranolol and enalapril; R-propranolol and quinapril; S-propranolol and quinapril; RS-propranolol and quinapril; R-propranolol and benazepril; S-propranolol and benazepril; RS-propranolol and benazepril; R-propranolol and captopril; S-propranolol and captopril; RS-propranolol and captopril; R-timolol and cilazapril; S-timolol and cilazapril; RS-timolol and cilazapril; R-timolol and ramipril; S-timolol and ramipril; RS-timolol and ramipril; R-timolol and trandolapril; S-timolol and trandolapril; RS-timolol and trandolapril; R-timolol and enalapril; S-timolol and enalapril; RS-timolol and enalapril; R-timolol and quinapril; S-timolol and quinapril; RS-timolol and quinapril; R-timolol and benazepril; S-timolol and benazepril; RS-timolol and benazepril; R-timolol and captopril; S-timolol and captopril; RS-timolol and captopril; R-propranolol and EMA401; S-propranolol and EMA401; RS-propranolol and EMA401; R-timolol and EMA401; S-timolol and EMA401; RS-timolol and EMA401; R-propranolol and SMM02; S-propranolol and SMM02; RS-propranolol and SMM02; R-timolol and SMM02; S-timolol and SMM02; RS-timolol and SMM02; EMA401 and cilazapril; EMA401 and ramipril; EMA401 and trandolapril; EMA401 and enalapril; EMA401 and quinapril; EMA401 and benazepril; EMA401 and captopril; SMM02 and cilazapril; SMM02 and ramipril; SMM02 and trandolapril; SMM02 and quinapril; SMM02 and benazepril; or, SMM02 and captopril; are administered, simultaneously or sequentially in any order.

In certain embodiments R-timolol and cilazapril; S-timolol and cilazapril; R/S-timolol and cilazapril; R-propranolol and cilazapril; S-propranolol and cilazapril; R/S-propranolol and cilazapril; SMM02 and cilazapril; EMA401 and cilazapril; R-timolol and SMM02; S-timolol and SMM02; R/S-timolol and SMM02; R-timolol and EMA401; S-timolol and EMA401; R/S-timolol and EMA401; R-propranolol and SMM02; S-propranolol and SMM02; R/S-propranolol and SMM02; R-propranolol and EMA401; S-propranolol and EMA401; or, R/S-propranolol and EMA401; are administered, simultaneously or sequentially in any order.

Combination Local and Systemic Administration

In one embodiment, the methods of the invention further comprise systemically administering an ACEi, a beta-blocker and/or an ATIIR2 antagonist to the subject (ie in addition to local administration).

Systemic administration may occur by any appropriate means. However, in one preferred embodiment, at least one ACEi, at least one beta-blocker and/or at least one ATIIR2 antagonist are administered orally. In another embodiment, at least one ACEi, at least one beta-blocker and/or at least one ATIIR2 antagonist are administered by way of injection; for example, subcutaneous, intramuscular or intravenous injection.

In one particular embodiment, the method comprises locally administering an ACEi and systemically administering a beta-blocker. In another embodiment, the method comprises locally administering an ACEi and systemically administering an ACEi. In another embodiment, the method comprises locally administering an ACEi and systemically administering an ATIIR2 antagonist. In one embodiment, the agent(s) locally administered are administered topically at the site of the hemangioma and the agent(s) administered systemically are administered orally.

In one embodiment, the method comprises locally administering an ACE inhibitor and a beta-blocker, and systemically administering a beta-blocker. In another embodiment, the method comprises locally administering an ACEi and beta-blocker and systemically administering an ACEi. In another embodiment, the method comprises locally administering an ACE inhibitor and a beta-blocker, and systemically administering an ATIIR2 antagonist. In one embodiment, the agent(s) locally administered are administered topically at the site of the hemangioma and the agent(s) administered systemically are administered orally.

In one embodiment, the method comprises locally administering an ATIIR2 antagonist and a beta-blocker, and systemically administering a beta-blocker. In another embodiment, the method comprises locally administering an ATIIR2 antagonist and a beta-blocker, and systemically administering an ACEi. In one embodiment, the method comprises locally administering an ATIIR2 antagonist and a beta-blocker, and systemically administering an ATIIR2 antagonist. In one embodiment, the agent(s) locally administered are administered topically at the site of the hemangioma and the agent(s) administered systemically are administered orally.

In one embodiment, the method comprises locally administering an ACEi and an ATIIR2 antagonist, and systemically administering a beta-blocker. In another embodiment, the method comprises locally administering an ACEi and an ATIIR2 antagonist, and systemically administering an ACEi. In another embodiment, the method comprises locally administering an ACEi and an ATIIR2 antagonist, and systemically administering an ATIIR2 antagonist. In one embodiment, the agent(s) locally administered are administered topically at the site of the hemangioma and the agent(s) administered systemically are administered orally.

In one embodiment, the method comprises locally administering an ACEi, a beta-blocker and an ATIIR2 antagonist, and systemically administering an ACEi, an ATIIR2 antagonist or a beta blocker. In one embodiment, the agent(s) locally administered are administered topically at the site of the hemangioma and the agent(s) administered systemically are administered orally.

In another embodiment, the method comprises locally administering an ATIIR2 antagonist and systemically administering an ACEi, an ATIIR2 antagonist and/or a beta blocker. In one embodiment, the local administration is topical administration and the systemic administration is oral administration.

In certain embodiments: a non-selective beta-blocker and an ACEi in prodrug form; a non-selective beta-blocker and an ATIIR2 antagonist; or, an ACEi in prodrug form and an ATIIR2 antagonist are administered simultaneously or sequentially in any order. In one embodiment, one agent is administered locally and the other is administered systemically.

In certain embodiments: R-propranolol and cilazapril; S-propranolol and cilazapril; RS-propranolol and cilazapril; R-propranolol and ramipril; S-propranolol and ramipril; RS-propranolol and ramipril; R-propranolol and trandolapril; S-propranolol and trandolapril; RS-propranolol and trandolapril; R-propranolol and enalapril; S-propranolol and enalapril; RS-propranolol and enalapril; R-propranolol and quinapril; S-propranolol and quinapril; RS-propranolol and quinapril; R-propranolol and benazepril; S-propranolol and benazepril; RS-propranolol and benazepril; R-propranolol and captopril; S-propranolol and captopril; RS-propranolol and captopril; R-timolol and cilazapril; S-timolol and cilazapril; RS-timolol and cilazapril; R-timolol and ramipril; S-timolol and ramipril; RS-timolol and ramipril; R-timolol and trandolapril; S-timolol and trandolapril; RS-timolol and trandolapril; R-timolol and enalapril; S-timolol and enalapril; RS-timolol and enalapril; R-timolol and quinapril; S-timolol and quinapril; RS-timolol and quinapril; R-timolol and benazepril; S-timolol and benazepril; RS-timolol and benazepril; R-timolol and captopril; S-timolol and captopril; RS-timolol and captopril; R-propranolol and EMA401; S-propranolol and EMA401; RS-propranolol and EMA401; R-timolol and EMA401; S-timolol and EMA401; RS-timolol and EMA401; R-propranolol and SMM02; S-propranolol and SMM02; RS-propranolol and SMM02; R-timolol and SMM02; S-timolol and SMM02; RS-timolol and SMM02; EMA401 and cilazapril; EMA401 and ramipril; EMA401 and trandolapril; EMA401 and enalapril; EMA401 and quinapril; EMA401 and benazepril; EMA401 and captopril; SMM02 and cilazapril; SMM02 and ramipril; SMM02 and trandolapril; SMM02 and quinapril; SMM02 and benazepril; or, SMM02 and captopril; are administered simultaneously or sequentially in any order. In one embodiment, one agent is administered locally and the other is administered systemically.

In certain embodiments: R-timolol and cilazapril; S-timolol and cilazapril; R/S-timolol and cilazapril; R-timolol and SMM02; S-timolol and SMM02; R/S-timolol and SMM02; R-timolol and EMA401; S-timolol and EMA401; R/S-timolol and EMA401; R-propranolol and cilazapril; S-propranolol and cilazapril; R/S-propranolol and cilazapril; SMM02 and cilazapril; EMA401 and cilazapril; R-propranolol and SMM02; S-propranolol and SMM02; R/S-propranolol and SMM02; R-propranolol and EMA401; S-propranolol and EMA401; or, R/S-propranolol and EMA401; are administered simultaneously or sequentially in any order. In one embodiment, one agent is administered locally and the other is administered systemically.

In certain embodiments, two or more ACEi are administered, two or more beta-blockers are administered, and/or two or more ATIIR2 antagonists are administered systemically and/or locally, as the case may be.

Systemic Administration

In other aspects, the invention provides methods for the treatment of a hemangioma in a subject, the methods comprising at least the step of systemically administering two or more of an ACEi, a beta-blocker and an ATIIR2 antagonist to a subject. In a preferred embodiment, the method comprises at least the step of orally administering an ACEi and a beta-blocker. It should be appreciated that a combination of two or more of each type of active agent may be used in the methods of this aspect of the invention. In addition, the active agents may be administered simultaneously or sequentially in any order.

Systemic administration may occur by any appropriate means. However, in a preferred embodiment, the active agents are formulated for oral administration. In one embodiment, they are formulated in a solid oral dosage form. In an alternative embodiment, they are formulated in a liquid oral dosage form. This form is particularly preferred for paediatric applications.

In another embodiment, the active agents are formulated for administration by way of injection; for example, subcutaneous, intramuscular or intravenous injection. In other embodiments, the active agents are formulated so that they can be delivered via a drug delivery device, such as a transdermal patch, for example. In other embodiments, the active agents are formulated for administration by inhalation.

In certain embodiments: a non-selective beta-blocker and an ACEi in prodrug form; a non-selective beta-blocker and an ATIIR2 antagonist; or, an ACEi in prodrug form and an ATIIR2 antagonist; are administered systemically, simultaneously or sequentially in any order.

In certain embodiments: R-propranolol and cilazapril; S-propranolol and cilazapril; RS-propranolol and cilazapril; R-propranolol and ramipril; S-propranolol and ramipril; RS-propranolol and ramipril; R-propranolol and trandolapril; S-propranolol and trandolapril; RS-propranolol and trandolapril; R-propranolol and enalapril; S-propranolol and enalapril; RS-propranolol and enalapril; R-propranolol and quinapril; S-propranolol and quinapril; RS-propranolol and quinapril; R-propranolol and benazepril; S-propranolol and benazepril; RS-propranolol and benazepril; R-propranolol and captopril; S-propranolol and captopril; RS-propranolol and captopril; R-timolol and cilazapril; S-timolol and cilazapril; RS-timolol and cilazapril; R-timolol and ramipril; S-timolol and ramipril; RS-timolol and ramipril; R-timolol and trandolapril; S-timolol and trandolapril; RS-timolol and trandolapril; R-timolol and enalapril; S-timolol and enalapril; RS-timolol and enalapril; R-timolol and quinapril; S-timolol and quinapril; RS-timolol and quinapril; R-timolol and benazepril; S-timolol and benazepril; RS-timolol and benazepril; R-timolol and captopril; S-timolol and captopril; RS-timolol and captopril; R-propranolol and EMA401; S-propranolol and EMA401; RS-propranolol and EMA401; R-timolol and EMA401; S-timolol and EMA401; RS-timolol and EMA401; R-propranolol and SMM02; S-propranolol and SMM02; RS-propranolol and SMM02; R-timolol and SMM02; S-timolol and SMM02; RS-timolol and SMM02; EMA401 and cilazapril; EMA401 and ramipril; EMA401 and trandolapril; EMA401 and enalapril; EMA401 and quinapril; EMA401 and benazepril; EMA401 and captopril; SMM02 and cilazapril; SMM02 and ramipril; SMM02 and trandolapril; SMM02 and quinapril; SMM02 and benazepril; or, SMM02 and captopril; are administered systemically, simultaneously or sequentially in any order.

In certain embodiments: R-timolol and cilazapril; S-timolol and cilazapril; R/S-timolol and cilazapril; R-timolol and SMM02; S-timolol and SMM02; R/S-timolol and SMM02; R-timolol and EMA401; S-timolol and EMA401; R/S-timolol and EMA401; R-propranolol and cilazapril; S-propranolol and cilazapril; R/S-propranolol and cilazapril; SMM02 and cilazapril; EMA401 and cilazapril; R-propranolol and EMA401; S-propranolol and EMA401; R/S-propranolol and EMA401; R-propranolol and SMM02; S-propranolol and SMM02; or, R/S-propranolol and SMM02; are administered systemically, simultaneously or sequentially in any order.

It should be appreciated that the methods for the treatment of hemangioma in accordance with this aspect of the invention may be combined with the methods for local (eg. topical) administration according to other aspects of the invention as described herein before.

ACE Inhibitors

In certain embodiments of the invention, at least one ACE inhibitor (ACEi) is administered to a subject. An ACEi is any agent which is capable of selectively or non-selectively inhibiting, blocking, or at least decreasing the activity of angiotensin converting enzyme within a hemangioma lesion and includes ACEi prodrugs. Unless the context requires otherwise, reference to an ACEi herein should be taken to include reference to any pharmaceutically acceptable salt thereof. For the avoidance of doubt, reference to an ACEi herein is intended to include reference to stereoisomers thereof. A mixture of two or more ACEi's may be used in the methods of the invention.

Examples of ACE inhibitors of use in the invention include, but are not limited to, sulfhydryl-containing agents such as captopril, zofenopril, zofenoprilat, dicarboxylate-containing agents such as enalapril, enalaprilat, Ramipril, ramiprilat, quinapril, quinaprilat, perindopril, perindoprilat, lisinopril, benazepril, benazeprilat, imidapril, imidaprilat, trandolapril, trandolaprilat, cilazapril, cilazaprilat, phosphonate-containing agents such as fosinopril and fosinoprilat, or any other natural or synthetic agent with inhibitory activity against angiotensin converting enzyme.

In a preferred embodiment, the ACEi of the invention is in the form of a prodrug. In certain preferred embodiments, the ACEi is chosen from the group consisting of: Enalapril, Ramipril, Trandolapril, Cilazapril, Benazepril, Perindopril, Imidapril, Fosinopril, Zofenopril and Quinapril or a pharmaceutically acceptable salt of any one thereof. In another preferred embodiment, the ACEi are chosen from the group consisting of ramipril, trandolapril, cilazapril and quinapril or a pharmaceutically acceptable salt of any one thereof. In another embodiment, the ACEi is chosen from Captopril and Lisinopril or a pharmaceutically acceptable salt thereof. In another embodiment, the ACEi is a long-acting ACEi chosen from the group consisting of cilazapril, ramipril, trandolapril, and benazepril or a pharmaceutically acceptable salt of any one thereof.

Table 1 provides examples of a number of ACE inhibitors which may be useful in the methods of the invention, along with information on their physical, chemical and biological properties.

TABLE 1
Examples of ACE inhibitors and their chemical, physical, and biological properties.

| Name | Captopril | Enalapril | Ramipril | Trandolapril |
|---|---|---|---|---|
| Structure | 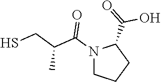 | 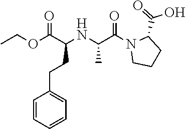 [SMILES: CCOC(=O)[C@H](CCc1ccccc1)N[C@@H](C)C(=O)N1CCC[C@H]1C(=O)O] | 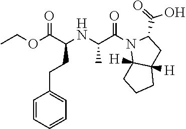 [SMILES: CCOC(=O)[C@H](CCc1ccccc1)N[C@@H](C)C(=O)N1[C@H](C(=O)O)C[C@@H]2CCC[C@@H]21] | 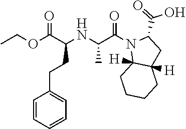 [SMILES: CCOC(=O)[C@H](CCc1ccccc1)N[C@@H](C)C(=O)N1[C@H](C(=O)O)C[C@@H]2CCCC[C@@H]21] |
| Type | Non-prodrug | Prodrug | Prodrug | Prodrug |
| Molecular | 217.29 | 376.45 | 416.51 | 430.53 |
| Weight | | | | |
| Log P | 0.34 | 0.07 | 2.9 | 3.5 |
| pKa | 3.7, 9.8 | 2.97, 5.35 | 3.75, 5.2 | 3.8, 5.2 |
| ACE IC50 | 6 nM | 240 nM (enalapril vs | 5 nM (Ramipril vs | 2.5 nM (Trandolapril |
| | | aorta ACE); 50 μM | brush border ACE). | vs aorta ACE). |
| | | (enalapril vs human | Complete ACE | (1.35 nM |
| | | renal ACE). | inhibition lasting 15 | Trandolaprilat vs |
| | | (35 nM enalaprilat vs | days after single 10 | aorta ACE) |
| | | human renal ACE) | μM dose. | |
| | | 140 nM (enalapril vs | | |
| | | guinea pig serum | | |
| | | ACE) | | |
| | | 3.1 nM (enalaprilat vs | | |
| | | guinea pig serum | | |
| | | ACE). | | |
| Solubility | 130 mg/mL | 16.4 mg/mL | 3.5 mg/L | — |
| Half Life | 1.9 hours | 11 hours (enalaprilat) | 2-4 hours (Ramipril) | 6 hours |
| | Short Acting | Long acting | 13-17 hours | (trandolapril) |
| | | | (Ramiprilat) | 10 hours |
| | | | Long acting | (trandolaprilat) |
| | | | | Long acting |
| Name | Lisinopril | Cilazapril | Benazepril | Quinapril |
| Structure | 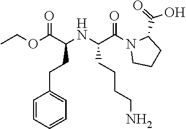 [SMILES: CCOC(=O)[C@H](CCc1ccccc1)N[C@@H](CCCCN)C(=O)N1CCC[C@H]1C(=O)O] | 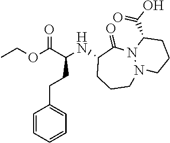 [SMILES: CCOC(=O)[C@H](CCc1ccccc1)N[C@H]1CCCN2CCC[C@@H](C(=O)O)N2C1=O] | 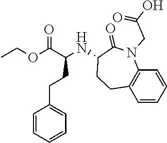 [SMILES: CCOC(=O)[C@H](CCc1ccccc1)N[C@H]1CCc2ccccc2N(CC(=O)O)C1=O] | 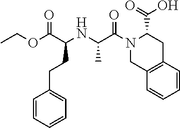 [SMILES: CCOC(=O)[C@H](CCc1ccccc1)N[C@@H](C)C(=O)N1Cc2ccccc2C[C@H]1C(=O)O] |
| Type | Non-prodrug | Prodrug | Prodrug | Prodrug |
| Molecular | 405.49 | 417.51 | 424.49 | 438.52 |
| Weight | | | | |
| Log P | −1.01 | 0.8 | 3.3 | 3.2 |
| pKa | 3.2, 10.2 | 3.4, 6.5 | 3.5, 5.4 | 3.7, 5.2 |
| ACE IC50 | 3.3 (lisinopril vs | 4 nM (cilazapril) | 425 nM (benazepril | 8.3 nM (Quinapril vs |
| | rabbit lung | 1.93 nM (cilizaprilat | vs rabbit lung ACE). | guinea pig serum |
| | ACE). | vs rabbit lung ACE). | 2 nM (benazeprilat | ACE). |
| | 5.6 (lisinopril vs | | vs rabbit lung ACE). | 2.8 nM (Quinaprilat |
| | serum ACE). | | Be | vs guinea pig serum |
| | | | | ACE). |
| Solubility | 97 mg/mL | 5 mg/mL | 1.76 mg/L (pH 7) | — |
| Half life | 12.6 hours | 9 hours (cilazaprilat) | 10-11 hours | 3 hours (quinaprilat) |
| | Long acting | Long acting | (benazeprilat) | Short acting |
| | | | Long acting | |

Beta Blockers

In certain embodiments of the invention, at least one beta-blocker or a pharmaceutically acceptable salt thereof is administered to a subject. A beta-blocker is any agent, natural or artificial, that blocks, inhibitors or at least reduces the binding of an agonist to a beta-adrenergic receptor and includes prodrugs. The beta-adrenergic receptor may be of any type including beta-1, beta-2, beta-3, or others (including selective or non-selective beta-blockers). Unless the context requires otherwise, reference to a beta-blocker herein should be taken to include reference to any pharmaceutically acceptable salt thereof. For the avoidance of doubt, reference to a beta-blocker herein is intended to include reference to stereoisomers thereof. A mixture of two or more beta-blockers may be used in methods of the invention.

Skilled persons will readily appreciate beta-blockers of use in the invention. However, by way of example, those described in Goodman and Gilman's the pharmacological basis of therapeutics, eleventh edition, chapter 10, pp 271-295, 2006, could be used. By way of further example, where a beta-blocker is used, it may be chosen for example from the group comprising alprenolol, bucindolol, carteolol, carvedilol, labetalol, levobunolol, medroxalol, mepindolol, metipranolol, nadolol, oxprenolol, penbutolol, pindolol, propafenone (a sodium channel blocking drug that also is a beta-adrenergic receptor antagonist), propranolol, sotalol, timolol or pharmaceutically acceptable salts thereof. By way of further example, where a beta blocker is used according to the present invention, it may be selected for example from the group comprising acebutolol, atenolol, betaxolol, bisoprolol, celiprolol, esmolol, metoprolol, nebivolol, or pharmaceutically acceptable salts thereof. In certain preferred embodiments, the beta-blocker used in the present invention is a non-selective beta-blocker. Skilled persons will readily appreciate non-selective beta-blockers, particularly having regard to the description and examples elsewhere herein. However, by way of example, non-selective beta-blockers include: propranolol, timolol, sotalol, pindolol, nadolol, and isomers thereof.

In certain preferred embodiments, a beta-blocker used in the present invention is chosen from the group comprising propranolol, timolol, atenolol, betaxolol, and/or nadolol and/or a or a pharmaceutically acceptable salt of any one thereof. In particular embodiments, a beta-blocker is chosen from propranolol, timolol, and/or atenolol and/or a pharmaceutically acceptable salt of any one thereof. In one particular embodiments, the beta-blocker is propranolol. By way of further example, the beta-blocker(s) may be chosen from (RS)-propranolol, (R)-propranolol, (S)-propranolol, (RS)-timolol, (S)-timolol, (R)-timolol, (RS)-atenolol, (S)-atenolol, (R)-atenolol.

In one embodiment, a beta-blocker is in a racemic form (ie a combination of enantiomers). In one embodiment, a beta-blocker comprises a combination of enantiomers, preferably comprising a higher proportion of the (R)-enantiomer. In one embodiment, a beta-blocker is substantially in the R-enantiomer form. In another embodiment, a beta-blocker is substantially in the S-enantiomer form.

ATIIR2 Antagonists

In certain embodiments of the invention, at least one ATIIR2 antagonist is administered to a subject. An ATIIR2 antagonist is any agent, natural or artificial, that blocks, inhibits or at least reduces the binding of an agonist to an angiotensin II type 2 receptor and includes prodrug forms of an ATIIR2 antagonist. Unless the context requires otherwise, reference to an ATIIR2 antagonist herein should be taken to include reference to any pharmaceutically acceptable salt thereof. For the avoidance of doubt, reference to an ATIIR2 antagonist herein is intended to include reference to stereoisomers thereof. A mixture of two or more ATIIR2 antagonists may be used in methods of the invention.

In certain embodiments, an ATIIR2 antagonist may be chosen from the groups described WO1993023378, WO1995003055, U.S. Pat. No. 5,173,493, WO2006066361, WO2011088504, WO2012010843, WO2013110135, WO2016142867, as incorporated by reference. In one embodiment, the ATIIR2 antagonist is chosen from the group comprising:
Olodanrigan (also known as EMA401, PD126055 or (S)-5-(benzyloxy)-2-(2,2-diphenylacetyl)-6-methoxy-1,2,3,4-tetrahydroisoquinoline-3-carboxylic acid);
PD123177 (also known as (S)-1-[(4-Amino-3-methylphenyl)methyl]-5-(diphenylacetyl)-4,5,6,7-tetrahydro-1H-Imidazo[4,5-c]pyridine-6-carboxylic acid trifluoroacetate salt);
SMM02 (also known as L-159686 (or L-159,686) or (S)-1,4-bis(N,N-diphenylcarbamoyl)piperazine-2-carboxylic acid);
PD123319 (also known as (S)-1-(4-(dimethylamino)-3-methylbenzyl)-5-(2,2-diphenylacetyl)-4,5,6,7-tetrahydro-1H-imidazo[4,5-c]pyridine-6-carboxylic acid);
EXP801 (also known as 2-[(N,N-Diphenylamino)carbonyl]-5-[(4-methoxyphenyl)methyl]-1,2,3,4-tetrahydroisoquinoline-3-carboxylic acid); and,
L161638 (also known as 2-Ethyl-6-[N-benzyl-N-(2-thienoyl)amino-3-[[2′-(1H-tetrazol-5-yl)-[1,1′]-biphenyl-4-yl]methyl]quinazolin-4-(3H)-one).

In one particular embodiment, the ATIIR2 antagonist(s) is chosen from the group comprising Olodanrigan (EMA401) and SMM02. SMM02 is a selective angiotensin II type 2 receptor (ATIIR2) inhibitor, and as mentioned herein before may also be known as L-159686 or L-159,686, for example. The different names for SMM02 may be used interchangeably herein.

Combinations of Different Compounds

In particular embodiments, the method comprises administering a combination of an ACEi and a beta-blocker. In certain embodiments, the ACEi is in prodrug form. In certain embodiments, the beta-blocker is a non-selective beta-blocker. In one embodiment, the method comprises administering a combination of an ACEi and a beta-blocker, wherein the ACEi is in prodrug form and the beta-blocker is a non-selective beta-blocker. In one embodiment, the ACEi is combined with a beta-blocker, where the beta-blocker is in a racemic form. In another embodiment, the ACEi is combined with a beta-blocker, where the beta-blocker is a combination of enantiomers, preferably comprising a higher proportion of the (R)-enantiomer. In another embodiment, the ACEi is used with a beta-blocker, where the beta-blocker is substantially in the (R)-enantiomer form. In a preferred embodiment, the ACEi is used with a beta-blocker, where the beta-blocker is substantially in the (R)-enantiomer form. In certain embodiments, the method comprises administering an ACEi, selected from the group: cilazapril, trandolapril, ramipril, quinapril, lisinopril, benazepril, or captopril, and a beta-blocker selected from the group: (RS)-propranolol, (R)-propranolol, (S)-propranolol, (RS)-timolol, (R)-timolol, (S)-timolol, (RS)-atenolol, (R)-atenolol, and (S)-atenolol. In certain embodiments, the method comprises administering a combination of cilazapril and (S)-propranolol, cilazapril and (S/R)-propranolol or, in one particular embodiment, cilazapril and (R)-propranolol. In other embodiments, the method comprises administering cilazapril and R-timolol, or cilazapril and S-timolol.

In particular embodiments, the method comprises administering a combination of: lisinopril and propranolol; lisinopril and timolol; lisinopril and atenolol; ramipril and propranolol; ramipril and timolol; ramipril and atenolol; trandolapril and timolol; trandolapril and propranolol; trandolapril and atenolol; cilazapril and propranolol; cilazapril and timolol; cilazapril and atenolol; benazepril and propranolol; benazepril and timolol; benazepril and atenolol; quinapril and propranolol; quinapril and timolol; quinapril and atenolol; enalapril and propranolol; enalapril and timolol; or, enalapril and atenolol.

In particular embodiments, the method comprises administering a combination of an ACEi and an ATIIR2 antagonist. In particular embodiments, the method comprises administering: cilazapril and EMA401; cilazapril and L-159686 (SMM02); Lisinopril and EMA401; Lisinopril and L-159686 (SMM02); ramipril and EMA401; ramipril and L-159686 (SMM02); trandolapril and EMA401; trandolapril and L-159686 (SMM02); benazepril and EMA401; benazepril and L-159686 (SMM02); quinapril and EMA401; quinapril and L-159686 (SMM02); enalapril and EMA401; or, enalapril and L-159686 (SMM02). In one embodiment, the method comprises administering cilazapril and EMA401. In another embodiment, the method comprises administering cilazapril and SMM02.

In particular embodiments, the method comprises administering a combination of an ATIIR2 antagonist and a beta-blocker, for example: EMA401 and propranolol; EMA401 and timolol; EMA401 and atenolol; L-159686 (SMM02) and propranolol; L-159686 (SMM02) and timolol; L-159686 (SMM02) and atenolol. In one embodiment, where an ATIIR2 antagonist and a beta-blocker are administered, the beta-blocker is in a racemic form. In another embodiment, the beta-blocker is a combination of enantiomers comprising a higher proportion of the (R)-enantiomer. In another embodiment, the beta-blocker is substantially in the (R)-enantiomer form. In one embodiment, the beta-blocker is a non-selective beta-blocker. In particular embodiments, the method comprises administering (R/S)-propranolol and SMM02, (S)-propranolol and SMM02 or, in one particular embodiment, (R)-propranolol and SMM02. In another embodiment, the method comprises administering (R/S)-propranolol and EMA401, (S)-propranolol and EMA401, or in one particular embodiment, (R)-propranolol and EMA401.

In particular embodiments, the method comprises administering a combination of an ACEi, a beta-blocker and an ATIIR2 antagonist. In certain embodiments, the method comprises administering: Lisinopril, propranolol, and EMA401; Lisinopril, timolol, and EMA401; Lisinopril, atenolol, and EMA401; Lisinopril, propranolol, and L-159686 (SMM02); Lisinopril, timolol, and L-159686 (SMM02); Lisinopril, atenolol, and L-159686 (SMM02); ramipril, propranolol, and EMA401; ramipril, timolol, and EMA401; ramipril, atenolol, and EMA401; ramipril, propranolol, and L-159686 (SMM02); ramipril, timolol, and L-159686 (SMM02); ramipril, atenolol, and L-159686 (SMM02); trandolapril, propranolol, and EMA401; trandolapril, timolol, and EMA401; trandolapril, atenolol, and EMA401; trandolapril, propranolol, and L-159686 (SMM02); trandolapril, timolol, and L-159686 (SMM02); trandolapril, atenolol, and L-159686 (SMM02); cilazapril, propranolol, and EMA401; cilazapril, timolol, and EMA401; cilazapril, atenolol, and EMA401; cilazapril, propranolol, and L-159686 (SMM02); cilazapril, timolol, and L-159686 (SMM02); cilazapril, atenolol, and L-159686 (SMM02); benazepril, propranolol, and EMA401; benazepril, timolol, and EMA401; benazepril, atenolol, and EMA401; benazepril, propranolol, and L-159686 (SMM02); benazepril, timolol, and L-159686 (SMM02); benazepril, atenolol, and L-159686 (SMM02); quinapril, propranolol, and EMA401; quinapril, timolol, and EMA401; quinapril, atenolol, and EMA401; quinapril, propranolol, and L-159686 (SMM02); quinapril, timolol, and L-159686 (SMM02); quinapril, atenolol, and L-159686 (SMM02); captopril, propranolol, and EMA401; captopril, timolol, and EMA401; captopril, atenolol, and EMA401; captopril, propranolol, and L-159686 (SMM02); captopril, timolol, and L-159686 (SMM02); captopril, atenolol, and L-159686L; enalapril, propranolol, and EMA401; enalapril, timolol, and EMA401; enalapril, atenolol, and EMA401; enalapril, propranolol, and L-159686 (SMM02); enalapril, timolol, and L-159686 (SMM02); or, enalapril, atenolol, and L-159686 (SMM02). The beta-blockers may be used in any isomeric form including a racemic mixture. In one embodiment, a beta-blocker comprises a combination of enantiomers, preferably comprising a higher proportion of the (R)-enantiomer. In another embodiment, the beta-blocker is substantially in the (S)-enantiomer form. In a preferred embodiment, the beta-blocker is substantially in the (R)-enantiomer form. In one embodiment, the beta-blocker is chosen from the group of non-selective beta-blockers.

In other embodiments, the methods comprise administering one or more of the specific combinations of agents as described and exemplified elsewhere herein.

In certain embodiments, the methods comprise local administration of: Cilazapril and R-propranolol; a) Ramipril or enalapril or trandalopril and b) R-propranolol; a) Cilazapril and b) S-propranolol or racemic (R/S) propranolol; a) Ramipril or enalapril or trandalopril and b) S-propranolol or racemic (R/S) propranolol; Cilazapril and timolol; a) Ramipril or enalapril or trandalopril and b) timolol; a) SMM02 or EMA401 and b) R-propranolol; a) SMM02 or EMA401 and b) S-propranolol or racemic (R/S) propranolol; a) SMM02 or EMA401 and b) timolol; a) SMM02 or EMA401 and b) cilazapril; or, a) SMM02 or EMA401 and b) Ramipril or enalapril or trandalopril.

In certain embodiments, the methods comprise systemically administering: Cilazapril and R-propranolol; Enalapril and R-propranolol; a) Ramipril or enalapril or trandalopril and b) R-propranolol; a) Cilazapril or ramipril or enalapril or trandalopril and b) R-timolol; a) Cilazapril or enalapril and b) racemic (R/S) propranolol or S-propranolol; SMM02 and R-propranolol; or, SMM02 and racemic (R/S) propranolol.

In certain embodiments, the methods comprise: locally administering Cilazapril and systemically administering propranolol (racemic (R/S) or R-); locally administering Ramipril or enalapril or trandalopril and systemically administering propranolol (racemic (R/S) or R-); locally administering R-propranolol and systemically administering Cilazapril or captopril or enalapril; locally administering timolol and systemically administering Cilazapril or captopril or enalapril; locally administering SMM02 or EMA401 and systemically administering propranolol (racemic (R/S) or R-); locally administering SMM02 or EMA401 and systemically administering cilazapril or captopril or enalapril; systemically administering SMM02 or EMA401 and locally administering propranolol (racemic (R/S) or R-) or timolol; or, systemically administering SMM02 or EMA401 and locally administering cilazapril or captopril or enalapril.

Dosage Forms and Compositions

Local

As mentioned previously herein, in certain aspects the invention provides methods for the treatment of hemangiomas by local delivery of: an ACEi; an ATIIR2 antagonist; an ACEi in combination with a beta-blocker; an ACEi in combination with an ATIIR2 antagonist; a combination of a beta-blocker and an ATIIR2 antagonist; or, a combination of an ACEi, beta-blocker and an ATIIR2 antagonist. In one embodiment, these agents are preferably formulated into compositions suitable for local administration.

Compositions of use in the invention will comprise at least one active agent (chosen from the group consisting ACEi, beta-blockers and ATIIR2 antagonists) and in a preferred embodiment one or more pharmaceutically acceptable carrier, diluent and/or excipient. In one embodiment, a composition includes a single type of active agent (for example, an ACEi, a beta-blocker or an ATIIR2 antagonist). In another embodiment, a composition includes a combination of two or more different types of active agents. By way of example, in one embodiment a composition of use in the invention comprises an ACEi. In another embodiment, a composition of use in the invention comprises a beta-blocker. In another embodiment, a composition of use in the invention comprises an ATIIR2 antagonist. In another embodiment, a composition comprises a combination of an ACEi and a beta-blocker. In another embodiment, a composition comprises a combination of an ACEi and an ATIIR2 antagonist. In another embodiment, a composition comprises a combination of a beta-blocker and an ATIIR2 antagonist. In another embodiment, a composition comprises a combination of an ACEi, a beta-blocker and an ATIIR2 antagonist.

Any of the ACEi, ATIIR2 antagonists and beta-blockers referred to herein may be formulated into compositions for local administration. However, in one embodiment, an ACEi is chosen from the group of ACEi in prodrug form, for example Enalapril, Ramipril, Trandolapril, Cilazapril, Benazepril, Perindopril, Imidapril, Fosinopril, Zofenopriland Quinapril and/or a pharmaceutically acceptable salt of any one thereof. In another preferred embodiment, an ACEi is chosen from the group consisting of ramipril, trandolapril, cilazapril and quinapril and/or a pharmaceutically acceptable salt of any one thereof. In one particular embodiment, the composition is formulated for topical administration and an ACEi is chosen from the group of ACEi in prodrug form, for example Enalapril, Ramipril, Trandolapril, Cilazapril, Benazepril and Quinapril and/or a pharmaceutically acceptable salt of any one thereof. In another preferred embodiment, the composition is formulated for topical administration and an ACEi is chosen from the group consisting of ramipril, trandolapril, cilazapril and quinapril. In another preferred embodiment, where the composition is formulated for topical administration, a beta-blocker is chosen from the group comprising timolol, propranolol, atenolol, betaxolol, bisoprolol, nadolol, carvedilol, carazolol and/or a pharmaceutically acceptable salt of any one thereof. The beta-blockers may be used in any isomeric form including a racemic mixture. In one embodiment, a beta-blocker comprises a combination of enantiomers, preferably comprising a higher proportion of the (R)-enantiomer. In one embodiment, the beta-blocker(s) is substantially in the (S)-enantiomer form. In a preferred embodiment, the beta-blocker(s) is substantially in the (R)-enantiomer form. In one embodiment, the beta-blocker is R-propranolol. In one embodiment, the beta-blockers are chosen from the group comprising non-selective beta-blockers. In another preferred embodiment, where the composition is formulated for topical administration, an ATIIR2 antagonist is chosen from the group comprising olodanrigan (EMA401), PD123177, L-159686 (SMM02), PD123319, PD126055, EXP801, and L161638 and/or a pharmaceutically acceptable salt of any one thereof.

In one embodiment, a composition of the invention comprises an ACEi in the form of a prodrug and a non-selective beta-blocker.

In certain embodiments, a composition of the invention comprises: lisinopril and propranolol; lisinopril and timolol; lisinopril and atenolol; ramipril and propranolol; ramipril and timolol; ramipril and atenolol; trandolapril and timolol; trandolapril and propranolol; trandolapril and atenolol; cilazapril and propranolol; cilazapril and timolol; cilazapril and atenolol; benazepril and propranolol; benazepril and timolol; benazepril and atenolol; quinapril and propranolol; quinapril and timolol; quinapril and atenolol; enalapril and propranolol; enalapril and timolol; or, enalapril and atenolol. In one embodiment, the beta-blockers used are non-selective beta-blockers. The beta-blockers may be used in any isomeric form including a racemic mixture. In one embodiment, a beta-blocker comprises a combination of enantiomers, preferably comprising a higher proportion of the (R)-enantiomer. In one embodiment, the beta-blocker(s) is substantially in the (R)-enantiomer form. In one embodiment, the beta-blocker(s) is substantially in the (S)-enantiomer form. In a preferred embodiment, the beta-blocker is R-propranolol. In certain embodiments, a composition of the invention comprises: R-timolol and cilazapril; S-timolol and cilazapril; R/S-timolol and cilazapril; R-propranolol and cilazapril; S-propranolol and cilazapril; R/S-propranolol and cilazapril; SMM02 and cilazapril; EMA401 and cilazapril; R-propranolol and EMA401; S-propranolol and EMA401; R/S-propranolol and EMA401; R-propranolol and SMM02; S-propranolol and SMM02; R/S-propranolol and SMM02; EMA401 and cilazapril; SMM02 and S-timolol; SMM02 and R-timolol; EMA401 and S-timolol; or, EMA401 and R-timolol.

In other embodiments, a composition of the invention comprises one of the specific combinations of agents described and exemplified herein as being administered or of use in the methods of the invention.

Skilled persons will readily appreciate types of compositions suitable for local administration including injectable compositions and topical compositions, such as gels, suspensions, oils, emulsions, sprays, powders, creams, foams, lotions, ointments, drops and the like. In one embodiment, the composition is suitable for ocular administration (for example, eye drops or other suitable ophthalmic compositions). In preferred embodiments, the compositions are formulated as emulsion gels, liposome gels, liposome emulsion gels or proliposome emulsion gels. In another embodiment, the invention provides a drug delivery device suitable for local administration and comprising one or more of the active agents (including reference to combinations of active agents) or compositions as described herein. In one embodiment, the drug delivery device comprises one of the specific combinations of active agents described and exemplified herein as being administered or of use in the methods of the invention. Skilled persons will readily appreciate appropriate drug delivery devices for local administration and means for manufacturing them. However, by way of example, in one embodiment the drug delivery device is a dermal patch suitable for local administration.

Skilled persons will readily appreciate a variety of suitable, carriers, excipients and diluents of use in formulating compositions for local delivery, including injectable, compositions and topical compositions, having regard to the nature of the invention described herein and the information published in pharmaceutical texts and handbooks, such as The Handbook of Pharmaceutical Excipients, Sixth edition, 2009, editors Raymond C Rose, Paul J Sheskey and Marian E Quinn (http://pharmama.info/wp-content/uploads/2018/10/Excipients.pdf).

In a preferred embodiment, compositions for topical administration include a skin penetration enhancer. A “skin penetration enhancer” as referred to herein is any chemical, agent, or vehicle which can increase improve rate of delivery of an agent through the skin and/or increase the depth of penetration of the agent into a hemangioma. Examples of penetration enhancers include, but are not limited to: mineral oils; dimethyl sulfoxide; glycols, for example propylene glycol, 1,3-butylene glycol, diethylene glycol monoethyl ether, and polyethylene glycols of any molecular weight; alcohols, for example ethanol and isopropyl alcohol; surfactants and emulsifiers, for example oleyl oleate, sorbitan monooleate, octyl dodecanol, isopropyl palmitate, oleyl alcohol, ethyl oleate, glycerol monolaurate, isopropyl myristate, lauryl lactate, span 20-span 80, tween 20-tween 80, N-laurylsarcosine, oleic acid, sodium octyl sulfate, methyl laurate, sodium lauryl sulfoacetate, coco-caprylate, cetyl alcohol, Transcutols including Transcutol P, Azone, stearyl alcohol, or combinations thereof; phospholipids, for example lecithin, soy phosphatidylcholine, hydrogenated soy phosphatidylcholine, DOPC, DOPE, DPPC, DPPE, DSPC, DSPE, DOPG, DPPG, DSPG; terpenes, for example menthol, limonene, linalool, cineole, nerolidol, farnesol, geraniol, carvone, terpinolene, ascaridole, and other essential oils; or any other agent which can increase the rate of permeation of drugs through skin. In addition, carrier systems or vehicles such as liposomes and proliposomes may be used to enhance topical delivery.

Compositions of use in the methods of the invention may optionally include one or more additional ingredients which may be of benefit to include in such compositions, such as other agents which may be of benefit to the subject. For example, they may include one or more additional active agent which is of benefit in treating hemangioma or otherwise of benefit to the health or cosmetic appearance of a subject.

The compositions may include any appropriate amount of an active agent (an ACEi, a beta-blocker and/or an ATIIR2 antagonist) having regard to the desired dose to be delivered to a subject (as described elsewhere herein) and the agent's chemical properties as will be understood by persons skilled in the art having regard to the description herein. However, by way of example, for a composition for local administration (for example, topical) the total amount of active agents (ACEi, beta-blocker and/or ATIIR2) present in the composition may be from approximately 0.01% to approximately 20% (w/w). In particular embodiments, the following ranges may be used for a topical composition: approximately 0.01% to approximately 19.99% of ACEi, preferably approximately 0.1% to approximately 10% or to approximately 8%; approximately 0.01% to approximately 19.99% of beta-blocker, preferably approximately 0.1% to approximately 10% or to approximately 8%; and, approximately 0.01% to approximately 19.99% of ATIIR2 antagonist, preferably approximately 0.1% to approximately 5%.

In embodiments of the invention where two or more different agents are combined in a single composition (for example, ACEi and beta-blocker, ACEi and ATIIR2, ATIIR2 and beta-blocker or ACEi, ATIIR2 and beta-blocker) they may be combined in any appropriate ratios. For example: an ACEi and beta-blocker may be combined in a ratio from approximately 10:1 to 1:10; an ACEi and an ATIIR2 antagonist may be combined in a ratio from approximately 10:1 to 1:10; or, a beta-blocker and an ATIIR2 antagonist may be combined in a ratio from approximately 10:1 to 1:10.

In one particular embodiment, topical fixed-dose combinations of ACE inhibitors and beta-blockers include a greater proportion of ACE inhibitor relative to beta blocker, as the inventors have surprisingly identified that beta-blockers absorb more rapidly than ACE inhibitors through the skin. In one embodiment, the ratio of ACEi to beta-blocker in a topical composition is from approximately 1:1 to approximately 10:1. In other embodiments, the ratio is from approximately 2:1 to approximately 10:1. In other embodiments, the ratio of ACEi to beta-blocker in the composition is from approximately 3:1 to approximately 9:1, approximately 4:1 to approximately 8:1, or approximately 3:1 to approximately 7:1. In other embodiments the ratio is approximately 1:1, approximately 2:1, approximately 3:1, approximately 4:1, approximately 5:1, approximately 6:1, approximately 7:1, approximately 8:1, approximately 9:1 or approximately 10:1. In other embodiments, there may be a greater proportion of beta-blocker relative to ACE inhibitor in the composition.

In a particular embodiment, the ratio of beta-blocker to ATIIR2 antagonist in a composition is from approximately 1:2 to approximately 1:10, for example, approximately 1:2, approximately 1:3, approximately 1:4, approximately 1:5, approximately 1:6, approximately 1:7, approximately 1:8, approximately 1:9, or approximately 1:10.

In a particular embodiment, the ratio of beta-blocker to ACEi in a composition is from approximately 1:2 to approximately 1:9, for example, approximately 1:2, approximately 1:3, approximately 1:4, approximately 1:5, approximately 1:6, approximately 1:7, approximately 1:8, or approximately 1:9.

In a particular embodiment, the ratio of ATIIR2 antagonist to ACEi in a composition is from approximately 1:1 to approximately 1:5, for example, approximately 1:1, approximately 1:2, approximately 1:3, approximately 1:4, or approximately 1:5.

In certain embodiments, the ratio of beta-blocker to ATIIR2 antagonist in a composition is approximately 1:2.5, approximately 1:5, or approximately 1:10.

In certain embodiments, the ratio of beta-blocker to ACEi in a composition is approximately 1:2, approximately 1:4 or approximately 1:9.

In certain embodiments, the ratio of ATIIR2 antagonist to ACEi in a composition is approximately 1:2, approximately 1:1, or approximately 1:4.

In particular embodiments, compositions for local administration include: cilazapril and timolol in a ratio of approximately 1:1, approximately 1:10 or approximately 10:1; timolol and EMA401 in a ratio of approximately 1:10 or approximately 1:1; cilazapril and EMA401 in a ratio of approximately 1:10 or approximately 1:1; ramipril and propranolol in a ratio of approximately 1:1; ramipril and propranolol in a ratio of from approximately 1:1; or trandolapril and timolol in a ratio of approximately 4:1. In other embodiments, the compositions comprising a combination of two or more agents for local administration include the combinations of compounds approximately at the ratios as specified in the examples and Table 6 herein after.

Compositions for local administration may be made in accordance with standard techniques as may be found in such standard references as Gennaro A R: Remington: The Science and Practice of Pharmacy, 22nd ed., Lippincott, Williams & Wilkins, for example. In preferred embodiments, the formulations and methods described and exemplified herein may be used.

In one particular embodiment the active agents of use in the invention are administered topically in an emulsion gel. In one embodiment, the composition comprises an ACE inhibitor selected from the group described hereinbefore (for example lisinopril, Enalapril, Ramipril, Trandolapril, Cilazapril, Benazepril, perindopril, imidapril, fosinopril, zofenopril, captopril and/or Quinapril), alone or in combination with a β-blocker selected from the group described herein before (for example propranolol, timolol, atenolol, betaxolol, nadolol and are prepared as an emulsion gel for topical application. The concentration of ACE inhibitor and β-blocker may range from approximately 0.01 to approximately 20 weight percentage (wt. %). Preferably, the concentrations of ACE inhibitor and/or β-blocker may range from approximately 0.1 wt. % (or from approximately 0.5 wt %) to approximately 5 wt %, and more preferably from approximately 0.2 wt % (or from approximately 0.5 wt %) to approximately 2 wt %. The gel may be selected from a carbomer, a carboxymethyl cellulose, a hydroxypropyl carboxymethyl cellulose, a polyvinylpyrridone, a polyvinylacetate, a chitosan, a succinylated chitosan, a dextran, an alganic acid, a xanthan gum, or any other gelling agent suitable for pharmaceutical preparation. Preferably, the gel may be a carbomer, or more preferably, selected from either carbopol ultrez 10 or carbopol ultrez 30. Furthermore, the formulation may contain skin permeation enhancers selected from the group described hereinbefore (for example, a combination of isopropyl alcohol, propylene glycol, coco-caprylate, mineral oil, and Tween 80) in a ratio sufficient to maximise the permeation of the active ingredients. The formulation may additionally contain agents to increase the solubility of the ACE inhibitor and/or β-blocker (for example cyclodextrins, particularly 2-hydroxypropyl-β-cyclodextrin) and may contain an alkali agent (for example sodium hydroxide, potassium hydroxide, or ammonia) to neutralise the mixture to a final acidity of pH of 6-8. To increase the shelf-life of the pharmaceutical preparation, preservative agents, (for example, parabens, in preferred embodiments methylparaben and/or propylparaben) may be included.

In another particular embodiment, the active agents may be administered topically in the form of a liposome gel. In one embodiment, the liposomes prepared for use in the liposome gel composition are preferably deformable liposomes, ultradeformable liposomes, or transfersomes. Phospholipid material employed for the preparation of the liposomes may be selected from any appropriate phospholipids as would be understood by persons skilled in the art. However, by way of example, they may be chosen from the group comprising: synthetic vesicle-forming lipids and naturally-occurring vesicle-forming lipids, including for example, phosphatidylcholines (PC) such as soybean phosphatidylcholine (SPC), 1,2-dioleoyl-sn-glycero-3-phosphocholine (DOPC), 1,2-dipalmitoyl-sn-glycero-3-phosphocholine (DPPC), 1,2-distearoyl-sn-glycero-2-phosphatidylcholine (DSPC), 1-myristoyl-2-stearoyl-sn-glycero-3-phosphocholine (MSPC), hydrogenated soybean phosphatidylcholine (HSPC); phosphatidylinositol (PI), phosphatidylglycerol (PG), dimyristoyl phosphatidylglycerol (DMPG); egg yolk phosphatidylcholine (EPC), 1-palmitoyl-2-oleoylphosphatidyl choline (POPC), distearoylphosphatidylcholine (DSPC), dimyristoyl phosphatidylcholine (DMPC); phosphatidic acid (PA), phosphatidylserine (PS); 1-palmitoyl-2-oleoylphosphatidyl choline (POPC), and the sphingophospholipids such as sphingomyelins (SM) having 12- to 24-carbon atom acyl or alkyl chains. In a preferred embodiment, the phospholipids have a low phase transition temperature (below approximately 37° C.), as higher phase transition temperature phospholipids tend to form ridged membranes which resist deformation. For example, the phospholipid selected may preferably include soybean lecithin, soybean phosphatidylcholine, egg yolk lecithin, egg yolk phosphatidylcholine. The phospholipid material may be used in combination with a surfactant or edge activator to impart structural flexibility or deformability to the liposome vesicle. An Edge activator” is a surfactant or chemical agent which is capable of increasing the elasticity of the phospholipid liposome membrane, and may include, for example, polysorbates including the Tween and Span families, sorbitan monooleate, bile salts, and alcohols including ethanol and isopropyl alcohol. Preferably, the edge activator used in the formulation is a polysorbate, for example Tween 80. A liposome suspension may be prepared by any number of liposome preparation methods, but preferably may include the thin-film rehydration method, where a film of the phospholipid/edge activator mixture is prepared. The liposomes can be passively or actively loaded by subsequently suspending the phospholipid mixture in an aqueous solution of the active pharmaceutical agent(s) or active loading agent, for example ammonium sulfate, ammonium oxalate, ammonium phosphate, ammonium sucrose octasulfate, or calcium acetate. The liposome suspension can then undergo ultrasonication or extrusion to form uniform liposomes with controlled size in the range of about 10-1000 nm, or preferably in the range of about 50-200 nm. In the case of an active loading method, the liposome suspension with uniform size is then subjected to a method, such as dialysis or chromatography, to create a gradient or difference in concentration of the selected active loading agent between the liposome interior and external medium, which can be used to load the desired active pharmaceutical agent upon mixing a solution of the drug with the liposome suspension. The liposome suspension may be used alone or combined with a gel or thickening agent selected from the group herein before described (for example a carbomer) for therapeutic application. An alkali agent, for example sodium hydroxide or ammonia may be used to neutralise the final preparation to an acidity of pH 6-8, and preservatives, for example parabens may be included in the manufacture.

In a further embodiment, the active agents may be administered topically using a liposome emulsion gel, or proliposome gel, as a carrier formulation. The appropriate combination of phospholipids and edge activators (for example soybean phosphatidylcholine and polysorbate 80), may be dissolved in a solvent (for example ethanol, isopropyl alcohol, propylene glycol, or combination thereof). In certain cases, the solvent may additionally act as a skin penetration enhancer, for example, when selected from the group described hereinbefore (for example propylene glycol, ethanol and isopropyl alcohol). In one embodiment, the solvent preferably comprises equal amounts of propylene glycol and isopropyl alcohol. The phospholipid solution can then be combined with the appropriate portion of an aqueous solution containing the active agent(s) at a concentration sufficient to give a final concentration in the preparation of from approximately 0.1% (or from approximately 0.01%) to approximately 20%, or from approximately 0.1% to approximately 10%, or from approximately 0.1% to approximately 8%, or preferably between approximately 0.5% and approximately 5%. The mixture can be combined with a gel or thickening agent, for example a carbomer. An alkali agent, for example sodium hydroxide or ammonia may be used to neutralise the final preparation to an acidity of pH 6-8, and preservatives, for example parabens, may be included in the manufacture.

In another particular embodiment, the active agent(s) may be administered topically in the form of a cream. The concentrations of the active pharmaceutical ingredient(s) may range from approximately 0.01% (or from approximately 0.1%) to approximately 20% (w/w), for example from approximately 0.1% to approximately 10% (or to approximately 8%), or from approximately 0.1 wt % (or from approximately 0.5%) to approximately 5 wt. %, and in one embodiment may be approximately 0.5% to approximately 2%. In this embodiment, the active agent(s) are formulated with: at least one fatty alcohol emulsifier, selected from the group comprising cetyl alcohol and stearyl alcohol; an oil-in-water emulsifying agent; at least one preservative; at least one penetration enhancer selected from the group consisting of diethylene glycol monoethyl ether and isopropyl myristate; and an alkali agent such as sodium hydroxide to neutralise the mixture to a final acidity of pH 6-8.

In particular embodiments, the active agent(s) may be administered topically in the form of: a cream or gel comprising approximately 0.1 to approximately 1% beta blocker selected from propranolol or timolol; a cream or gel comprising approximately 0.1 to approximately 1% ACE inhibitor selected the group comprising cilazapril, ramipril, trandolapril, enalapril, quinapril; a cream or gel comprising approximately 0.1 to approximately 1% ACE inhibitor selected from the group comprising cilazapril, ramipril, trandolapril, enalapril, quinapril, and approximately 0.1 to approximately 1% beta blocker selected from the group comprising propranolol and timolol; a cream or gel comprising approximately 0.1 to approximately 1% cilazapril and approximately 0.1 to approximately 1% propranolol; a cream or gel comprising approximately 0.1 to approximately 1% cilazapril and approximately 0.1 to approximately 1% timolol; a cream or gel comprising approximately 0.1 to approximately 1% ramipril and approximately 0.1 to approximately 1% propranolol; or, a cream or gel comprising approximately 0.1 to approximately 2% trandolapril and approximately 0.1 to approximately 0.5% timolol.

Systemic

In certain embodiments, the methods of the invention comprise administering a combination of two or more of a beta-blocker, an ACEi and an ATIIR2 antagonist systemically to a subject, or administration of one or more of these agents in addition to locally administering an ACEi, beta-blocker and/or ATIIR2. In one embodiment, these agents are preferably formulated into compositions suitable for systemic administration. Such compositions will comprise at least one active agent (chosen from the group consisting ACEi, beta-blockers and ATIIR2 antagonists) and in a preferred embodiment one or more pharmaceutically acceptable carrier, diluent and/or excipient. In one embodiment, the composition is suitable for oral administration. For paediatric applications, a liquid formulation is preferred. In another embodiment, the composition is suitable for administration by way of injection; for example, subcutaneous, intramuscular or intravenous injection.

Compositions suitable for systemic administration in accordance with the invention will comprise at least one active agent (chosen from the group consisting of ACEi, beta-blockers and ATIIR2 antagonists) and preferably one or more pharmaceutically acceptable carrier, diluent and/or excipient. In one embodiment, a systemic composition includes a single type of active agent (for example, an ACEi, a beta-blocker or an ATIIR2 antagonist). In another embodiment, a systemic composition includes a combination of two or more different types of active agents. By way of example, in one embodiment a composition of use in the invention comprises an ACEi. In another embodiment, a composition of use in the invention comprises a beta-blocker. In another embodiment, a composition of use in the invention comprises an ATIIR2 antagonist. In another embodiment, a composition comprises a combination of an ACEi and a beta-blocker. In another embodiment, a composition comprises a combination of an ACEi and an ATIIR2 antagonist. In another embodiment, a composition comprises a combination of a beta-blocker and an ATIIR2 antagonist. In another embodiment, a composition comprises a combination of an ACEi, a beta-blocker and an ATIIR2 antagonist.

Any of the ACEi, ATIIR2 antagonists and beta-blockers referred to herein may be formulated into compositions for systemic administration. However, in one embodiment, an ACEi is chosen from the group of Captopril, Enalapril, Ramipril, Trandolapril, Cilazapril, Benazepril, Perindopril, Imidapril, Fosinopril, Zofenopriland Quinapril and/or a pharmaceutically acceptable salt of any one thereof. In one embodiment, the ACEi is a prodrug. In one embodiment, the beta-blocker is chosen from the group of non-selective beta-blockers. In a preferred embodiment, a beta-blocker is chosen from the group comprising timolol, propranolol, atenolol, betaxolol, bisoprolol, nadolol, carvedilol, carazolol and/or a pharmaceutically acceptable salt of any one thereof. In one embodiment, the beta-blocker is in R- or S-form or is a racemic mix of both. In one embodiment, there is a racemic mix of a beta-blocker having a higher proportion of the R-enantiomer. In one embodiment, the beta-blocker is in substantially the R-enantiomeric form. In another embodiment, an ATIIR2 is chosen from the group comprising olodanrigan (EMA401), PD123177, L-159686, PD123319, PD126055, EXP801, and L161638 and/or a pharmaceutically acceptable salt of any one thereof.

In a preferred embodiment, the composition for systemic administration includes a beta-blocker (for example propranolol and/or timolol and/or atenolol) and/or an ACEi (such as captopril, cilazapril lisinopril, trandolapril, ramipril or enalapril) and/or an ATIIR2. In one embodiment, the composition comprises an ACEi in the form of a prodrug and a non-selective beta-blocker.

In certain embodiments, a systemic composition of use in the invention comprises: lisinopril and propranolol.; lisinopril and timolol; lisinopril and atenolol; ramipril and propranolol; ramipril and timolol; ramipril and atenolol; trandolapril and timolol; trandolapril and propranolol; trandolapril and atenolol; cilazapril and propranolol; cilazapril and timolol; cilazapril and atenolol; benazepril and propranolol; benazepril and timolol; benazepril and atenolol; quinapril and propranolol; quinapril and timolol; quinapril and atenolol; enalapril and propranolol; enalapril and timolol; or, enalapril and atenolol. In one embodiment, the beta-blocker is in substantially the R-enantiomeric form.

In other embodiments, a systemic composition of the invention comprises one of the specific combinations of agents described and exemplified herein as being administered or of use in the methods of the invention.

Systemic compositions of use in these embodiments of the methods of the invention may optionally include one or more additional ingredients which may be of benefit to include in such compositions, such as other agents which may be of benefit to the subject. For example, they may include one or more additional active agent which is of benefit in treating hemangioma or otherwise of benefit to the health or cosmetic appearance of a subject.

Skilled persons will readily appreciate types of compositions suitable for systemic administration including injectable compositions, pills, capsules, gels, suspensions, oils, emulsions, sprays, powders, liquids and the like. In one embodiment, the composition is suitable for oral administration. In preferred embodiments, the compositions are formulated oral liquid formulations. In another embodiment, the invention provides a drug delivery device suitable for systemic administration and comprising one or more of the active agents (including reference to combinations of active agents) or compositions as described herein. In one embodiment, the drug delivery device comprises one of the specific combinations of active agents described and exemplified herein as being administered or of use in the methods of the invention. Skilled persons will readily appreciate appropriate drug delivery devices for systemic administration and means for manufacturing them. In one embodiment, the drug delivery device is a transdermal patch suitable for systemic administration.

Skilled persons will readily appreciate a variety of suitable carriers, excipients and diluents of use in formulating compositions for systemic delivery, including injectable compositions and oral compositions, for example, having regard to the nature of the invention described herein and the information published in pharmaceutical texts and handbooks, such as The Handbook of Pharmaceutical Excipients, Sixth edition, 2009, editors Raymond C Rose, Paul J Sheskey and Marian E Quinn (http://pharmama.info/wp-content/uploads/2018/10/Excipients.pdf).

The compositions suitable for systemic administration may include any appropriate amount of an active agent (an ACEi, a beta-blocker and an ATIIR2 antagonist) having regard to the desired dose to be delivered to a subject (as described elsewhere herein) and the agent's chemical properties as will be understood by persons skilled in the art having regard to the description herein.

However, by way of example, for a composition for oral administration, or an injectable composition, the total amount of active agents (ACEi, beta-blocker and/or ATIIR2) present in the composition may be from approximately 0.001 mg to approximately 500 mg or approximately 1000 mg. In particular embodiments, the following ranges may be used for an oral composition: approximately 0.1 mg to approximately 100 mg of ACEi, preferably approximately 0.5 mg to approximately 20 mg; approximately 0.1 mg to approximately 150 mg of beta-blocker, preferably about approximately 0.5 mg to approximately 100 mg and, approximately 0.01 mg to approximately 400 mg of ATIIR2 antagonist, preferably approximately 0.5 mg to approximately 100 mg.

In embodiments of the invention where two or more different agents are combined in a single composition (for example, ACEi and beta-blocker, ACEi and ATIIR2, ATIIR2 and beta-blocker or ACEi, ATIIR2 and beta-blocker) they may be combined in any appropriate ratios. For example: an ACEi and beta-blocker may be combined in a ratio from approximately 10:1 to 1:10; an ACEi and an ATIIR2 antagonist may be combined in a ratio from approximately 10:1 to 1:10; or, a beta-blocker and an ATIIR2 antagonist may be combined in a ratio from approximately 10:1 to 1:10.

The ACEi, ATIIR2 antagonists and beta-blockers of use for systemic administration includes any of those exemplified elsewhere herein. Similarly, exemplary ratios of one compound to another (in a single composition or as used or administered in a method of the invention) provided elsewhere herein may be applied to systemic compositions and delivery methods.

Compositions suitable for systemic administration may be formulated in accordance with standard techniques as may be found in such standard references as Gennaro A R: Remington: The Science and Practice of Pharmacy, 22nded., Lippincott, Williams & Wilkins, for example.

Dosage and Administration Regimes

The dose of an active agent (ACEi, ATIIR2 antagonist, and/or beta-blocker) or composition administered, the period of administration, and the general administration regime may differ between subjects depending on such variables as the size and/or severity of the hemangioma to be treated, the type of hemangioma to be treated, the stage of development of the hemangioma, the type of agent or composition to be administered, the administration route, size of a unit dosage, the kind of excipients, carriers and the like used, the age and/or general health of a subject, and other factors well known to those of ordinary skill in the art. A skilled person will be able to determine the appropriate dose sufficient to deliver an effective amount of active agent(s) to a subject or hemangioma having regard to such factors and the information contained herein. By way of example, in the case of a severe or rapidly proliferating hemangioma a larger daily dose and/or a longer treatment period may be employed, compared to the treatment of a less severe hemangioma. In some cases, for example where the growth of a hemangioma is particularly severe, local treatment of a hemangioma in accordance with the invention can be augmented with systemic administration of one or more agent which may assist in the treatment of hemangioma, including an ACEi, beta-blocker and/or ATIIR2. In one particular embodiment, a beta-blocker and/or an ACEi is administered systemically in addition to local administration of an active agent or combination of active agents or a composition or compositions comprising same in accordance with the invention.

Administration may include a single daily dose or administration of a number of discrete divided doses as may be appropriate. An administration regime may also include administration of one or more of the active agents, or compositions comprising same, as described herein.

By way of example only, an ACEi, ATIIR2 antagonist and/or a beta-blocker, or compositions comprising one or a combination of these agents, may be administered (for example, applied (preferably topically)) between from 1 to 10 times per day, preferably between from 1 to 5 times per day or between from 1 to 3 times per day, more preferably once or twice a day. By way of further example, the treatment may occur for about 6 to 14 months and preferably for the duration of the proliferative phase of the hemangioma, as this may reduce the risk of rebound or regrowth of the lesion. Skilled persons will be able to readily recognise the proliferative phase the of the hemangioma from the age of the hemangioma and the growth rate of the lesion. In the case of infantile hemangioma a skilled person will readily recognise this phase from the age of the infant and the growth rate of the lesion. However, by way of example only, for infantile hemangioma, the proliferative phase typically occurs within the first 12 months of age. In many cases, infantile hemangiomas can reach 80% maximum size in the first three months.

In certain embodiments, where an active agent (ACEi, beta-blocker and/or ATIIR2 antagonist) is administered locally (preferably topically) it is preferably administered to provide a daily dose of active agent(s) from approximately 0.01 mg/cm² to approximately 50 or approximately 100 mg/cm² of an individual agent (measured with reference to the size of the hemangioma). In certain embodiments, where an active agent (ACEi, beta-blocker and/or ATIIR2 antagonist) is administered topically it is preferably administered in a sufficient quantity to form a thin layer of the composition over substantially the whole surface of the hemangioma lesion (including reference to the skin or other tissue surface at the site of a hemangioma, for example overlying the hemangioma). In one embodiment, where an active agent (ACEi, beta-blocker and/or ATIIR2 antagonist) is administered locally by intralesional injection (or injection to a tissue proximal to the hemangioma) it is preferably administered to provide a daily dose of active agent(s) from approximately 0.01 mg to approximately 50 or approximately 100 mg of an individual agent to a subject. In certain embodiments, where an active agent (ACEi, beta-blocker and/or ATIIR2 antagonist) is administered by intralesional injection (or injection to a tissue proximal to the hemangioma) it is preferably administered in a manner sufficient to distribute or administer the active agent(s) substantially evenly throughout the hemangioma.

In one particular embodiment, an ACEi is topically administered to a subject to provide a daily dose of from approximately 0.01 mg/cm² to approximately 100 mg/cm² (measured with reference to the size of the hemangioma).

In one particular embodiment, an ATIIR2 antagonist is topically administered to a subject to provide a daily dose of from approximately 0.01 mg/cm² to 100 mg/cm² (measured with reference to the size of the hemangioma).

In another embodiment, a beta-blocker and an ACEi are administered topically to a subject. In one embodiment, the beta-blocker and ACEi are administered topically to provide a daily dose of active agents from approximately 0.01 mg/cm² to approximately 100 mg/cm² (measured with reference to the size of the hemangioma). In one particular embodiment, a higher dose of ACEi is administered to a subject relative to the dose of beta-blocker administered.

In another embodiment, an ACEi and an ATIIR2 antagonist are administered topically to a subject. In one embodiment, the ACEi and ATIIR2 antagonist are administered topically to provide a daily dose of active agents from approximately 0.01 mg/cm² to approximately 100 mg/cm²⁻ (measured with reference to the size of the hemangioma).

In another embodiment, a beta-blocker and an ATIIR2 antagonist are administered topically to a subject. In one embodiment, the beta-blocker and ATIIR2 antagonist are administered topically to provide a daily dose of active agents from approximately 0.01 mg/cm² to approximately 100 mg/cm² (measured with reference to the size of the hemangioma).

In another embodiment, an ACEi, beta-blocker and an ATIIR2 antagonist are administered topically to a subject. In one embodiment, the ACEi, beta-blocker and ATIIR2 antagonist are administered topically to provide a daily dose of from approximately 0.01 mg/cm² to approximately 100 mg/cm² (measured with reference to the size of the hemangioma).

In embodiments of the invention: an ACEi and beta-blocker are administered locally in a ratio of from approximately 10:1 to 1:10; an ACEi and an ATIIR2 antagonist are administered locally in a ratio of from approximately 10:1 to 1:10; or, a beta-blocker and an ATIIR2 antagonist are administered locally in a ratio of from approximately 10:1 to 1:10.

In one particular embodiment, an ACEi and beta-blocker are administered locally, preferably topically to a subject, so that a greater proportion of ACE inhibitor relative to beta blocker is administered. In one embodiment, the ratio of ACEi to beta-blocker administered is from approximately 1:1 to approximately 10:1. In another embodiment the ratio is from approximately 2:1 to approximately 10:1. In other embodiments, the ratio of ACEi to beta-blocker in the composition is from approximately 3:1 to approximately 9:1, approximately 4:1 to approximately 8:1 or approximately 5:1 to approximately 7:1. In other embodiments the ratio is approximately 1:1, 2:1, approximately 3:1, approximately 4:1, approximately 5:1, approximately 6:1, approximately 7:1, approximately 8:1, approximately 9:1 or approximately 10:1. In other embodiments, a greater proportion of beta-blocker relative to ACE inhibitor may be administered.

In a particular embodiment, the ratio of beta-blocker to ATIIR2 antagonist administered is from approximately 1:2 to approximately 1:10, for example, approximately 1:2, approximately 1:3, approximately 1:4, approximately 1:5, approximately 1:6, approximately 1:7, approximately 1:8, approximately 1:9, or approximately 1:10.

In a particular embodiment, the ratio of beta-blocker to ACEi is administered from approximately 1:2 to approximately 1:9, for example, approximately 1:2, approximately 1:3, approximately 1:4, approximately 1:5, approximately 1:6, approximately 1:7, approximately 1:8, or approximately 1:9.

In a particular embodiment, the ratio of ATIIR2 antagonist to ACEi administered is from approximately 1:1 to approximately 1:5, for example, approximately 1:1, approximately 1:2, approximately 1:3, approximately 1:4, or approximately 1:5.

In certain embodiments, the ratio of beta-blocker to ATIIR2 antagonist administered is approximately 1:2.5, approximately 1:5, or approximately 1:10.

In certain embodiments, the ratio of beta-blocker to ACEi administered is approximately 1:2, approximately 1:4 or approximately 1:9.

In certain embodiments, the ratio of ATIIR2 antagonist to ACEi administered is approximately 1:2, approximately 1:1, or approximately 1:4.

In particular embodiments: cilazapril and timolol are administered locally in a ratio of approximately 1:1, approximately 1:10 or approximately 10:1; timolol and EMA401 are administered locally in a ratio of approximately 1:10 or approximately 1:1; cilazapril and EMA401 are administered locally in a ratio of approximately 1:10 or approximately 1:1; cilazapril and propranolol are administered locally in a ratio of approximately 1:1; ramipril and propranolol are administered locally in a ratio of from approximately 1:1; or trandolapril and timolol are administered locally in a ratio of approximately 4:1. In other embodiments, the combinations of compounds approximately at the ratios as specified in Table 6 herein after may be administered locally.

As mentioned herein before, in one embodiment, the invention provides a first-in-line method/medicament for early treatment or intervention in the progression of hemangioma. The inventors also contemplate it may provide a method/medicament for the treatment of rebound hemangiomas and/or a medicament for the secondary treatment for harder-to-treat hemangiomas that do not adequately respond to oral beta-blocker (eg propranolol) therapy alone, for example. Where a lesion is not severe, the invention may use an ACE inhibitor alone (or in one embodiment an ATIIR2 antagonist alone) applied in a topical formulation to manage or control the growth of the lesion. In cases where a faster involution is required or it would otherwise be useful to augment the treatment, one may use an ACE inhibitor combined with a β-blocker to treat, manage, or control the growth of the lesion. In other embodiments, an ACEi may be combined with an ATIIR2 antagonist, or with an ATIIR2 antagonist and a beta-blocker. In another embodiment, a beta-blocker and an ATIIR2 antagonist may be combined. In another embodiment, where the growth of a lesion is very severe, or the treatment response to local administration is lower or slower than desired, local administration in accordance with the invention may be combined with systemic administration of a suitable agent, such as an ACEi, beta-blocker and/or ATIIR2. In one embodiment, local administration in accordance with the invention is combined with systemic administration of a beta-blocker and/or ACEi. In one embodiment, it is combined with oral administration of an agent such as a beta-blocker and/or ACEi. Skilled persons will readily appreciate appropriate dosages and formulations of agents for systemic administration, having regard to the information contained herein and published information on systemic treatment of hemangiomas. However, by way of example, an oral beta-blocker preparation such as propranolol 3.75 mg/mL solution or Hemangeol® solution, or an oral ACE inhibitor solution such as a cilazapril solution or a captopril solution could be used. The inventors contemplate this providing a further synergistic response.

In certain embodiments of the invention, a combination of at least two of a beta-blocker, an ACEi and an ATIIR2 antagonist are administered systemically, preferably orally, to a subject. In one embodiment, compositions are preferably administered to provide a daily dose of active agent(s) from approximately 0.01 mg/kg (or approximately 0.1 mg/kg) to approximately 100 mg/kg of each individual agent to a subject. In one embodiment, a combination of at least two of a beta-blocker, an ACEi and an ATIIR2 antagonist are administered systemically by injection, for example subcutaneous, intramuscular, or intravenous injection, to provide a daily dose of active agent(s) from approximately 0.01 mg/kg (or approximately 0.1 mg/kg) to approximately 100 mg/kg of each individual agent to a subject.

Kits

The active agents (ACEI, beta-blocker and/or ATIIR2 antagonists) and compositions comprising same may be supplied and used in the form of a kit for the treatment of hemangioma. Such kits will comprise at least one and preferably a combination of active agents in one or more suitable containers. The agents may be formulated in a pharmaceutical composition ready for direct administration to a subject. Alternatively, the kit may comprise one or more active agent in one container and a pharmaceutical carrier composition in one or more other containers; the contents of each container being mixed together prior to administration. The kit may also comprise additional agents and compositions in further separate containers as may be necessary for a particular application. Further, kits of the invention can also comprise instructions for the use and administration of the components of the kit. Any container suitable for storing and/or administering a pharmaceutical composition may be used in a kit of the invention. Suitable containers will be appreciated by persons skilled in the art. By way of example, such containers include vials and syringes. The containers may be suitably sterilised and hermetically sealed.

### EXAMPLES

All inhibitors used in the following examples, with the exception of SMM02, were purchased from either Sigma Aldrich (www.sigmaaldrich.com), AK Scientific (www.aksci.com), MedChemExpress (www.medchemexpress.com), or MedKoo (www.medkoo.com). SMM02 (L-159,686, (S)-1,4-bis(N,N-diphenylcarbamoyl)piperazine-2-carboxylic acid) was synthesised as previously described in U.S. Pat. No. 5,292,726.

### Example 1: In Vitro Cell Culture Model of Infantile Hemangioma

An in vitro cell culture model of infantile hemangioma was developed to assess the impact of renin-angiotensin system inhibitors on cellular proliferation. A primary cell line of proliferating infantile hemangioma cells, including haemangioma stem cells, was derived from a tissue biopsy obtained from patients during the proliferating phase of the disease. The primary cell lines are cultured in 25 cm² flasks using standard culture media and conditions. The cultured cells are used to seed 96-well plates with a seed cell concentration of 2500 or 5000 cells per well, with additional culture media (200 μL). All studies are performed in triplicate.

The plates are incubated overnight to allow cell adherence to the wells. The wells are photographed to confirm cell adherence and confluency. The appropriate drug(s) to be examined are subsequently added to the wells. After 24 hours, one set of plates (experimental and blank) is stopped. The media is aspirated from each well and stored frozen for possible future analyses. Media is added to the wells and an MTT determination is undertaken according to standard protocols. This is repeated at 48 and 72 hours.

From the triplicate incubations of each drug concentration at each time, as well as for all controls, the mean±standard deviation is calculated. The net survival (optical density value) is calculated by subtracting the appropriate blank mean value from each cell culture. From the MTT data, the percentage of survival of cells in the presence of each concentration of drug after each time of culturing is determined.

### Example 2: Effect of Beta Blockers and ACE Inhibitors on Haemangioma Cell Proliferation in Patient-Excised Proliferating Infantile Haemangioma Cells In Vitro

Using the in vitro model of proliferating infantile haemangioma (Example 1), the effect of locally administered beta blockers and ACE inhibitors (ACEi) was investigated using timolol and cilazapril as a model beta blocker and ACE inhibitor respectively. This model recapitulates the process of locally administering the drug directly to the hemangioma microenvironment, which is representative of diffusing the drug through skin to reach the target cells.

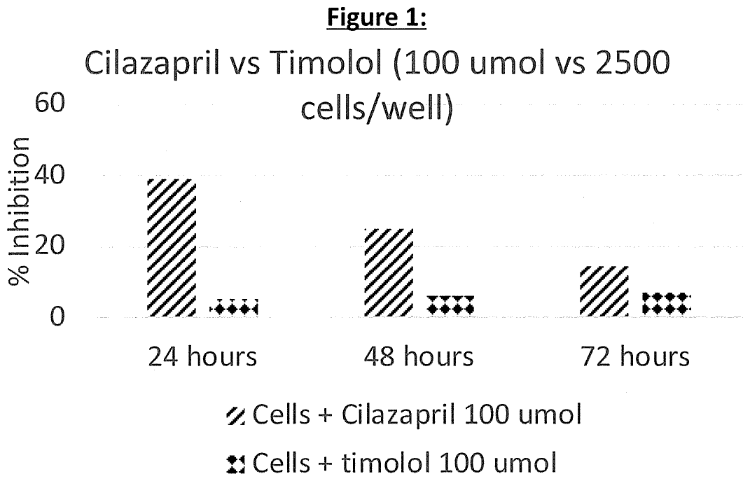

A single dose of timolol or cilazapril (100 μM) was added to the primary cell line containing haemangioma cells and stem cells, and cell viability was subsequently assessed by MTT at 24, 48, and 72 hours. Surprisingly, a significant 7-fold increase in the inhibition of haemangioma cell proliferation was observed after 24 hours of drug exposure to cilazapril compared to timolol (FIG. 1). This is in contrast to the current state-of-knowledge and clinical trial observations which report that ACE inhibitors are less effective than beta blockers at treating infantile haemangioma. Furthermore, these results show that high local concentrations of timolol alone have little effect on the attenuation of haemangioma tissue and support the limited clinical efficacy of current topically applied beta blocker products and formulation.

The positive results seen for local administration of cilazapril are also surprising due to the fact that it is a prodrug. It is well understood in the art that ACEi prodrugs require activation through hepatic biotransformation, which suggests that systemic administration would be the only effective means of delivery.

The observations interestingly demonstrate that, while the single dose of ACE inhibitor cilazapril maintains a greater inhibitory effect on the proliferation of IH cells than beta blocker timolol, the inhibitory effect is consistent with timolol but diminishes over time with cilazapril, suggesting that sequential dosing would be beneficial to help maintain constant inhibition.

In summary, this surprisingly demonstrates that ACE inhibitors are significantly more effective than beta blockers at inhibiting proliferation of haemangioma tissue. Therefore, ACE inhibitors may be more effective at treating proliferating haemangiomas when locally applied to the hemangioma, for example through topical application, including ACE inhibitors which are in the form of prodrugs. These findings inform a therapeutic dosing regimen for a topical ACE inhibitor product.

### Example 3: Effect of Beta Blocker and ACE Inhibitor Combinations of Haemangioma Cell Proliferation

As beta blockers and ACE inhibitors produced dramatically different inhibition profiles, the combination of an ACE inhibitor and a beta blocker on the inhibition of proliferating haemangioma cells was investigated using the in vitro haemangioma culture model (Example 1) and cilazapril and timolol as a model ACE inhibitor and beta blocker respectively.

Cilazapril and timolol were incubated with proliferating haemangioma cells at various low drug concentrations (0.1-10 μM), drug:drug ratios of 1:1 and 10:1, as either a single or daily dose, and with a haemangioma cell concentration of 2500 or 5000 cells/well. Cell viability was assessed at 24, 48, and 72 hours via MTT.

Surprisingly, the combination of cilazapril and timolol produced a greater and prolonged inhibition of haemangioma cell proliferation, which can be achieved at lower drug concentrations, particularly with daily dosing (FIG. 2, 5000 cells/well). Furthermore, even at lower drug concentrations (10 μM), prolonged and significant inhibition of haemangioma cell proliferation was achieved with a higher initial cell concentration (5000 cells/well). This was not achieved with single drugs.

In summary, these results demonstrate that the combination of an ACE inhibitor with a beta blocker is substantially more effective at attenuating proliferating haemangioma and inform the manufacture of an effective topical treatment for this disease.

### Example 4: Effect of Combinations of ACE Inhibitors, Beta Blockers, and Angiotensin Receptor Blockers on Haemangioma Cell Proliferation Across Multiple Patient Cell Lines

Further to the investigations described in Examples 1-3, the inhibitory effect of beta-blockers, ACE inhibitors, and angiotensin II type 1 (AT1) and type 2 (AT2) receptor inhibitors on haemangioma cell proliferation was confirmed across multiple proliferating haemangioma cells lines derived from patient-biopsies obtained from five patients between two and ten months of age (Table 2).

TABLE 2

| | Age at | |
|---|---|---|
| Sample | Surgery | |
| ID | (months) | Location |
| 15-034 * | 2 | Scalp |
| 15-176 | 7 | Ear |
| 18-017 | 10 | Post Auricular |
| 19-255 II | 4 | Neck/Clavicle |
| 19-275 | 3 | Scapula |

* Results across multiple experiments

The experimental protocol was performed as per Example 1. Briefly, cells were seeded in 96-well plates at a concentration of 2500 cells/well in triplicate for each drug and drug combination. Drug(s) were added to each well 24 hours after seeding and re-dosed every 24 hours. Cell viability was assessed by MTT assay after 24, 48, and 72 hours. All plates were performed in triplicate. Drugs were assessed alone and in combination as described in Table 3. Timolol was used as a model beta-blocker (BB), cilazapril as a model ACE inhibitor (ACEi), losartan as a model angiotensin II type 1 receptor inhibitor (AT1Ri), and EMA401 and SMM02 (L-159686) as model AT2Ri's. A summary of the results at 72 hours is shown in Table 4.

TABLE 3

| | Timolol | Cilazapril | Losartan | EMA401 | SMM02 |
|---|---|---|---|---|---|
| Timolol | N | Y | Y | Y | Y |
| Cilazapril | Y | N | Y | Y | Y |
| Losartan | Y | Y | N | N | N |

TABLE 4

| | | | Estimated |
|---|---|---|---|
| | | Individual Responders | Optimal |
| | | at 72 Hours with | Dosage |
| | | ≥30% Inhibition vs. | Range in |
| | Results Summary | Cells + Adjuvant | vitro (μM) |
| Timolol | Responder | 10 μM - 4/5 | 1-10 |
| | | 1 μM - 0/5 | |
| Cilazapril | Responder | 10 μM - 4/5 | 1-10 |
| | | 1 μM - 1/5 | |
| Losartan | Non-Responder | 10 μM - 0/5 | N/A |
| | | 1 μM - 0/5 | |
| EMA401 | Responder | 100 μM - 5/5 | 5-25 |
| | | 10 μM - 4/5 | |
| | | 1 μM - ¼ | |
| SMM02 | Responder | 100 μM - 5/5 | 1-10 |
| | | 10 μM - 5/5 | |
| | | 1 μM - 0/4 | |
| Timolol/Cilazapril | Responder/Responder | 10/10 μM - 5/5 | 1-10 & 1-10 |
| | | 10/1 μM - 4/5 | |
| | | 1/10 μM - 4/5 | |
| Timolol/Losartan | Responder/Non-Responder | 10/10 μM - 5/5 | 1-10 & 1-10 |
| | | 10/1 μM - 5/5 | |
| | | 1/10 μM - 0/5 | |
| Cilazapril/Losartan | Responder/Non-Responder | 10/10 μM - 2/5 | 1-10 & 1-10 |
| | | 10/1 μM - 3/5 | |
| | | 1/10 μM - 0/5 | |
| Timolol/EMA401 | Responder/Responder | 10/100 μM - 5/5 | 1-10 & 5-25 |
| | | 1/100 μM - 5/5 | |
| | | 10/10 μM - 5/5 | |
| Cilazapril/EMA401 | Responder/Responder | 10/100 μM - 5/5 | 1-10 & 5-25 |
| | | 1/100 μM - 5/5 | |
| | | 10/10 μM - 5/5 | |
| Timolol/SMM02 | Responder/Responder | 10/100 μM - 5/5 | 1-10 & 1-10 |
| | | 1/100 μM - 5/5 | |
| | | 10/10 μM - 5/5 | |
| Cilazapril/SMM02 | Responder/Responder | 10/100 μM - 5/5 | 1-10 & 1-10 |
| | | 1/100 μM - 5/5 | |
| | | 10/10 μM - 5/5 | |

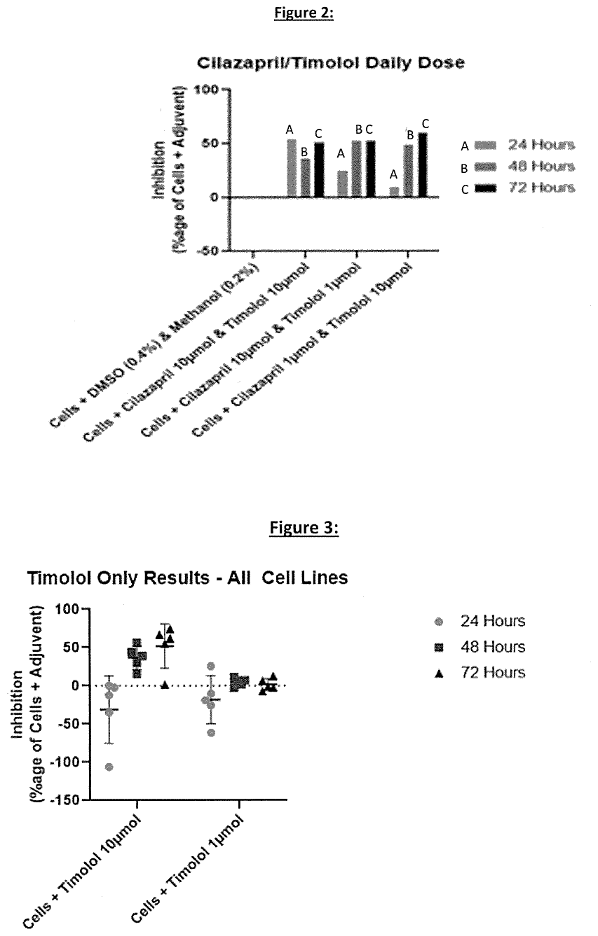

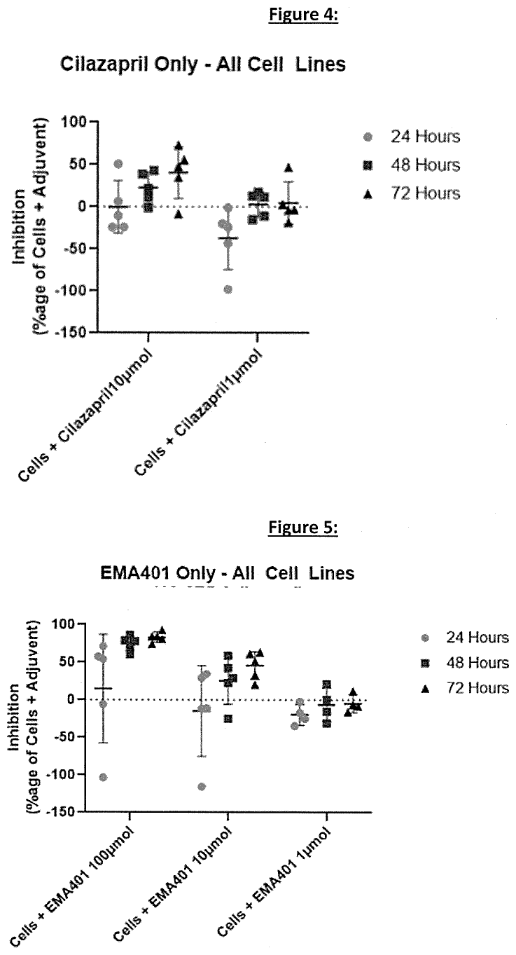

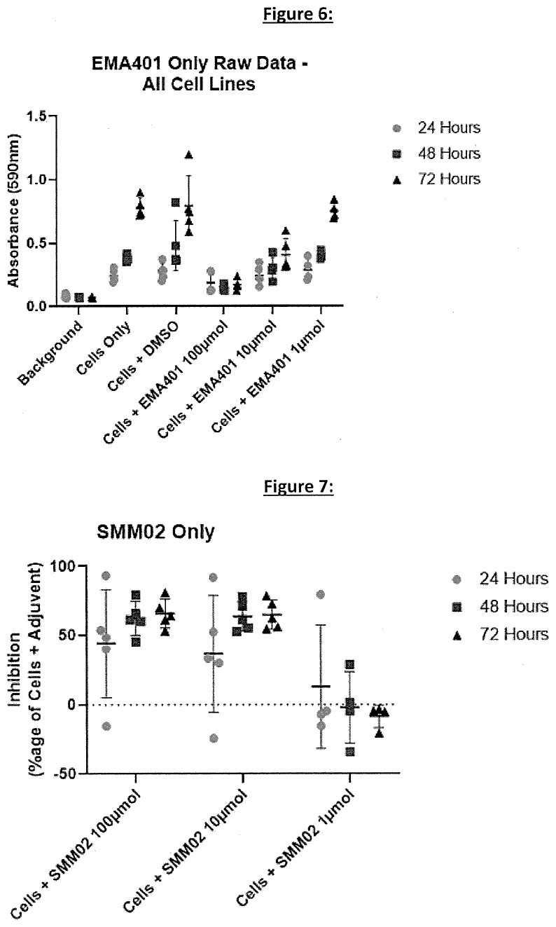

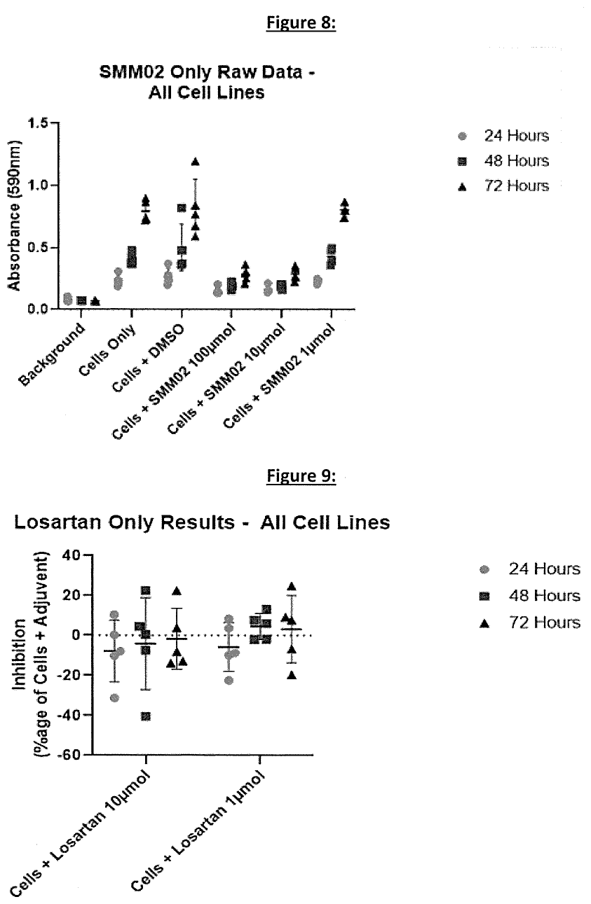

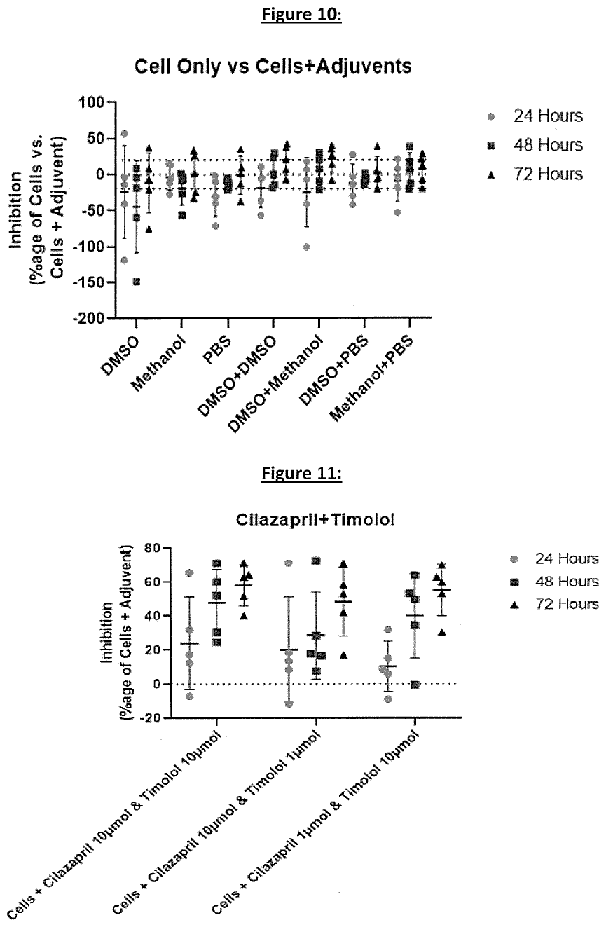

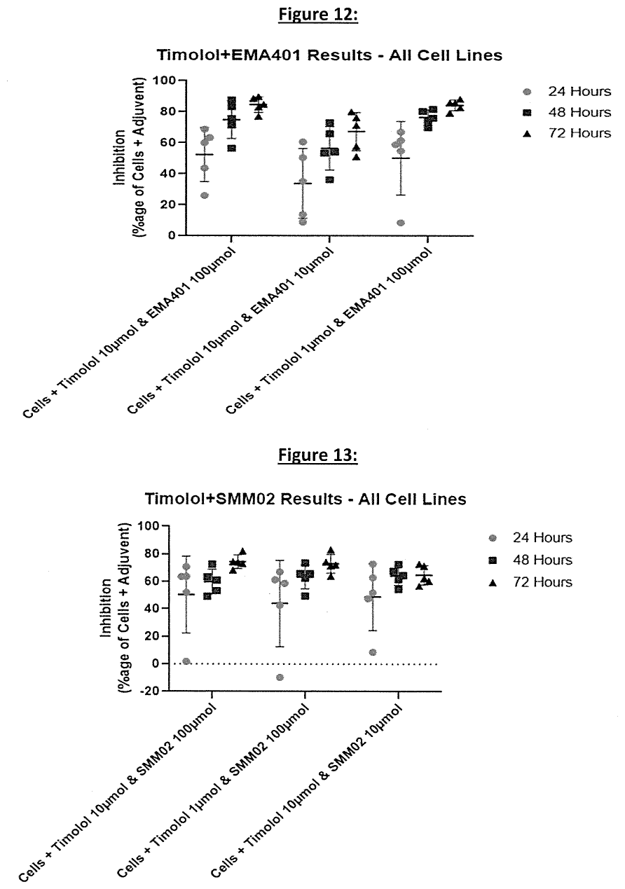

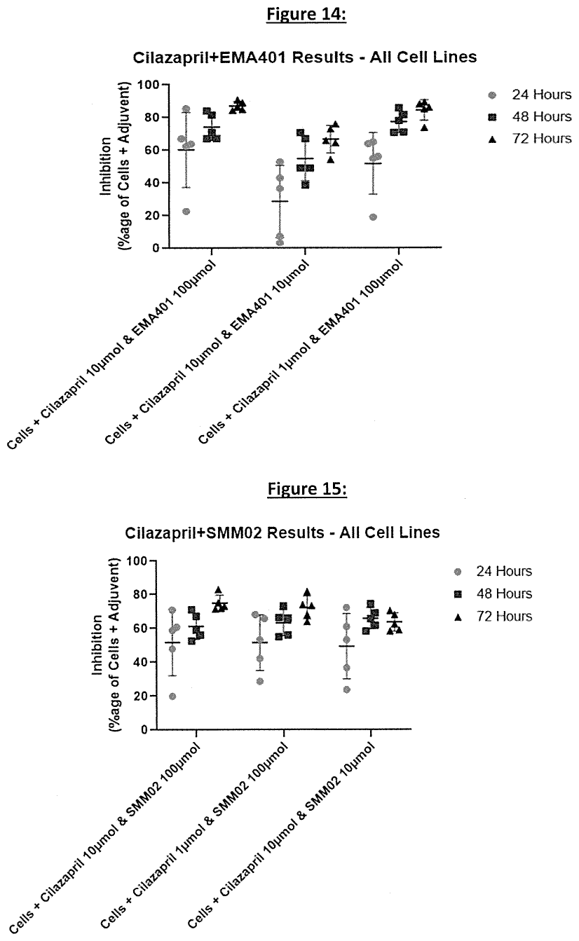

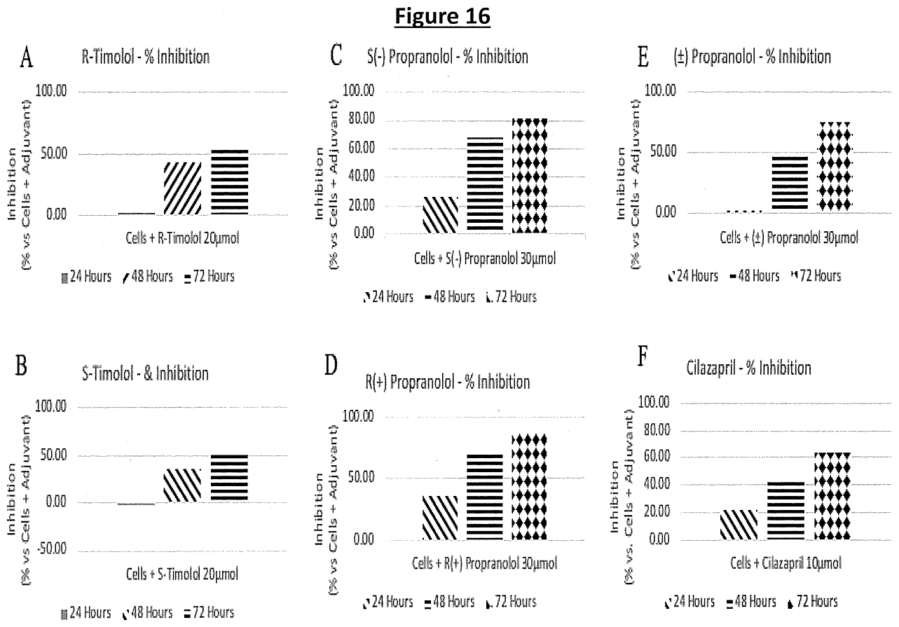

All five patient-derived haemangioma cell lines were significantly inhibited by the application of: a beta-blocker (timolol, FIG. 3)); an angiotensin converting enzyme inhibitor (cilazapril, FIG. 4); an angiotensin II type 2 receptor inhibitor (EMA401 (FIGS. 5 and 6) and SMM02 (FIGS. 7 and 8)); but not by an angiotensin II type 1 receptor inhibitor (losartan, FIG. 9). Control wells containing cells only and cells and the adjuvant used to dissolve the appropriate drug candidate showed no change in cell viability (FIG. 10). Furthermore, the combination of: a beta blocker (timolol) and an angiotensin converting enzyme inhibitor (cilazapril) (FIG. 11), a beta blocker (timolol) and an angiotensin II type 2 receptor inhibitor (EMA401 (FIG. 12) and SMM02 (FIG. 13)), and an angiotensin converting enzyme inhibitor (cilazapril) and an angiotensin II type 2 receptor inhibitor (EMA401 (FIG. 14) and SMM02 (FIG. 15)) was found to be superior to the use of single agents alone and appear to enhance the inhibitory effect on proliferating haemangioma cells through synergistic action. The combinations produce a better response at the same or lower doses compared to the use of single agents.

FIGS. 3-15 show both the average response as well as the variation in response between five primary hemangioma cell lines derived from different patients during the proliferating phase when treated with either single agents or combinations of agents. The degree of response variation, particularly in the case of timolol, reflects the variation clinically observed between patients when treated with oral or topical beta blocker therapy or oral ACE inhibitors. However, overall, results (FIGS. 3 to 15) indicate a synergistic effect is observed for the various drug combinations and that treatment with combinations of agents is observed to have to have a more consistent response between primary hemangioma cell lines when concentrations >1 μM are used. In addition, the inhibitory effect of each agent improved over time, suggesting that it may be advantageous to use multiple or daily doses may to treat a patient. This is particularly evident in the 24 hour time points which display large variation in response between each patient-derived cell line. It should be noted that negative inhibition values (as observed for some cell lines at different time points, or when the lowest concentration of agent (1 μM) is used) should be interpreted as zero or negligible inhibitory effect.

### Example 5: Investigation into Combination of R- and S-Beta Blockers with ACE Inhibitors, Beta Blockers on Hemangioma Cell Proliferation

Further investigations were performed using ACE inhibitors and beta blockers alone and in combination to establish their effects at low individual concentrations, and to investigate the effect of different beta blocker enantiomers.

The experimental protocol was performed as per Example 1. Briefly, cells were seeded in 96-well plates at concentrations of 2500 cells/well in triplicate for each drug and drug combination. Drug(s) were added to each well 24 hours after seeding and re-dosed every 24 hours. Cell viability was assessed by MTT assay after 24, 48, and 72 hours. All plates were performed in triplicate. Drugs were assessed alone and in combination, at concentrations between 1 and 30 μM (FIGS. 16-18). Timolol and propranolol were used as model beta blockers and cilazapril as a model ACE inhibitor.

The effect of R-timolol, S-timolol, R-propranolol, S-propranolol, and R/S-propranolol were investigated alone and in combination with cilazapril. R- and S-propranolol was found to be superior to R- and S-timolol at attenuating hemangioma cell proliferation, particularly at low concentrations of 1-5 μM. However, surprisingly it was found that there is little difference between the ability of R-beta blockers and S-beta blockers to inhibit proliferating hemangioma cells (FIG. 16). It was also noted that surprisingly, the individual isomers, R- and S-propranolol were as effective alone as the racemic mixture which is currently prescribed for the treatment of proliferating infantile hemangioma requiring systemic therapy under the trade name Hemangiol®. This is a significant finding as the R-enantiomers of timolol and propranolol do not exert blood pressure effects to the same degree as the S-enantiomers.

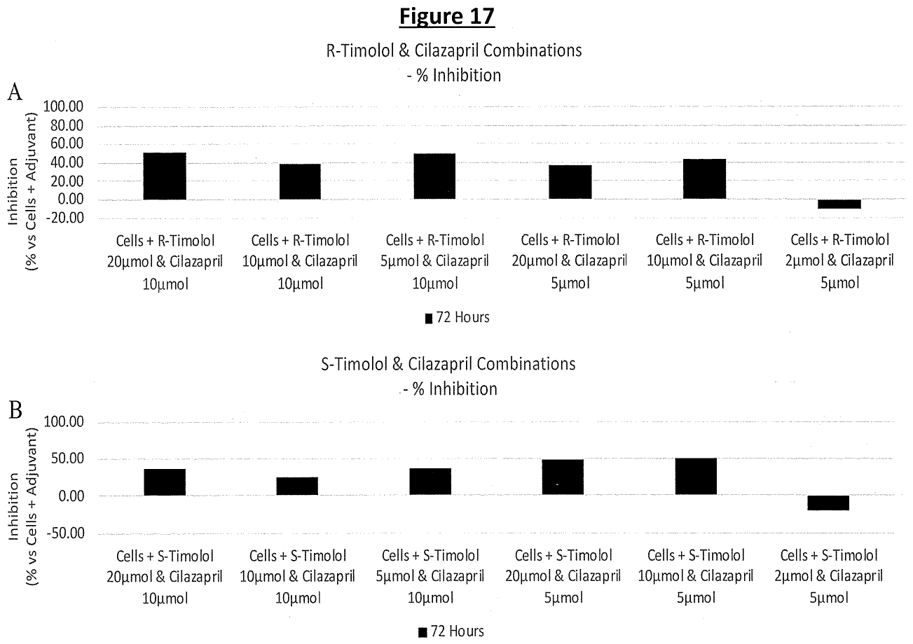

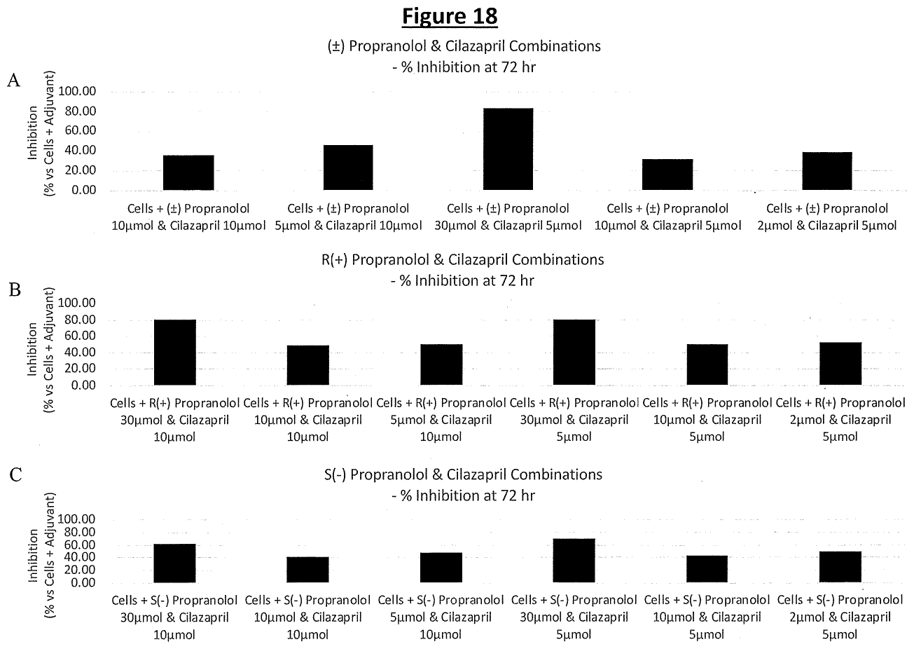

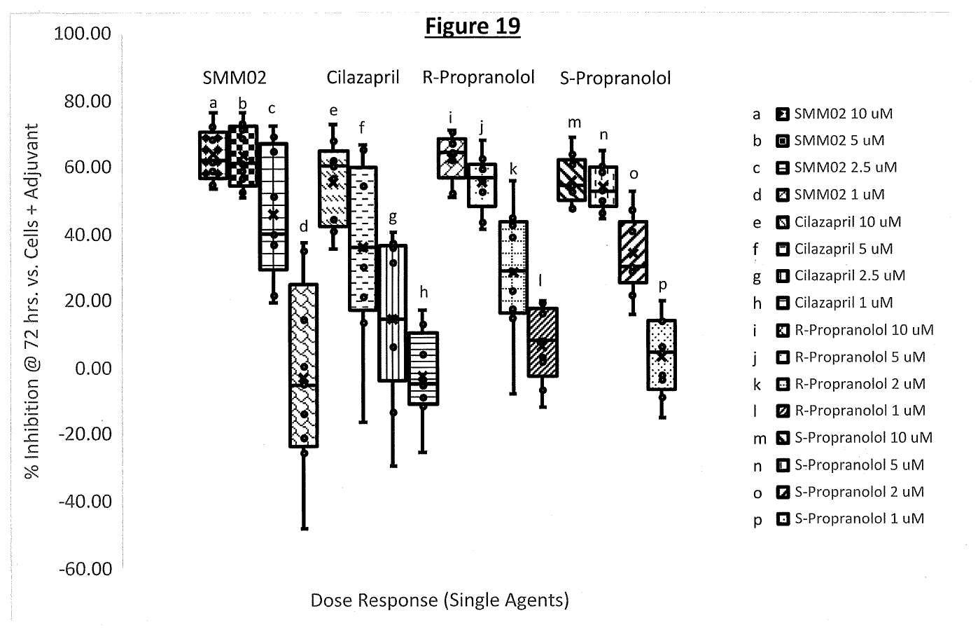

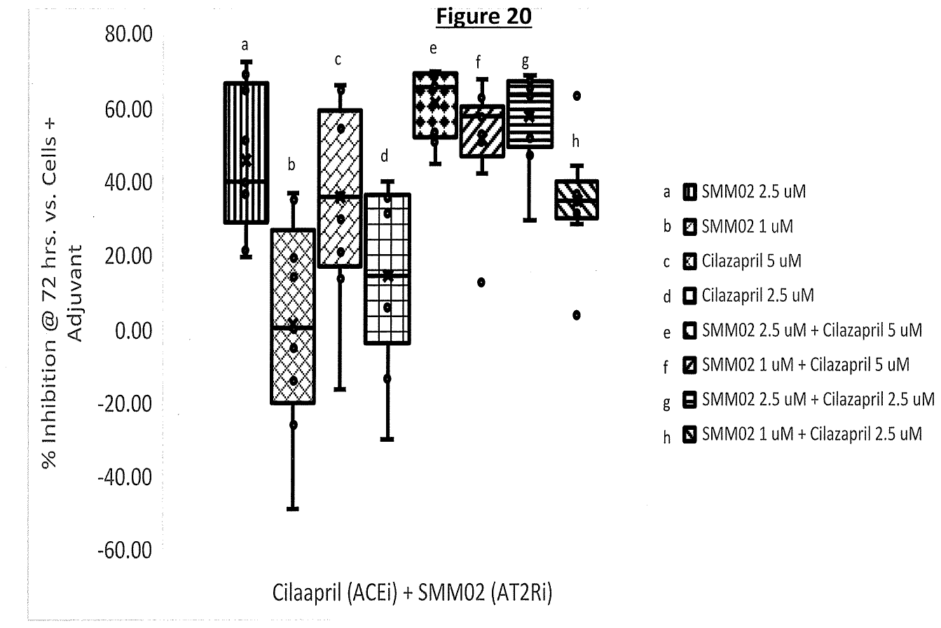

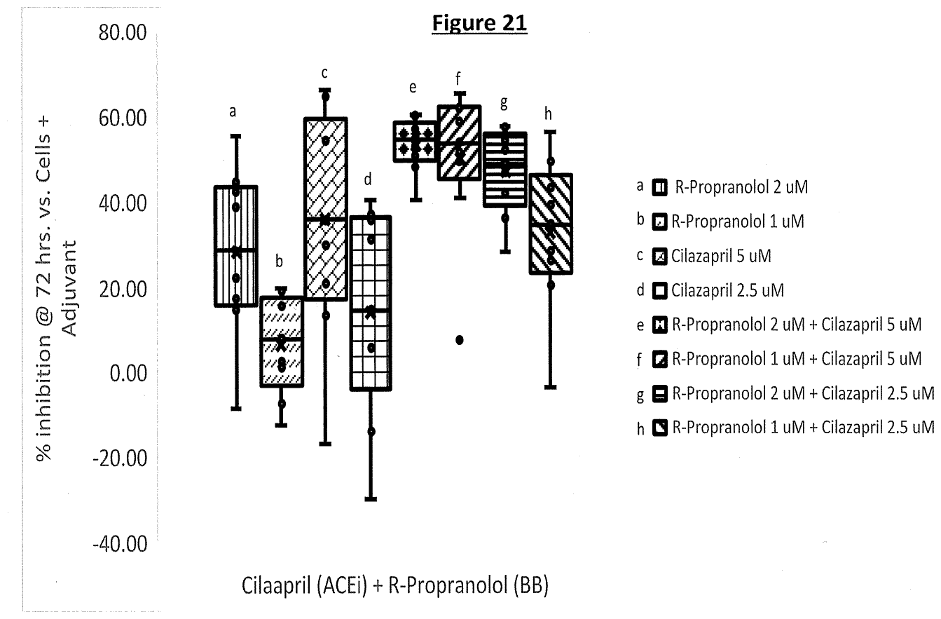

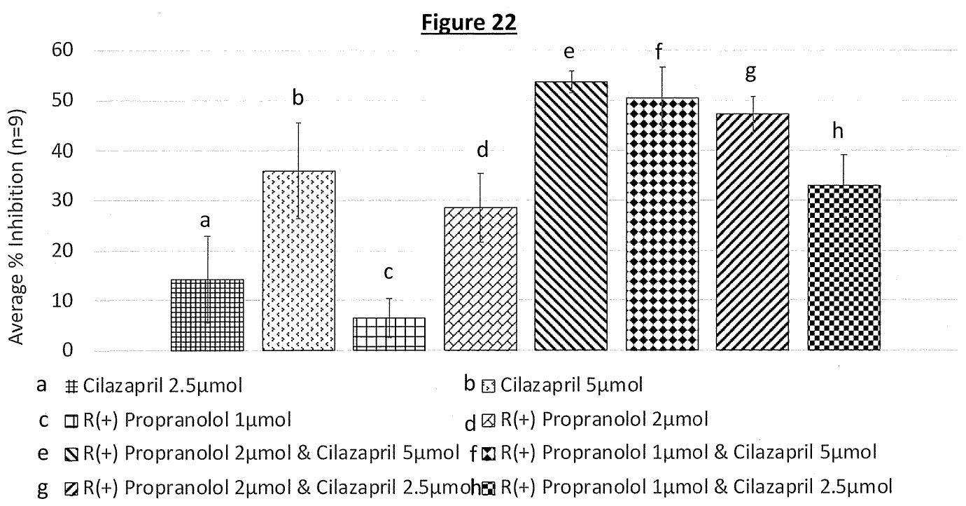

The effect of beta blocker enantiomer on hemangioma proliferation in combination with an ACE inhibitor was subsequently investigated. Cilazapril was used as an example ACE inhibitor. The combination of a beta blocker with cilazapril provided a greater effect on the inhibition of proliferating hemangioma cells than the individual agents (as seen for example in FIG. 17 and FIG. 18, 72 hours). The combinations R-timolol/cilazapril and 5-timolol/cilazapril showed a similar potency, as did the combinations R-propranolol/cilazapril, 5-propranolol/cilazapril, and R/S-propranolol/cilazapril. The beneficial therapeutic effect of an R-beta blocker with an ACE inhibitor is a surprising and a clinically significant finding as it demonstrates that these agents may be combined without increasing the risk of cardiovascular side effects.

### Example 6: Synergistic Effects Between Combinations of ACE Inhibitors, Beta Blockers and Angiotensin II Type 2 Receptor Blockers for Inhibiting Proliferating Hemangioma

A study was undertaken using combinations of ACE inhibitors, beta blockers and angiotensin II type 2 receptor blockers to inhibit proliferating hemangioma cells. The inhibition of hemangioma cell proliferation by these agents alone and in combination was confirmed in an extended investigation using multiple patient-derived primary hemangioma cell lines obtained via biopsy during the proliferating phase (n=9). Patient histories pertaining to each cell line is described in Table 5.

TABLE 5
History of patient biopsies used for primary cell culture.

| Cell | | Age at | |
|---|---|---|---|
| Line | Sample | Surgery | |
| No. | ID | (months) | Location |
| 1 | 15-034* | 2 | Scalp |
| 2 | 19-255 II | 4 | Neck/Clavicle |
| 3 | 19-275 | 3 | Right Scapula |
| 4 | 15-208 | 3 | Head & Neck |
| 5 | 17-092 | 5 | Head & Neck |
| 6 | 17-183 | 6 | Head & Neck |
| 7 | 19-229 II | 3 | Head & Neck |
| 8 | 17-209 I | 4 | Trunk |
| 9 | 20-108 | 5 | Trunk |

* Results across multiple experiments

The experimental protocol was performed as per Example 1. Briefly, cells were seeded in 96-well plates at concentrations of 2500 cells/well in triplicate for each drug and drug combination. Drug(s) were added to each well 24 hours after seeding and re-dosed every 24 hours. Cell viability was assessed by MTT assay after 24, 48, and 72 hours. All plates were performed in triplicate. Drugs were assessed alone and in combination, as described in Table 6. Propranolol was used as a model beta blocker, cilazapril as a model ACE inhibitor, and SMM02 (L-159686) as a model AT2Ri. Synergy was measured using the CompuSyn open-source software protocol for quantifying synergism and antagonism that uses the median-effect equation and mass-action laws to generate a combination index (www.compusyn.com). Synergism, additive effects and antagonism are defined as a CI<1, CI=1, and CI>1, respectively.

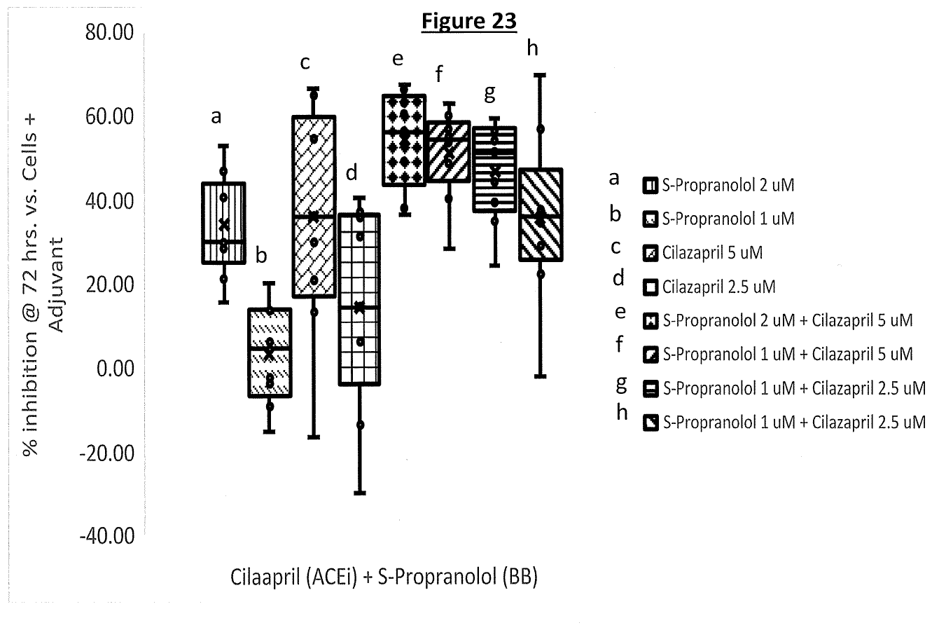

ACE inhibitor cilazapril and beta blockers R-propranolol and S-propranolol showed a typical dose-response across all cell lines between 1 and 10 μM (as seen for example in FIG. 19, 72 hours). Interestingly, the AT2R antagonist SMM02 demonstrated a non-linear dose-response across all cell lines, where little difference in the degree of inhibition is observed between 2.5 μM and 10 μM, suggesting more of a “threshold” or “saturation” effect, where maximal inhibition can be achieved once a minimum concentration has been achieved. Furthermore, treatment of cells with the AT2R antagonist SMM02 as a single agent demonstrated a more consistent inhibitory response across all patient cell lines than other agents when treatment occurred above the threshold concentration of 2.5 μM. These results are consistent with the observations of Example 4 (FIGS. 3-8).

TABLE 6
Drug combinations, concentrations and ratios tested.

| | Conc 1 | Conc 2 | Conc 3 | Conc 4 |
|---|---|---|---|---|
| (±) Propranolol | 10 μM | 5 μM | 2 μM | 1 μM |
| R(+) Propranolol | 10 μM | 5 μM | 2 μM | 1 μM |
| S(−) Propranolol | 10 μM | 5 μM | 2 μM | 1 μM |
| SMM02 | 10 μM | 5 μM | 2.5 μM | 1 μM |
| Cilazapril | 10 μM | 5 μM | 2.5 μM | 1 μM |
| Cilazapril + R(+) | 5/2 | 5/1 | 2.5/2 | 2.5/1 |
| Propranolol | 2:2 | 5:1 | 5:4 | 5:2 |
| Cilazapril + S(−) | 5/2 | 5/1 | 2.5/2 | 2.5/1 |
| Propranolol | 2:2 | 5:1 | 5:4 | 5:2 |
| Cilazapril + SMM02 | 5/2.5 | 5/1 | 2.5/2.5 | 2.5/1 |
| | 2:1 | 5:1 | 1:1 | 5:2 |
| SMM02 + R(+) | 5/2 | 5/1 | 2.5/2 | 2.5/1 |
| Propranolol | 5:2 | 5:1 | 5:4 | 5:2 |
| SMM02 + S(−) | 5/2 | 5/1 | 2.5/2 | 2.5/1 |
| Propranolol | 5:2 | 5:1 | 5:4 | 5:2 |

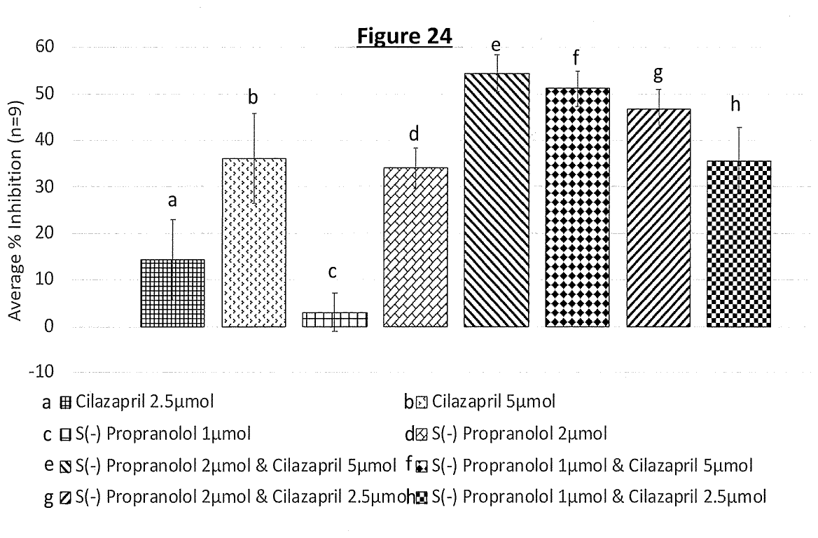

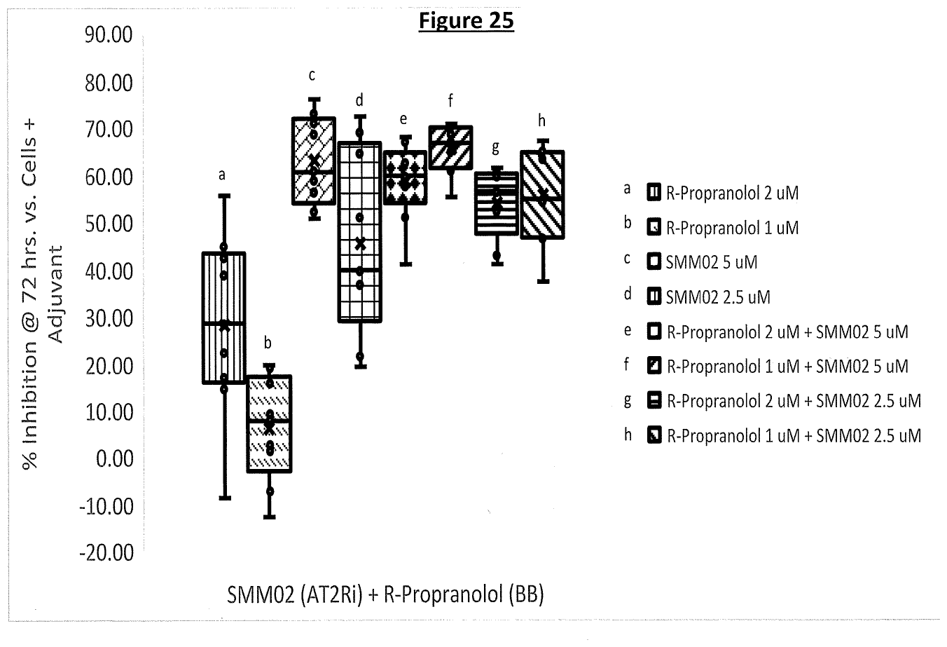

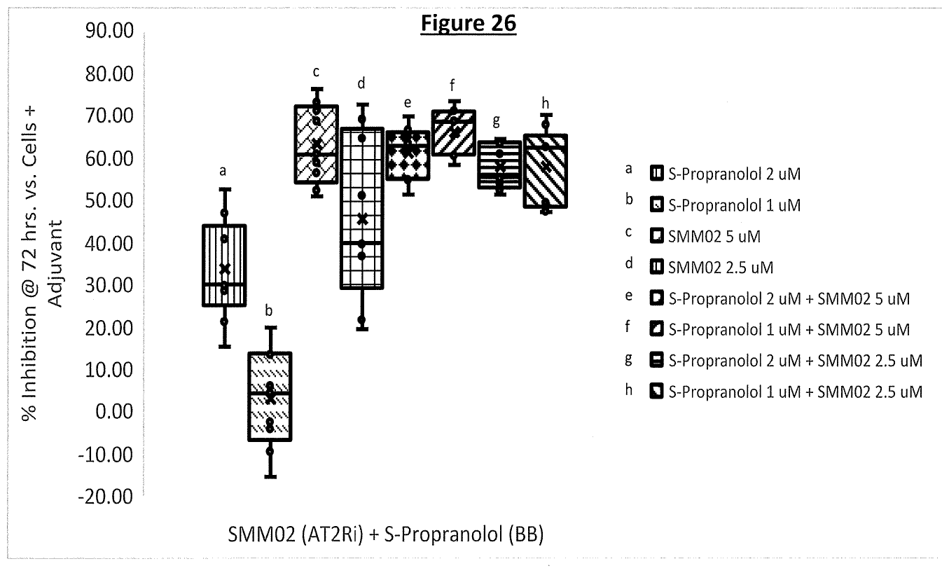

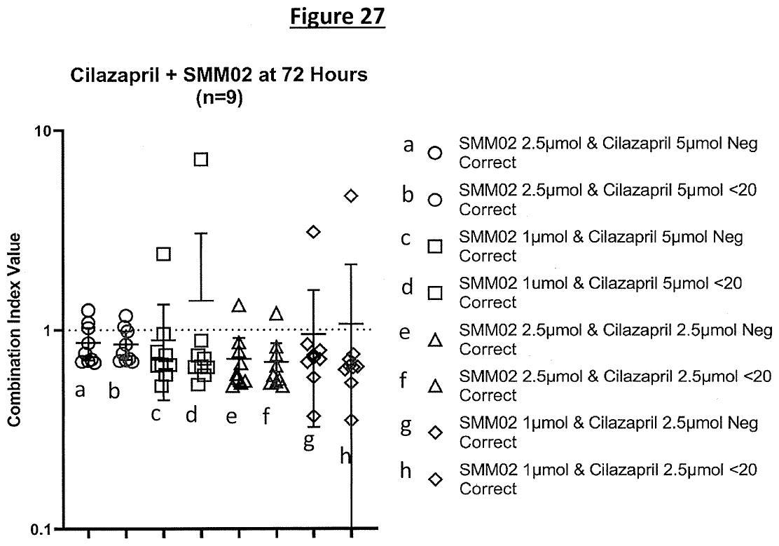

In general, the combinations of inhibitions (cilazapril+SMM02; cilazapril+R-propranolol; cilazapril+S-propranolol; R-propranolol+SMM02; and S-propranolol+SMM02) all demonstrated higher levels of inhibition than the corresponding concentrations of single agents, particularly when lower concentrations of agents were used (as seen for example in FIGS. 19-26, 72 hours). Surprisingly, the combinations of inhibitors also demonstrated more consistent levels of inhibition between different patient-derived cell lines. In contrast, inhibition with single agents tended to have a higher degree of variability between cell lines. These results provide unique insights which inform the development of an effective fixed-dose combination therapy for the topical treatment of haemangiomas and are consistent with the observations of Example 3 (FIGS. 3-15).

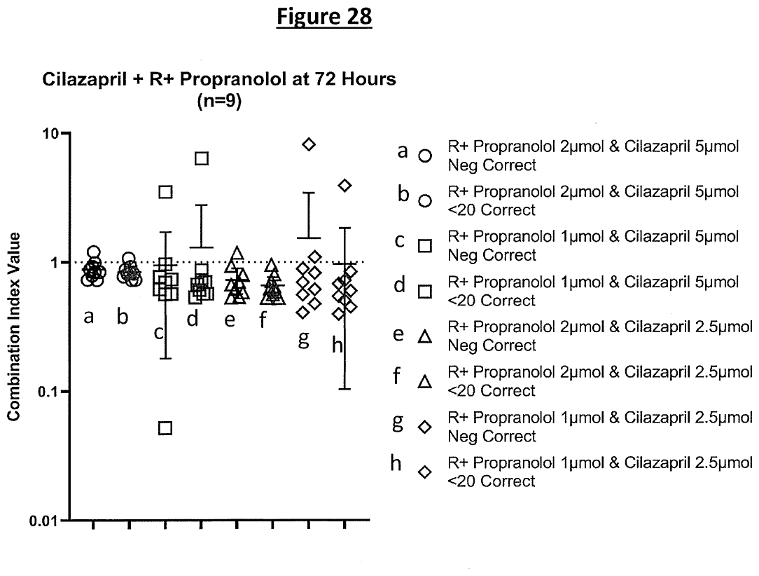

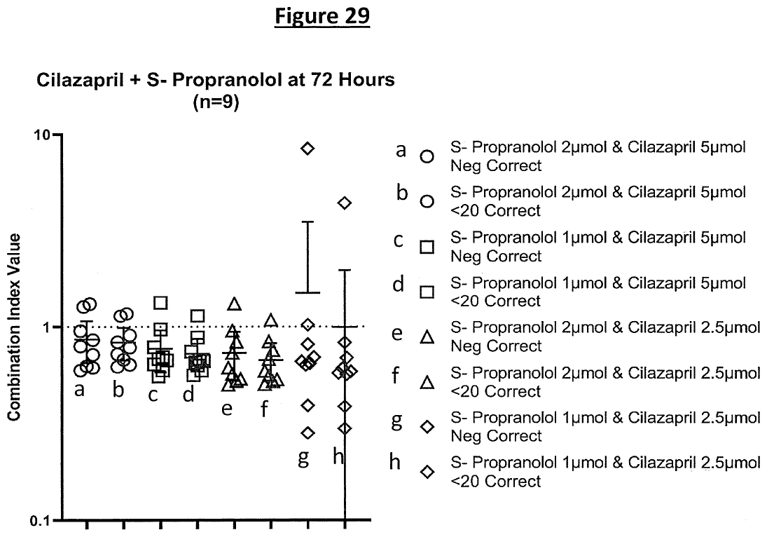

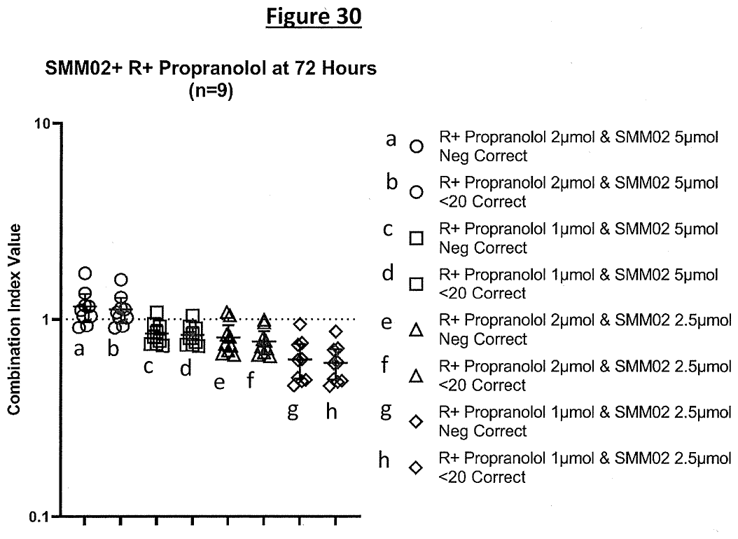

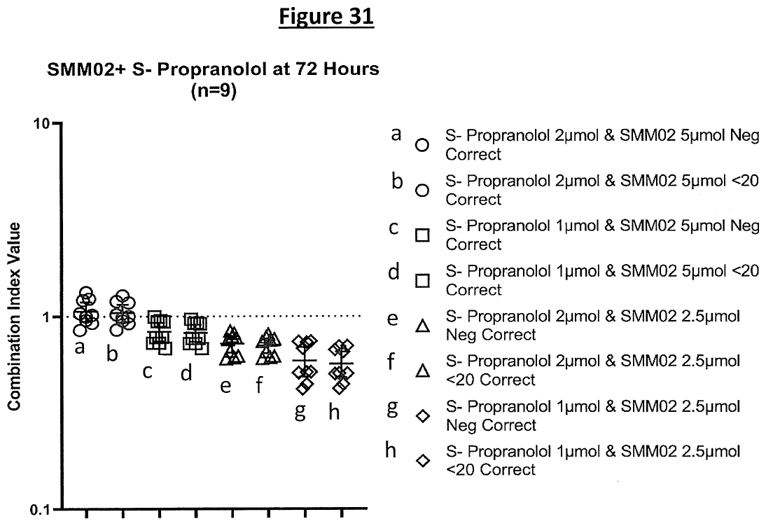

Surprisingly, all combinations (SMM02/cilazapril, R/S-propranolol/cilazapril, R-propranolol/cilazapril, S-propranolol/cilazapril, R/S-propranolol/SMM02, R-propranolol/SMM02, and 5-propranolol/SMM02) demonstrated a synergistic inhibition of proliferating hemangioma cells in vitro (FIGS. 27-31). This was most evident at the 72 hour time point (which is representative of a likely clinical situation in which multiple doses are administered to a subject over time) and at low individual concentrations.

Demonstrating a synergistic therapeutic benefit for inhibiting proliferating hemangioma when ACE inhibitors, Beta Blockers and AT2 receptor inhibitors are used is a significant finding and may provide substantial benefit for the treatment of infantile hemangioma. Of particular importance is the combination of R-propranolol and cilazapril, and R-propranolol with an AT2 receptor inhibitor (for example, SMM02 or EMA401). The former combination of R-propranolol with an ACE inhibitor, may confer synergistic benefit in the treatment of infantile hemangioma, reducing the dosage required for treatment and minimizing the potential for cardiovascular side effects. The latter combination of R-propranolol with an AT2 receptor inhibitor (for example, SMM02 or EMA401) may confer a synergistic therapeutic benefit at low dose without any cardiovascular side effects, as neither R-propranolol or AT2 receptor inhibitors are known to regulate blood pressure to a significant degree.

These combinations, including the combination of cilazapril and propranolol also provided a significantly increased degree of inhibition, which was highly consistent between individual patient cell lines relative to the use of single agents. Based on these observations, the inventors contemplate that the combination of an ACE inhibitor and a beta blocker, the combination of an AT2 antagonist or a beta blocker, and the combination of an AT2 antagonist and an ACE inhibitor may offer more effective treatments and provide a more consistent response across patient populations than beta blockers alone. This may be particularly useful as a front-line treatment as a topical therapy to prevent the progression of small hemangiomas into large hemangiomas that cause permanent disfiguration, threaten life or function, and require invasive systemic or surgical treatment, or for the secondary treatment of rebound hemangiomas, or hemangiomas that do not completely respond to beta blocker therapy alone.

### Experiment 7: Investigating the Effect of Different ACEi's and BB's on Proliferating Hemangioma Cell Lines

Due to the synergistic inhibition of proliferating hemangioma cells observed with drug combinations containing the ACE inhibitor cilazapril and beta-blockers propranolol and timolol, a study was undertaken to investigate the impact of different ACE's and BB's on the inhibition of proliferating hemangioma cells.

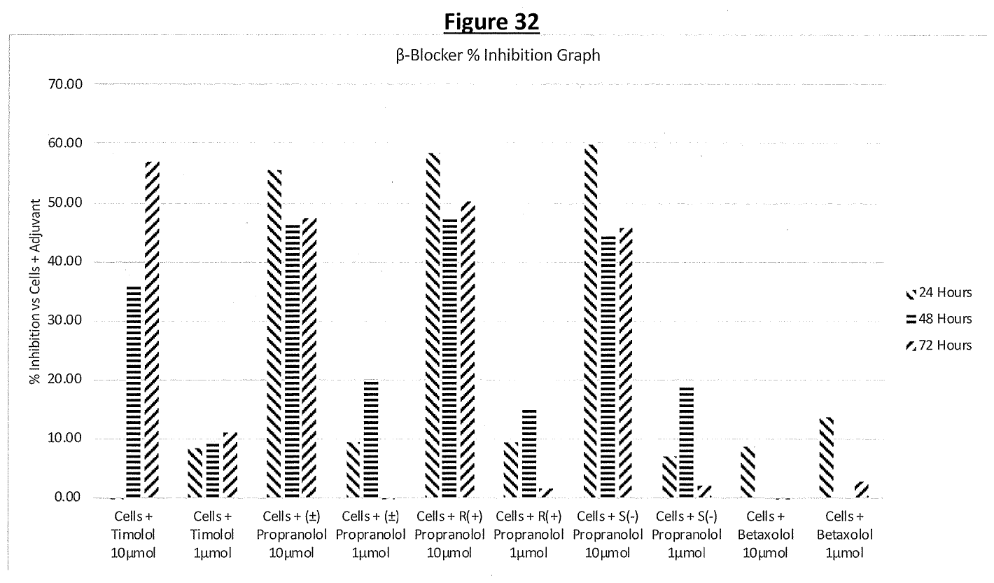

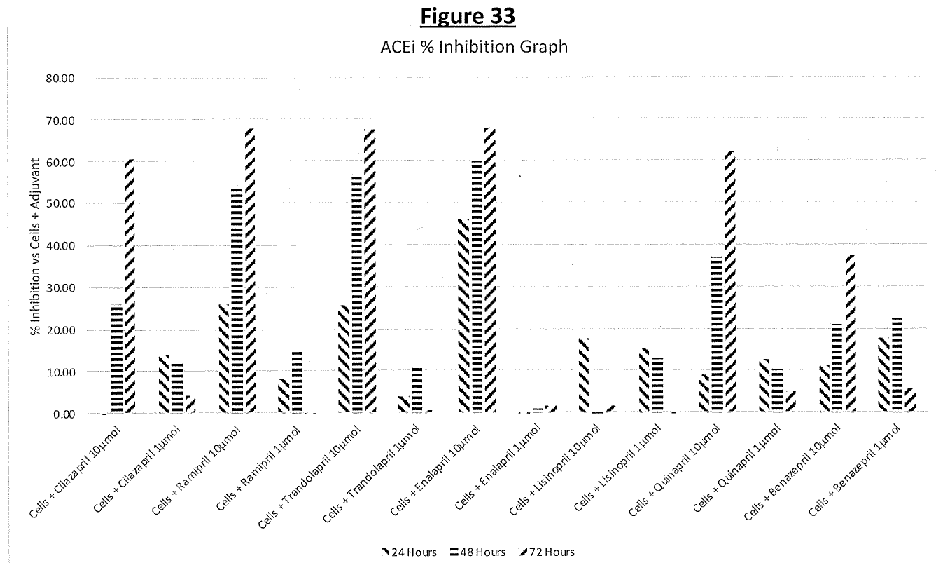

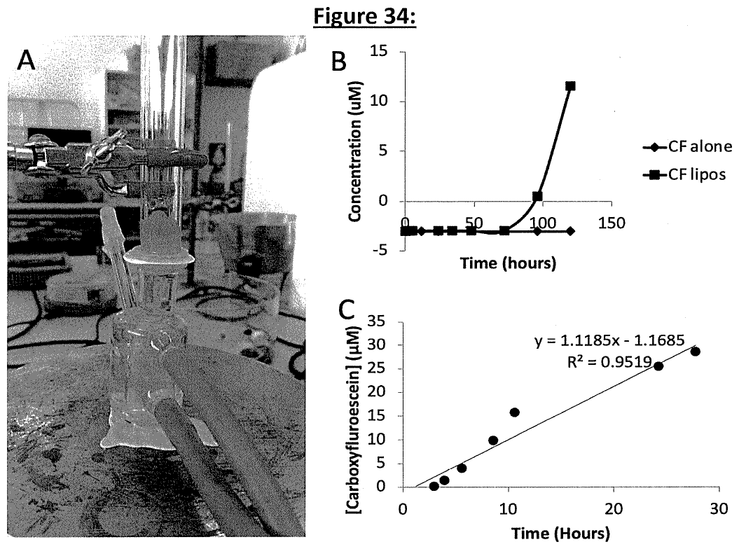

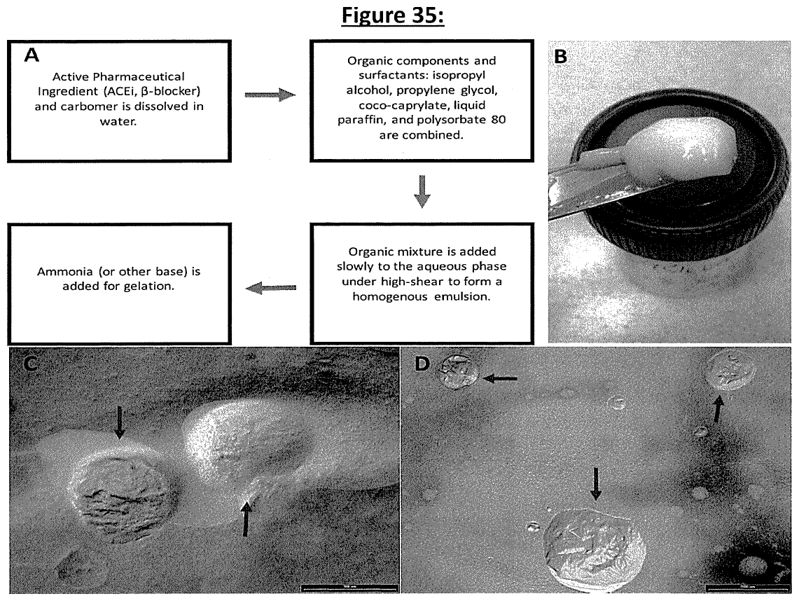

The experimental protocol was performed as per Example 1 using one primary hemangioma cell line derived from biopsies from individual patients during the proliferating phase. Briefly, cells were seeded in 96-well plates at concentrations of 2500 cells/well in triplicate for each drug and drug combination. Drug(s) were added to each well 24 hours after seeding and re-dosed every 24 hours. Cell viability was assessed by MTT assay after 24, 48, and 72 hours. All plates were performed in triplicate. Beta blockers used in the study were (±)-propranolol, R-propranolol, S-propranolol, timolol, and betaxalol. ACE inhibitors used in the study were cilazapril, ramipril, trandolapril, enalapril, lisinopril, and quinapril. A summary of results is provided in Table 7 and FIGS. 32-33.

Beta blockers timolol and propranolol, either as a racemic mixture or as single enantiomers, showed a consistent level of inhibition (45%-57% inhibition). Surprisingly however, the beta blocker betaxolol showed no response at low (1 μM) or high (10 μM) concentration (FIG. 32, Table 7). Without wishing to be bound by theory, the inventors contemplate that the difference in inhibition is due to the difference in receptor selectivity; both timolol and propranolol are non-selective beta blockers whereas betaxolol is a beta₁-selective beta blocker.

The ACE inhibitors cilazapril, ramipril, trandolapril, enalapril, and quinapril also showed a good and similar level of inhibition against proliferating hemangioma cells (FIG. 33, Table 7). Interestingly, these ACE inhibitors are all ethyl ester prodrugs of their active metabolites which are normally produced by first pass metabolism after oral ingestion. Furthermore, it is generally understood that the ethylester prodrug form of each ACE inhibitor possesses a lower chemical affinity for ACE when compared to the active carboxylic acid metabolite. In contrast, the non-prodrug ACE inhibitor lisinopril had no effect on proliferating hemangioma cells (FIG. 33, Table 7). This is a surprising result as it would be expected that using a non-prodrug form of an ACE inhibitor should produce a better, or at least equal effect.

These results suggest that non-selective beta blockers and ACE inhibitors in prodrug form are preferred to practise the present invention, or at least to produce an optimum result. Furthermore, as beta blockers propranolol and timolol, and their individual enantiomers, and ACE inhibitors cilazapril, ramipril, trandolapril, quinapril and enalapril display equivalent efficacy, the inventors contemplate that the invention can be practiced equivalently by interchanging combinations of these agents.

TABLE 7
Summary of results: effect of different beta-blockers and
ACE inhibitors on the inhibition of proliferating hemangioma cells at 72 hours.

| | Results | At 72 Hours >30% Inhibition vs. | Estimated Optimal |
|---|---|---|---|
| | Summary | Cells + Adjuvant | Dosage Range (μM) |
| Timolol | Responder | 10 μM - Response - 56.97% | 1-10 |
| | | 1 μM - No - 11.08% | |
| (±) Propranolol | Responder | 10 μM - Response - 47.46% | 1-10 |
| | | 1μM - No - <0% | |
| R(+) Propranolol | Responder | 10 μM - Response - 50.35% | 1-10 |
| | | 1 μM - No - 1.59% | |
| S(−) Propranolol | Responder | 10 μM - Response - 45.68% | 1-10 |
| | | 1 μM - No - 2.07% | |
| Betaxolol | Non- | 10 μM - <0% | 1-10 |
| | Responder | 1 μM - 2.80% | |
| Cliazapril | Responder | 10 μM - Response - 60.53% | 1-10 |
| | | l μM - No - 4.35% | |
| Ramipril | Responder | 10 μM - Response - 68.09% | 1-10 |
| | | 1 μM - No - <0% | |
| Trandolapril | Responder | 10 μM - Response - 67.75% | 1-10 |
| | | 1 μM - No - 0.63% | |
| Enalapril | Responder | 10 μM - Response - 67.90% | 1-10 |
| | | 1 μM - No - 1.55% | |
| Lisinopril | Non- | 10 μM -1.67% | 1-10 |
| | Responder | 1 μM - <0% | |
| Quinapril | Responder | 10 μM - Response - 62.29 | 1-10 |
| | | 1 μM - No - 4.87% | |
| | | 1 μM - No - 5.50% | |

### Example 8: Establishing an In Vitro Model System for Skin Penetration Screening

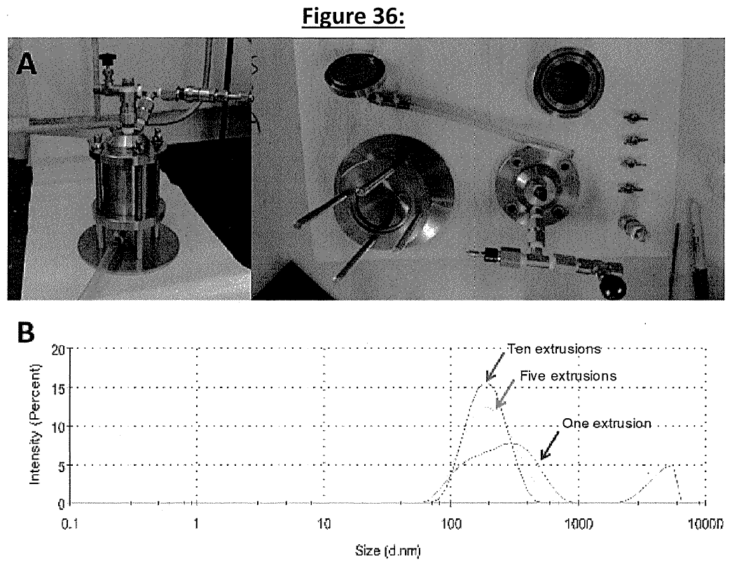

A series of experiments to demonstrate the capability for skin penetration and dermal absorption of beta blockers and ACE inhibitors were undertaken. Firstly, a custom-built Franz-diffusion cell (FIG. 34 A) was constructed in-house, consisting of a donor and receiver chamber, and fitted with a water-jacket to enable the temperature to be controlled to mimic physiological conditions. Initial screening investigations were performed using Strat-M® membranes—a synthetic membrane which mimics the permeation properties of human skin. This model system was used as a screening tool to increase the success of subsequent permeation studies performed on excised animal skin.

The system was validated by investigating the permeation of a water-soluble fluorescent dye, carboxyfluorescein, which has poor skin penetration properties. A solution of carboxyfluorescein (100 mM, 20 mM Na₂HPO₄, pH 7.4, 2 mL) was applied to the donor chamber, and the fluorescence of the receiver chamber monitored over a period of 120 hours. No permeation of the aqueous carboxyfluorescein through the skin-model membrane was observed over this time period (FIG. 34 B diamonds). As a comparison, carboxyfluorescein was encapsulated into deformable liposomes with a phospholipid membrane composition of soy phosphatidylcholine (Phospholipon® 90G) and polysorbate 80 in an 80:20 mol % ratio. Liposomes were prepared by dissolving the membrane components (20 mM total concentration; phospholipon 90G and polysorbate 80; 80:20 mol % ratio) in chloroform and removing the solvent in vacuo to form a thin lipid film. The film was rehydrated with carboxyfluorescein solution (100 mM carboxyfluorscein, 20 mM Na₂HPO₄, Ph 7.4) and extruded through 100 nm polycarbonate membranes to produce 100 nm deformable liposomes encapsulating carboxyfluorescein. The unencapsulated carboxyfluorescein was removed via dialysis against phosphate buffer (20 mM, pH 7.4). Even at low concentration (<1 mM), the deformable liposome formulation encapsulating carboxyfluorsein dramatically accelerated the permeation of carboxyfluorescein across the Strat-M® skin-model membranes over the same time period (FIG. 34 B squares).

Additionally, an emulsion-gel formulation containing 0.5% carboxyfluorescein was formulated and trialled in the permeation system. The formulation is described in Table 8. The carbomer was combined and dispersed with the water and carboxyfluorescein. The organic components were combined and thoroughly mixed. The organic phase is then combined with the aqueous phase and homogenised, to which the ammonia solution was subsequently added to initiate gelation.

The carboxyfluorescein emulgel (2 mL) was placed in the receiver chamber of the Franz-diffusion cell fitted with a Strat-M® membrane, and permeation of fluorescein across the membrane was monitored over time and quantified by fluoresce spectroscopy. The formulation displayed rapid permeation characteristics in the synthetic Strat-M® membrane system with a carboxyfluorescein flux of 4.6 μg cm⁻¹hr⁻¹ (FIG. 34 C). This is comparable with the flux of diclofenac across full-thickness human skin when using Voltaren® emulgel.

In summary, an in vitro assay system for determining the permeation of agents across human skin was established and validated. Additionally, emulsion gel formulations display enhanced permeation of active agents.

TABLE 8
Example of an emulsion-gel containing carboxyfluorescein.

| Ingredient | Amount | |
|---|---|---|
| Isopropyl alcohol | 1.25 | mL |
| Propylene glycol | 1.25 | mL |
| Coco-caprylate | 0.25 | mL |
| Liquid paraffin | 0.25 | mL |
| Polysorbate 80 | 0.2 | mL |
| Carbomer | 0.24 | g |
| Ammonia | 0.194 | mL |
| Carboxyfluorescein sodium salt | 2.7 | mL |
| | (100 mM carboxyfluorescein) | |
| Water | 4 | mL |

### Example 9: Emulsion Gel Formulations for Topical Application of Beta Blockers and ACE Inhibitors

A range of formulations for the topical application of β-blockers and ACE inhibitors to strawberry birthmarks was investigated (Table 9 below). The most successful in terms of ease-of-manufacture, preliminary stability and active pharmaceutical ingredient (API) compatibility is an emulsion-gel formulation. The formulations use a carbomer (Carbopol Ultrez 10 or 30) as the gel base, isopropyl alcohol and propylene glycol as skin permeation enhancers, liquid paraffin as an oil phase, and coco-caprylate as an emollient. Other gel bases were trialled, including carboxymethylcellulose, hydroxypropylcarboxymethylcellulose and chitosan; however, these tended to act only to increase viscosity as opposed to gelation.

A scalable route to the preparation of the gel has been established which utilizes a simple procedure (FIG. 35A). Firstly, the API is dissolved in water to produce an aqueous phase, to which the carbomer gel is subsequently dispersed. The organic components, namely paraffin liquid, isopropyl alcohol, propylene glycol, coco-caprylate, and polysorbate 80 are combined and mixed thoroughly to produce an organic phase. The organic phase is then slowly added to the aqueous phase and mixed thoroughly using a high-shear homogenizer to form a homogenous emulsion. The final stage involves adding a base, such as ammonia or sodium hydroxide, to neutralize the mixture and trigger gelation (FIG. 35 B). Light microscopy of the gel was performed after 3 months indicating a stable emulsion (FIG. 35C), and freeze-fracture transmission electron microscopy of the sample indicates emulsion droplets of approximately 0.5-2 μm in diameter (FIG. 35D).

TABLE 9
Examples of Topical formulations

| | | Propranolol | | Lisinopril and | | | | | |
|---|---|---|---|---|---|---|---|---|---|
| Lisinopril | | Hydrochloride | | Propranolol | | | | Lisinopril and | |
| Emulsion Gel | | Emulsion Gel | | Emulsion Gel | | Lisinopril Cream | | Propranolol Cream | |
| Lisinopril | 100 | Propranolol | 100 | Lisinopril | 100 | Lisinopril | 10 | Lisinopril | 100 |
| dihydrate | mg | Hydrochloride | mg | dihydrate | mg | dihydrate | mg | Dihydrate | mg |
| — | — | — | 500 | Propranolol | 100 | Transcutol | 30 | Propranolol | 100 |
| | | | mg | hydrochloride | mg | P | mg | Hydrochloride | mg |
| | | 2- | 1.25 | 2- | 500 | Cetyl | 40 | Transcutol P | 30 |
| | | hydroxypropyl- | mL | hydroxypropyl- | mg | Alcohol | mg | | mg |
| | | cyclodextrin | | cyclodextrin | | | | | |
| Isopropyl | 1.25 | Isopropyl | 1.25 | Isopropyl | 1.25 | Stearyl | 30 | Cetyl | 40 |
| alcohol | mL | alcohol | mL | alcohol | mL | Alcohol | mg | Alcohol | mg |
| Propylene | 1.25 | Propylene | 0.25 | Propylene | 1.25 | Polysorbate | 40 | Stearyl | 30 |
| glycol | mL | glycol | mL | glycol | mL | 60 | mg | Alcohol | mg |
| Coco- | 0.25 | Coco- | 0.25 | Coco- | 0.25 | Isopropyl | 22.5 | Polysorbate 60 | 40 |
| caprylate | | caprylate | mL | caprylate | mL | myristrate | mg | | mg |
| Liquid | 0.25 | Liquid | 0.2 | Liquid | 0.25 | Methyl | 0.5 | Isopropyl | 22.5 |
| Paraffin | | Paraffin | | Paraffin | | Paraben | | myristrate | mg |
| Polysorbate | 0.2 | Polysorbate 80 | 0.12 | Polysorbate 80 | 0.2 | Ethylene | 0.02 | Methyl | 0.5 |
| 80 | | | | | | diamine | | Paraben | |
| | | | | | | tetraacetic | | | |
| | | | | | | acid | | | |
| Carbomer | 0.12 | Carbomer | 0.97 | Carbomer | 0.12 | Purified | 818.5 | Ethylene | 0.02 |
| | | | | | | Water | | diamine | |
| | | | | | | | | tetraacetic acid | |
| Ammonia | 0.97 | Ammonia | 6.7 | Ammonia | 0.97 | Sodium | 0.5 | Purified | 818.5 |
| | | | | | | Hydroxide | | Water | |
| Water | 6.7 | Water | | Water | | Purified | 4.5 | Sodium | 0.5 |
| | | | | | | water for | | Hydroxide | |
| | | | | | | NaOH | | | |
| | | | | | | solution | | | |
| | | | | | | | | Purified | 4.5 |
| | | | | | | | | water for | |
| | | | | | | | | NaOH | |
| | | | | | | | | solution | |

The basic gel formulation was trialled with the ACE inhibitors enalapril and lisinopril, and β-blocker propranolol using the same formulation as previously described. Both ACE inhibitors could be easily formulated by dissolving the drug in the aqueous phase prior to the addition of the organic phase. However, the inclusion of 0.5-1 wt % propranolol caused precipitation as opposed to gelation at the final basification step. It was found that the inclusion of 2-hydroxypropyl-β-cyclodextrin at a 5:1 cyclodextrin:propranolol mass ratio inhibited this effect and allowed for stable gel formation. Furthermore, lisinopril and propranolol can be co-formulated at a concentration of 1 wt %, provided that 2-hydroxypropyl-β-cyclodextrin is included to prevent the precipitation of propranolol. These formulations are also amenable to a range of other beta blockers and ACE inhibitors, including timolol, nadolol, pindolol, sotalol, atenolol, betaxololol, ramipril, ramiprilat, quinapril, quinaprilat, captopril, trandalopril, trandaloprilat, enalapril, enalaprilat, cilazapril, cilazaprilat, benazepril and benazeprilat.

In summary, a range of topical cream and emulsion gel formulations were formulated, manufactured and trialled. This was an important demonstration that both beta blockers and ACE inhibitors can be formulated in stable compositions suitable for topical application, both alone as single agents, and as combinations of beta blockers and ACE inhibitors.

### Example 10: Liposome Gel Formulations for the Topical Application of Beta Blockers and ACE Inhibitors

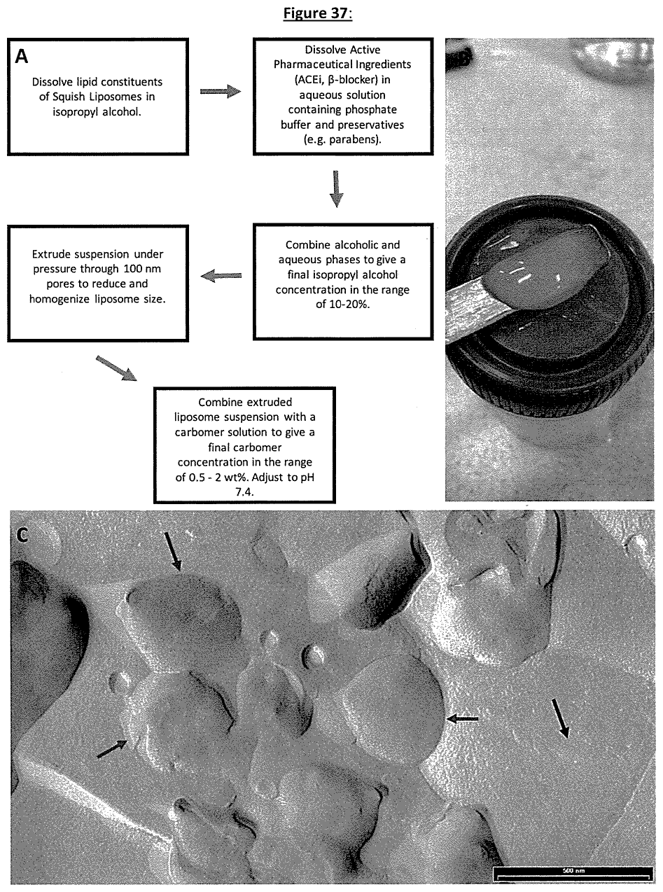

In addition to the formulation of an emulsion-gel for the application of ACE inhibitors and β-blockers, deformable liposomes and their incorporation in a topical gel base were also developed, along with a scalable route to manufacture. The lipid composition of 80:20 soy phosphatidylcholine and polysorbate 80 was used as this proved optimum in terms of both drug encapsulation and deformability. A custom-built, large-volume pressure extruder (FIG. 36A) was developed to increase the capacity of liposome production. This method reproducibly produced liposome batch sizes of up to 60 mL per run (c.f. 1 mL/run with manual extrusion), with good size control and polydispersity (FIGS. 36A and B).

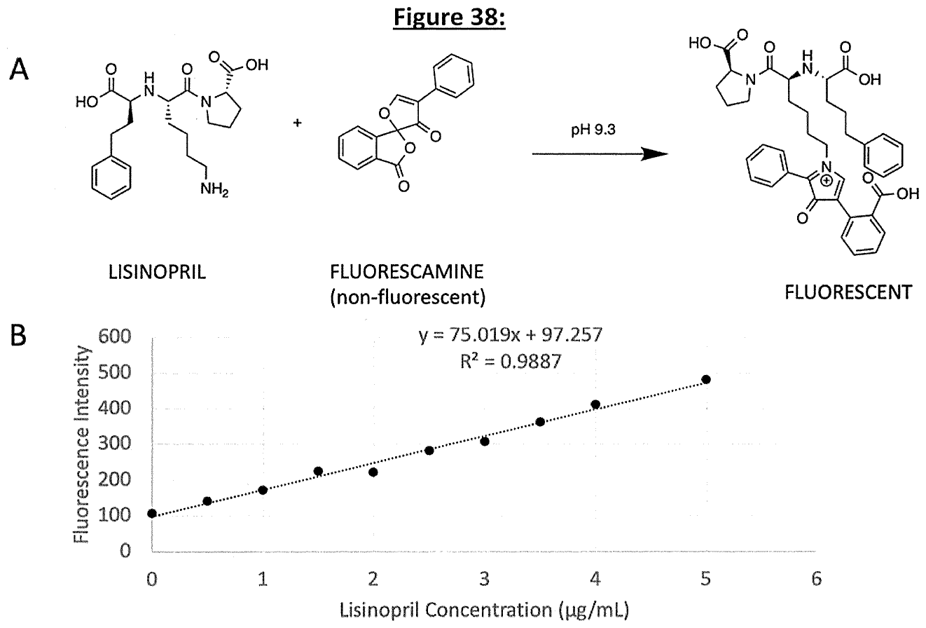

A five-step procedure was developed to manufacture the deformable liposomes in a gel form which can be applied topically to a strawberry birthmark (FIG. 37A). Firstly, the lipid constituents of the deformable liposomes, namely soy lecithin and polysorbate 80, were dissolved in isopropyl alcohol, while the APIs (ACE inhibitor, β-blocker, or combination thereof) were dissolved in an aqueous solution containing sodium phosphate buffered to pH 7.4. The organic phase was slowly added to the aqueous phase with stirring, and subsequently extruded through 100 nm membranes using the custom-built large volume pressure extruder (FIG. 36A). The extruded liposome suspension was combined with a suspension of carbomer to give a final carbomer concentration in the range of 0.2-1.5 wt %, mixed thoroughly to give a homogenous formulation, and subsequently gelled with the addition of a base such as sodium hydroxide, ammonia, or diethylammonia (FIG. 37B). Freeze fracture transmission electron microscopy was performed on the gel sample to investigate the morphology of the gel revealing liposomes of approximately 100-300 nm embedded within the gel matrix (FIG. 37 C).

In summary, a scalable route to the manufacture of deformable liposome gels suitable for topical application was developed. These formulations and methods are suitable for the inclusion of a wide variety of renin-angiotensin system inhibitors such as beta blockers and ACE inhibitors and ATIIR2 antagonists, either alone as single agents or in combination.

### Example 11: Detection of Beta Blockers and ACE Inhibitors Delivered Through Skin

Fluorescence Detection of ACE Inhibitor Lisinopril.

Attempts to quantify the permeation of lisinopril by fluorescence spectroscopy were made, as the limits of detection by mass spectrometry were too high to be useful for in vitro skin permeation studies. Lisinopril was chosen as a model ACE inhibitor due to the ease of chemical functionalisation, and therefore quantitative detection in vitro.

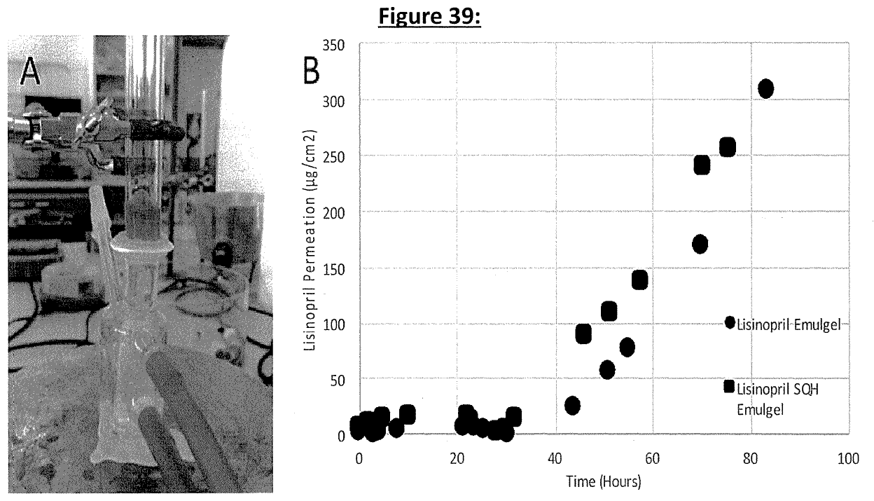

An assay was developed using fluorescamine, a non-fluorescent spirane compound which reacts readily under mild alkaline conditions with primary amines, generally in amino acids and peptides, to form stably and highly fluorescent conjugates (FIG. 38A). A reproducible linear relationship between fluorescence intensity and lisinopril concentration could be achieved (FIG. 38 B), with a detection limit of 0.1 μg mL⁻¹; sufficient for the quantification of lisinopril in the receiver chamber of Franz-diffusion experiments. Therefore, a protocol was developed whereby a sample of the receiver chamber solution is combined with alkaline buffer and an acetone solution of fluorescamine and compared to known quantities of lisinopril.

The procedure for analysis was developed as described. Firstly, the following solutions were prepared:
0.2 mg mL⁻¹ fluorescamine in acetone;
0.1 mg mL⁻¹ lisinopril solution in deionised water;
0.1 M boric acid buffer, pH 9.5;
pH 7.4 20 mm Na₂HPO₄ buffer (used in permeation experiments); and
a series of clean and dry 10 mL volumetric flasks.

A fluorescence standard curve (FIG. 9B) was prepared via the following procedure. To each 10-mL volumetric flask add:
1. 0.5 mL borate buffer;
2. 0, 10, 20, 40, 60, 80, 100 μl of a 0.05 mg mL⁻¹ lisinopril solution;
3. 0.6 mL—V (lisinopril solution added) of pH 7.4 phosphate buffer solution;
4. 1 mL fluorescamine solution; and
5. sufficient ethanol to make up to 10 mL.
6. Wait 10 minutes prior to measuring the fluorescence intensity via fluorescence spectroscopy using an excitation and emission wavelength of 390 nm and 487 nm respectively.

The procedure is amended for the quantification of lisinopril in the receiver chamber of a Franz-diffusion cell by replacing steps 2 and 3 with 0.6 mL of the receiver solution at the desired time points.

In summary, a fluorescence assay was developed for the sensitive and reproducible detection of ACE inhibitor lisinopril. This assay was useful in the quantification and confirmation of the permeation of ACE inhibitors through skin and skin-model membranes.

### Example 12: Delivery of Lisinopril Through Skin from Topical Liposome and Emulgel Formulations

The permeation characteristics of the lisinopril liposome gel and lisinopril emulgel formulations were assessed using the Franz diffusion assay system combined with the fluorescence assay developed for the quantification of lisinopril. Lisinopril was chosen as a model ACE inhibitor due to the ease of chemical functionalisation, and therefore quantitative detection in vitro.

The water-jacket of the Franz-diffusion cell was maintained at physiological temperature (37 degrees Celsius) for these experiments. Permeation experiments were performed using Strat-M® membranes—a synthetic membrane which mimics the permeation properties of human skin. The membrane is composed of two layers of polyethersulfone (PES) which is resistant to diffusion, on top of a more open and diffusive layer of polyolefin. These polymeric layers create a porous structure with a gradient of pore sizes and diffusivity across the membrane, which is further impregnated with a proprietary blend of synthetic lipids to create additional skin-like properties. The model system was used to minimize the complication of any primary amine containing contaminants arising in the receiver solution due to skin necrosis, and to enable comparison of the results to earlier permeation studies using emulsion gels containing 1% carboxyfluorescein. It was observed that the deformable liposome gel formulation is able to transport lisinopril across the model membrane at a rate of 5.6 μg cm⁻² hr⁻¹ (FIG. 39 B). A comparable permeation rate of 5.3 μg cm⁻² hr⁻¹ was observed using the 1% lisinopril emulgel (FIG. 39 B), which is similar in composition to Voltaren® (diclofenac) emulgel; a commercially available gel formulation known for rapid absorption in the case of its API diclofenac.

In comparison, our previous investigations using a 1% carboxyfluorescein emulgel of similar composition resulted in a permeation rate of 4.6 μg cm⁻² hr⁻¹. However, a long lag-time of approximately 30 and 37 hours was observed for lisinopril diffusion from the deformable liposome gel and emulgel respectively, whereas a lag-time of approximately 5 hours was observed for the permeation of carboxyfluorescein in the same in vitro system. As both lisinopril and carboxyfluorescein have comparable solubility's and molecular weights (>100 mg mL⁻¹ at pH 7.4, and 376 g mol⁻¹ and 405 g mol⁻¹ respectively), the prolonged lag-time is possibly due to the difference in lipophilicity (log P of 2.9 and −1.01 for carboxyfluorescein and lisinopril respectively). As lisinopril is a highly potent and long-acting ACE inhibitor, the inventors believe that the in vitro lag-time observed in this model system is not likely to be clinically significant. However, this demonstrates that other highly potent ACE inhibitors (such as ramipril, trandolapril, cilazapril and quinapril) with more favourable lipophilicities for transdermal transport (log P's of 2.9, 3.5, 0.8, and 3.2 respectively) may be preferred for formulation in topical cream and emulsion gel systems for the treatment of haemangiomas.

In summary, the results herein demonstrate that lisinopril, a model ACE inhibitor, can be effectively transported through human skin model membranes. Both emulsion gel and deformable liposome gel formulations produce similar diffusion rates, demonstrating that the rate of drug diffusion is primarily related to the concentration of drug in the formulation. Furthermore, the lag-time observed with lisinopril, a water-soluble ACE inhibitor (log P of −1.01) is greater than water soluble carboxyfluorescein (log P 2.9), which suggests other ACE inhibitors with more favourable lipophilicities may be more appropriate for rapid absorption during topical application. Overall, these results inform the design of topical compositions of ACE inhibitors suitable for the treatment of skin conditions, such as infantile haemangioma.

### Example 13: In Vitro Permeation of Propranolol Through Skin-Model Membranes

As the combination of an ACE inhibitor with a beta blocker was demonstrated to produce a significant improvement in the attenuation of haemangioma cell proliferation, an investigation was undertaken to ascertain the permeation rate of a beta blocker through skin. Propranolol was chosen as a model beta blocker as it can be accurately and rapidly quantified by UV-Vis spectroscopy.

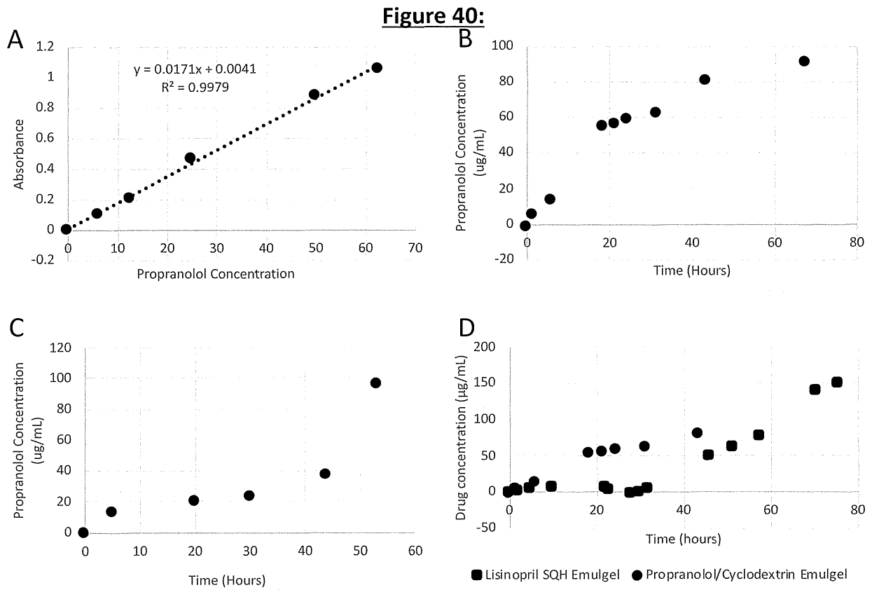

A standard curve for propranolol was constructed by measuring the absorbance at 288 nm of a propranolol solution (0.5 mg mL⁻¹, 20 mM Na₂HPO₄, pH 7.4) upon serial dilution to produce a concentration range of 0.5, 0.05, 0.025, 0.0125, 0.00625 mg mL⁻¹. The dilution series produced a linear correlation with absorbance, and a correlation coefficient (R²) of 0.99791 (FIG. 40A).

A 1% propranolol emulsion gel containing 2-hydroxypropyl-β-cyclodextrinl and a 1% propranolol cream were formulated as per Table 10 below for subsequent permeation studies.

Briefly, the 1% propranolol emulsion gel was manufactured by combining the appropriate proportions of organic components (isopropyl alcohol, propylene glycol, coco-caprylate, liquid paraffin, polysorbate 80). 2-hydroxypropyl-β-cyclodextrin was dissolved in the water component, following which propranolol hydrochloride was also dissolved. Carbomer was then dispersed in the aqueous propranolol solution, to which the organic phase was combined with vigorous mixing/homogenisation. Sodium hydroxide (1M solution) was subsequently added dropwise with stirring until a pH of 6-7 was obtained and the solution formed a gel.

The 1% propranolol cream was manufactured by the following procedure.
1. Combine cetyl alcohol, stearyl alcohol, polysorbate 60, and isopropyl myristate in a suitable container, heated to 65-70 degrees Celsius while mixing.
2. Propyl paraben was then added to the mixture and mixed to ensure complete dissolution while maintaining a temperature of 65-70 degrees Celsius.
3. Transcutol P was added to a separate containing and heated to 60-65 degrees Celsius, to which propranolol hydrochloride was added and mixed until dissolved, forming a slurry.
4. This was then combined with the molten organic mixture from Step 2 at 60-75 degrees Celsius.
5. Add purified water to a heated container with a mixer. Stir while heating to 65-70 degrees Celsius. Add EDTA and mix until dissolved. Add methylparaben and mix until dissolved. Maintain temperature at 65-70 degrees Celsius.
6. Combine the aqueous and organic phases and homogenise for 30 minutes with a high-shear homogenises, while maintaining the temperature at 65-70 degrees Celsius. Preferably this is done under vacuum.
7. Adjust pH to 5-5.6 with 1 M sodium hydroxide, measured at 25 degrees Celsius, followed by homogenisation for 5 minutes between each addition.
8. Transfer final solution to an appropriate container for storage. Mixture will thicken over time to form a semi-solid cream consistency.

The permeation of propranolol through skin-model membranes in vitro was assessed. A 2-mL volume of the appropriate vehicle (cream vs. emulsion gel) was placed in the donor chamber of the Franz-diffusion cell, and the concentration of propranolol in the receiver chamber was determined by UV-Vis spectroscopy at frequent time intervals over 60 hours. Permeation of propranolol from the emulsion gel vehicle occurred at a greater rate than from the cream vehicle (FIGS. 40 B and C), demonstrating that the emulsion gel vehicle promotes the absorption of the beta blocker to a greater and more rapid extent. A comparison of the 1% propranolol emulsion gel to an emulsion gel containing the ACE inhibitor lisinopril at a concentration of 1% was also made. A similar rate of drug flux is evident from the propranolol and lisinopril samples. However, the penetration of the beta blocker occurs far more quickly than that of the ACE inhibitor, displaying little lag-time (FIG. 40 D).

In summary, the permeation of beta blocker propranolol across skin membranes was performed demonstrating that propranolol can be rapidly absorbed through skin when formulated in emulsion gel and cream formulations, suitable for topical application. Furthermore, these results demonstrate that beta blockers surprisingly absorb more rapidly than ACE inhibitors. Therefore, topical fixed-dose combinations of ACE inhibitors and beta blockers will require a greater proportion of ACE inhibitor relative to beta blocker in their manufacture in order to obtain similar rates of absorption. These results provide critical insight into the formulation of fixed-dose combination products containing ACE inhibitors and beta blockers for the treatment of haemangiomas.

TABLE 10
Example formulation of a cream and an emulsion-gel
containing propranolol for topical application.

| 1% Propranolol Emusion Gel | | | 1% Propranolol Cream | | |
|---|---|---|---|---|---|
| Ingredient | Amount | | Ingredient | Amount | |
| Isopropyl alcohol | 1.25 | mL | Cetyl alcohol | 200 | mg |
| Propylene glycol | 1.25 | mL | Stearyl alcohol | 150 | mg |
| Coco-caprylate | 0.25 | mL | Polysorbate 60 | 200 | mg |
| Liquid paraffin | 0.25 | mL | Isopropyl myristate | 112.5 | mg |
| Polysorbate 80 | 0.2 | mL | Propylparaben | 2.5 | mg |
| Carbomer | 0.12 | g | Propranolol hydrochloride | 55 | mg |
| Water | 6.7 | mL | Transcutol P | 150 | mg |
| Propranolol hydrochloride | 104 | mg | Methylparaben | 2.5 | mg |
| 2-Hydroxypropyl-β- | 600 | mg | Water | 4092.5 | mg |

| | | | | |
|---|---|---|---|---|
| cyclodextrin | | | | |
| Sodium hydroxide | 1 M added until | Ethylene diamine tetraacetic | 0.02 | mg |

| | a pH of 6-7. | | acid (EDTA) | |
|---|---|---|---|---|
| Propylparaben | 2.5 | mg | Sodium hydroxide | 1 M solution |
| | | | | added until pH |
| | | | | 5-6. |
| Methylparaben | 2.5 | mg | | |
| Ethylenediamine tetraacetic | 0.02 | MG | | |

acid (EDTA)

### Example 14: Fixed-Dose Topical Combination Cream

This example relates to the formulation of a topical fixed-dose combination product containing an ACE inhibitor and a beta blocker for the treatment of haemangiomas. The formulations described serve as examples only and are not intended to limit the scope of the invention in any way. The formulations were found to possess good stability.

The exemplar fixed-dose topical formulations can be manufactured via the following procedure.
1. Combine cetyl alcohol, stearyl alcohol, polysorbate 60, and isopropyl myristate in a suitable container, heated to 65-70 degrees Celsius while mixing.
2. Add Propyl paraben to the mixture and mixe to ensure complete dissolution while maintaining a temperature of 65-70 degrees Celsius.
3. Add Transcutol P to a separate container and heat to 60-65 degrees Celsius. Add the appropriate amount of beta blocker and/or ACE inhibitor and mix until dissolved, forming a slurry.
4. Combine the slurry with the molten organic mixture from Step 2 at 60-75 degrees Celsius. 5. Add purified water to a heated container with a mixer. Stir while heating to 65-70 degrees Celsius. Add EDTA and mix until dissolved. Add methylparaben and mix until dissolved. Maintain temperature at 65-70 degrees Celsius.
6. Combine the aqueous and organic phases and homogenise for 30 minutes with a high-shear homogenises, while maintaining the temperature at 65-70 degrees Celsius. Preferably this is done under vacuum.
7. Adjust pH to 5-5.6, or 6-7 with 1 M sodium hydroxide, measured at 25 degrees Celsius, followed by homogenisation for 5 minutes between each addition.
8. Transfer final solution to an appropriate container for storage. Mixture will thicken over time to form a semi-solid cream consistency.

TABLE 11
Example of fixed-dose beta blocker and
ACE inhibitor formulations for topical application.

| 1% Propranolol and 1% Ramipril Cream | | | 2% Trandolapril and 0.5% Timolol Cream | | |
|---|---|---|---|---|---|
| Ingredient | Amount | | Ingredient | Amount | |
| Ramipril | 50 | mg | Trandalopril | 100 | mg |
| Propranolol hydrochloride | 55 | mg | Timolol maleate | 34 | mg |
| Cetyl alcohol | 200 | mg | Cetyl alcohol | 200 | mg |
| Stearyl alcohol | 150 | mg | Stearyl alcohol | 150 | mg |
| Polysorbate 60 | 200 | mg | Polysorbate 60 | 200 | mg |
| Isopropyl myristate | 112.5 | mg | Isopropyl myristate | 112.5 | mg |
| Propylparaben | 2.5 | mg | Propylparaben | 2.5 | mg |
| Transcutol P | 150 | mg | Transcutol P | 150 | mg |
| Methylparaben | 2.5 | mg | Methylparaben | 2.5 | mg |
| Water | 4077.5 | mg | Water | 4048.5 | mg |
| Ethylene diamine tetraacetic | 0.02 | mg | Ethylene diamine | 0.02 | mg |

| | | | |
|---|---|---|---|
| acid (EDTA) | | tetraacetic acid (EDTA) | |
| Sodium hydroxide | 1M solution | Sodium hydroxide | 1M solution |
| | added until pH | | added until pH |
| | 5-6. | | 6-7. |

The entire disclosures of all applications, patents and publications, cited above and below, if any, are hereby incorporated by reference. However, the reference to any applications, patents, and publications in this specification is not, and should not be taken as, an acknowledgement or any form of suggestion that they constitute valid prior art or form part of the common general knowledge in any country in the world.

Throughout this specification and any claims which follow, unless the context requires otherwise, the word “comprise”, “comprising” and the like, are to be construed in an inclusive sense as opposed to an exclusive sense, that is to say, in the sense of “including, but not limited to”.

Titles, heading, or the like are provided herein to enhance the reader's comprehension of the document and should not be read as limiting the scope of the invention.

1-32. (canceled)

33. A composition comprising: an ACEi and a beta-blocker; an ACEi and an ATIIR2 antagonist; a beta-blocker and an ATTIIR2 antagonist; or, an ACEi, a beta-blocker, and an ATIIR2 antagonist; wherein the composition is suitable for local administration.

34. (canceled)

35. A composition as claimed in claim 33, wherein the composition comprises two or more ACEi, two or more beta-blockers and/or two or more ATIIR2 antagonists.

36. A composition as claimed in claim 33, wherein the composition comprises a lower amount of beta-blocker relative to the amount of ACEi.

37. A composition as claimed in claim 33, wherein the ACEi is a prodrug.

38. A composition as claimed in claim 33, wherein the ACEi is chosen from the group consisting of: Cilazapril, Enalapril, Ramipril, trandolapril, Quinapril, Benazepril, and Captopril.

39. A composition as claimed in claim 33, wherein the beta-blocker is substantially in the R-enantiomer form, or comprises a racemic mixture having at least a predominance of the R-enantiomer form.

40. A composition as claimed in claim 33, wherein the beta-blocker is a non-selective beta-blocker.

41. A composition as claimed in claim 33, wherein the beta-blocker is chosen from the group consisting of: R-Propranolol; R/S Propranolol; R-Timolol; R/S Timolol; and, S-Timolol.

42. A composition as claimed in claim 33, wherein the ATIIR2 antagonist is chosen from the group consisting of: L-159,686; and, EMA401.

43. A composition as claimed in claim 33, wherein the composition comprises: Cilazapril and R-propranolol; a) Ramipril or enalapril or trandolapril and b) R-propranolol; a) Cilazapril and b) S-propranolol or racemic (R/S) propranolol; a) Ramipril or enalapril or trandolapril and b) S-propranolol or racemic (R/S) propranolol; Cilazapril and timolol; a) Ramipril or enalapril or trandolapril and b) timolol; a) L-159,686 or EMA401 and b) R-propranolol; a) L-159,686 or EMA401 and b) S-propranolol or racemic propranolol; a) L-159,686 or EMA401 and b) timolol; a) L-159,686 or EMA401 and b) cilazapril; or, a) L-159,686 or EMA401 and b) Ramipril or enalapril or trandolapril.

44. A composition as claimed in claim 33, wherein the composition comprises: R-timolol and cilazapril; S-timolol and cilazapril; R/S-timolol and cilazapril; R-propranolol and cilazapril; S-propranolol and cilazapril; R/S-propranolol and cilazapril; L-159,686 and cilazapril; R-propranolol and L-159,686; S-propranolol and L-159,686; R/S-propranolol and L-159,686; L-159,686 and S-timolol; L-159,686 and R-timolol; EMA401 and S-timolol; EMA401 and R-timolol; EMA401 and cilazapril; EMA401 and R-propranolol; EMA401 and S-propranolol; or, EMA401 and R/S-propranolol.

45. (canceled)

46. A composition as claimed in claim 33, wherein the composition comprises: a) an ACEi in prodrug form and a non-selective beta blocker; b) a non-selective beta-blocker and an ATIIR2 antagonist; or, an ATIIR2 antagonist and an ACEi in prodrug form.

47. A method of treating hemangioma in a human patient by administering to the patient an ACEi and a beta-blocker.

48. A composition according to claim 33, which is suitable for treating hemangioma.

49. A composition as claimed in claim 48, wherein the composition is suitable for topical administration.
